(19) United States
(12) Patent Application Publication
(10) Pub. No.: US 2026/0028423 A1
(43) Pub. Date: Jan. 29, 2026

(54) PREDICTION OF AN INCREASE OF DPP3 IN A PATIENT WITH SEPTIC SHOCK

(71) Applicant: 4TEEN4 Pharmaceuticals GmbH, Hennigsdorf (DE)

(72) Inventor: Andreas Bergmann, Berlin (DE)

(73) Assignee: 4TEEN4 Pharmaceuticals GmbH, Hennigsdorf (DE)

(21) Appl. No.: 18/998,052

(22) PCT Filed: Jul. 31, 2023
(86) PCT No.: PCT/EP2023/071214

§ 371 Date: Jan. 23, 2025

(30) Foreign Application Priority Data
Jul. 29, 2022 (EP) 22187933.1
Jul. 29, 2022 (EP) 22187940.6

(51) Int. Cl. C07K 16/40; A61K 39/00; A61P 37/06; G01N 33/573

(52) U.S. Cl. CPC C07K 16/40; A61P 37/06; C12Y 304/14004; G01N 33/573; A61K 2039/505; C07K 2317/24; C07K 2317/92; G01N 2333/948

(57) ABSTRACT

The invention relates to a method for the prediction of an increase of dipeptidyl peptidase 3 (DPP3) in a critically ill patient. In particular, the method comprises providing a sample from said patient, determining a level of Dipeptidyl peptidase 3 (DPP3) in said sample, comparing said level to a pre-determined threshold, wherein the level of DPP3 in said sample is indicative of an increase of DPP3 if said level of DPP3 is above a pre-determined threshold level, which is in the range between 22 and 40 ng/ml. Furthermore, the invention also relates to a method for the prevention of a DPP3 increase in a critical ill patient, wherein a DPP3 inhibitor is administered to the patient if the level of DPP3 is above a threshold between 40 ng/ml and 22 ng/ml and said DPP3 inhibitor is an anti-DPP3-antibody and/or and anti-DPP3-antibody fragment and/or anti-DPP3 scaffold. Moreover, the invention also relates to a DPP3 inhibitor for use in the prevention of a DPP3 increase in a critical ill patient.

### FIELD OF THE INVENTION

The invention relates to a method for the prediction of an increase of dipeptidyl peptidase 3 (DPP3) in a critically ill patient. Furthermore, the invention also relates to a method for the prevention of a DPP3 increase in a critical ill patient, wherein a DPP3 inhibitor is administered to the patient if the level of DPP3 is above a threshold between 40 ng/ml and 22 ng/ml and said DPP3 inhibitor is an anti-DPP3-antibody and/or and anti-DPP3-antibody fragment and/or anti-DPP3 scaffold. Moreover, the invention also relates to a DPP3 inhibitor for use in the prevention of a DPP3 increase in a critical ill patient.

### BACKGROUND

Dipeptidyl peptidase 3—also known as Dipeptidyl aminopeptidase III, Dipeptidyl arylamidase III, Dipeptidyl peptidase III, Enkephalinase B or red cell angiotensinase; short name: DPP3, DPPIII—is a metallopeptidase that removes dipeptides from physiologically active peptides, such as enkephalins and angiotensins. DPP3 was first identified, and its activity measured in extracts of purified bovine anterior pituitary by Ellis & Nuenke 1967. The enzyme, which is listed as EC 3.4.14.4, has a molecular mass of about 83 kDa and is highly conserved in procaryotes and eucaryotes (Prajapati & Chauhan 2011). The amino acid sequence of the human variant is depicted in SEQ ID NO 36. Dipeptidyl peptidase III is a mainly cytosolic peptidase which is ubiquitously expressed. Despite lacking a signal sequence, a few studies reported membranous activity (Lee & Snyder 1982).

DPP3 is a zinc-depending exo-peptidase belonging to the peptidase family M49. It has a broad substrate specificity for oligopeptides from three or four to ten amino acids of various compositions and is also capable of cleaving after proline. DPP3 is known to hydrolyze dipeptides from the N-terminus of its substrates, including angiotensin II, III and IV; Leu- and Met-enkephalin; endomorphin 1 and 2. The metallopeptidase DPP3 has its activity optimum at pH 8.0-9.0 and can be activated by addition of divalent metal ions, such as Co²⁺ and Mg²⁺.

Structural analysis of DPP3 revealed the catalytic motifs HELLGH (hDPP3 450-455) and EECRAE (hDPP3 507-512), as well as following amino acids, that are important for substrate binding and hydrolysis: Glu316, Tyr, 318, Asp366, Asn391, Asn394, His568, Arg572, Arg577, Lys666 and Arg669 (Prajapati & Chauhan 2011; Kumar et al. 2016; numbering refers to the sequence of human DPP3, see SEQ ID NO. 36). Considering all known amino acids or sequence regions that are involved in substrate binding and hydrolysis, the active site of human DPP3 can be defined as the area between amino acids 316 and 669.

The most prominent substrate of DPP3 is angiotensin II (Ang II), the main effector of the renin-angiotensin system (RAS). The RAS is activated in cardiovascular diseases (Dostal et al. 1997. J Mol Cell Cardiol 29:2893-902; Roks et al. 1997. Heart Vessels. Suppl 12:119-24), sepsis, and septic shock (Correa et al. 2015. Crit Care 19:98). In particular, Ang II has been shown to modulate many cardiovascular functions including the control of blood pressure and cardiac remodeling.

Recently, two assays were generated, characterized, and validated to specifically detect DPP3 in human bodily fluids (e.g., blood, plasma, serum): a luminescence immunoassay (LIA) to detect DPP3 protein concentration and an enzyme capture activity assay (ECA) to detect specific DPP3 activity (Rehfeld et al. 2019. JALM 3(6): 943-953). A washing step removes all interfering substances before the actual detection of DPP3 activity is performed. Both methods are highly specific and allow the reproducible detection of DPP3 in blood samples.

Circulating DPP3 levels were shown to be increased in cardiogenic shock patients and were associated with an increased risk of short-term mortality and severe organ dysfunction (Deaniau et al. 2020. Eur J Heart Fail. 22(2):290-299). Moreover, DPP3 measured at inclusion discriminated cardiogenic shock patients who did develop refractory shock vs. non-refractory shock and a DPP3 concentration ≥59.1 ng/mL was associated with a greater risk of death (Takagi et al. 2020. Eur J Heart Fail. 22(2):279-286).

The peptide adrenomedullin (ADM) was described for the first time in 1993 (Kitamura et al., 1993. Biochem Biophys Res Comm 192 (2): 553-560) as a novel hypotensive peptide comprising 52 amino acids, which had been isolated from a human pheochromocytoma cell line (SEQ ID No.: 20). In the same year, cDNA coding for a precursor peptide comprising 185 amino acids and the complete amino acid sequence of this precursor peptide were also described. The precursor peptide, which comprises, inter alia, a signal sequence of 21 amino acids at the N-terminus, is referred to as “pre-proadrenomedullin” (pre-proADM). In the present description, all amino acid positions specified usually relate to the pre-proADM, which comprises the 185 amino acids. The peptide ADM is a peptide which comprises 52 amino acids (SEQ ID No: 20) and which comprises the amino acids 95 to 146 of pre-proADM, from which it is formed by proteolytic cleavage. To date, substantially only a few fragments of the peptide fragments formed in the cleavage of the pre-proADM have been more exactly investigated, in particular the physiologically active peptides ADM and “PAMP”, a peptide comprising 20 amino acids (22-41), which follows the 21 amino acids of the signal peptide in pre-proADM. The discovery and characterization of ADM in 1993 triggered intensive research activity, the results of which have been summarized in various review articles, in the context of the present description, reference being made in particular to the articles to be found in an issue of “Peptides” devoted to ADM in particular (Takahashi 2001. Peptides 22: 1691; Eto 2001. Peptides 22: 1693-1711). A further review is Hinson et al. 2000 (Hinson et al. 2000. Endocrine Reviews 21(2):138-167). In the scientific investigations to date, it has been found, inter alia, that ADM may be regarded as a polyfunctional regulatory peptide. It is released into the circulation in an inactive form extended by glycine (Kitamura et al. 1998. Biochem Biophys Res Commun 244(2): 551-555). There is also a binding protein (Pio et al. 2001. The Journal of Biological Chemistry 276(15): 12292-12300), which is specific for ADM and probably likewise modulates the effect of ADM. Those physiological effects of ADM as well as of PAMP, which are of primary importance in the investigations to date, were the effects influencing blood pressure.

Hence, ADM is an effective vasodilator, and thus it is possible to associate the hypotensive effect with the particular peptide segments in the C-terminal part of ADM. It has furthermore been found that the above-mentioned physiologically active peptide PAMP formed from pre-proADM likewise exhibits a hypotensive effect, even if it appears to have an action mechanism differing from that of ADM (in addition to the above-mentioned review articles Eto et al. 2001 and Hinson et al. 2000 see also Kuwasaki et al. 1997. FEBS Lett 414(1): 105-110; Kuwasaki et al. 1999. Ann. Clin. Biochem. 36: 622-628; Tsuruda et al. 2001 Life Sci. 69(2): 239-245 and EP-A2 0 622 458). It has furthermore been found, that the concentrations of ADM, which can be measured in the circulation and other biological liquids, are in a number of pathological states, significantly above the concentrations found in healthy control subjects. Thus, the ADM level in patients with congestive heart failure, myocardial infarction, kidney diseases, hypertensive disorders, diabetes mellitus, in the acute phase of shock and in sepsis and septic shock are significantly increased, although to different extents. The PAMP concentrations are also increased in some of said pathological states, but the plasma levels are lower relative to ADM (Eto 2001. Peptides 22: 1693-1711). It was reported that unusually high concentrations of ADM are observed in sepsis, and the highest concentrations in septic shock (Eto 2001. Peptides 22: 1693-1711; Hirata et al. Journal of Clinical Endocrinology and Metabolism 81(4): 1449-1453; Ehlenz et al. 1997. Exp Clin Endocrinol Diabetes 105: 156-162; Tomoda et al. 2001. Peptides 22: 1783-1794; Ueda et al. 1999. Am. J. Respir. Crit. Care Med. 160: 132-136 and Wang et al. 2001. Peptides 22: 1835-1840).

Plasma concentrations of ADM are elevated in patients with heart failure and correlate with disease severity (Hiravama et al. 1999. J Endocrinol 160: 297-303; Yu et al. 2001. Heart 86: 155-160). High plasma ADM is an independent negative prognostic indicator in these subjects (Poyner et al. 2002. Pharmacol Rev 54: 233-246).

WO2004/097423 describes the use of an antibody against ADM for diagnosis, prognosis, and treatment of cardiovascular disorders. Treatment of diseases by blocking the ADM receptor are also described in the art, (e.g., WO2006/027147, PCT/EP2005/012844) said diseases may be sepsis, septic shock, cardiovascular diseases, infections, dermatological diseases, endocrinological diseases, metabolic diseases, gastroenterological diseases, cancer, inflammation, hematological diseases, respiratory diseases, muscle skeleton diseases, neurological diseases, urological diseases.

It is reported for the early phase of sepsis that ADM improves heart function and the blood supply in liver, spleen, kidney, and small intestine. Anti-ADM-neutralizing antibodies neutralize the before mentioned effects during the early phase of sepsis (Wang et al. 2001. Peptides 22: 1835-1840).

For other diseases blocking of ADM may be beneficial to a certain extent. However, it might also be detrimental if ADM is totally neutralized, as a certain amount of ADM may be required for several physiological functions. In many reports it was emphasized, that the administration of ADM may be beneficial in certain diseases. In contrast thereto, in other reports ADM was reported as being life threatening when administered in certain conditions.

WO2013/072510 describes a non-neutralizing anti-ADM antibody for use in therapy of a severe chronical or acute disease or acute condition of a patient for the reduction of the mortality risk for said patient.

WO2013/072511 describes a non-neutralizing anti-ADM antibody for use in therapy of a chronical or acute disease or acute condition of a patient for prevention or reduction of organ dysfunction or organ failure.

WO2013/072512 describes a non-neutralizing anti-ADM antibody that is an ADM stabilizing antibody that enhances the half-life (t_{1/2} half retention time) of ADM in serum, blood, plasma. This ADM stabilizing antibody blocks the bioactivity of ADM to less than 80%.

WO2013/072513 describes a non-neutralizing anti-ADM antibody for use in therapy of an acute disease or condition of a patient for stabilizing the circulation.

WO2013/072514 describes a non-neutralizing anti-ADM antibody for regulating the fluid balance in a patient having a chronic or acute disease or acute condition.

WO2017/182561 describes methods for determining the total amount or active DPP3 in a sample of a patient for the diagnosis of a disease related to necrotic processes. It further describes a method of treatment of necrosis-related diseases by antibodies directed to DPP3.

WO2021/170838 describes a method for therapy guidance and/or therapy monitoring and/or therapy stratification in patients with shock and patients running into shock by determining the level of DPP3 and if the level is below a threshold of preferably 50 ng/ml, then administering an anti-ADM antibody. High DPP3 blood levels are associated with higher organ dysfunction scores, the need of cardiovascular support and the development of myocardial dysfunction, refractory shock, acute kidney injury and increased short-term mortality. Based on these clinical associations, it is plausible that active DPP3 released in the blood of shock patients is a factor contributing to the deterioration of vascular tone in shock syndromes by disabling the vasopressor effects of endogenous Ang II (Malovan et al. 2023 FEBS Journal 290(9):2246-2262). In patients with shock the presence of endothelial dysfunction is detected with the help of the biomarker bio-ADM, whereas cardiac dysfunction is detected by measuring DPP3. DPP3 above a threshold has been used as exclusion criteria for the application of medicaments addressing a different pathway than DPP3, e.g., anti-ADM antibodies, in particular the anti-ADM antibody Adrecizumab, which is directed against the N-terminus of ADM. Adrecizumab therapy showed a better efficacy if patients with DPP3 above 50 ng/ml are excluded from treatment (WO2021/170838). However, in patients with an initial DPP3 level below 50 ng/ml, the DPP3 level may increase in the following time above 50 ng/ml, which would indicate an exclusion for the application of medicaments addressing a different pathway than DPP3. Hence, it was the surprising finding of the present invention that if DPP3 is well below the threshold of 50 ng/ml, preferably below a threshold of ≤40 ng/ml or in the range between 22 ng/ml and 40 ng/ml in a critically ill patient, said patient will not likely experience an increase in DPP3 during follow-up. On the other hand, this means, that if DPP3 is above a threshold of 40 ng/ml, preferably above a threshold in the range between 40 ng/ml and 22 ng/ml in a critically ill patient, said patient will more likely experience an increase in DPP3 during follow-up. Moreover, the inventors found that the increase is a short-term increase that will occur within hours or up to 7 days, respectively.

As a consequence, this would mean that patients with a level of DPP3 below a threshold in the range between 22 ng/ml and 40 ng/ml may be treated with a medicament addressing a different pathway than DPP3, e.g., anti-ADM antibodies, in particular the anti-ADM antibody Adrecizumab, and that patients with a level of DPP3 above a threshold in the range between 40 ng/ml and 22 ng/ml may not be treated with a medicament addressing a different pathway than DPP3, e.g., anti-ADM antibodies, in particular the anti-ADM antibody Adrecizumab.

It is the aim of the present invention to reduce the number of critically ill patients that will experience a short-term increase (e.g., within hours up to 7 days) of the level of DPP3 above a critical cut-off (e.g., above 40 ng/ml or 50 ng/ml), which means to pursue a high sensitivity of the test for this endpoint. It is also the surprising finding of the present invention to predict the non-increase of DPP3 above a critical cut-off in critically ill patients during follow-up, wherein the level of DPP3 is below a pre-determined threshold level, which is in the range between 22 and 40 ng/ml. In this way, patients can be identified that could suitably be treated with a medicament addressing a different pathway than DPP3, e.g., anti-ADM antibodies, in particular the anti-ADM antibody Adrecizumab. Surprisingly, a “point of no return”, where DPP3 levels will further increase above a critical cut-off in a critically ill patients during short-term follow-up, is reached already at a much lower DPP3 level than known in the art. Identifying patients, in particular identifying patients as early as possible, that will not experience such short-term increase of the level of DPP3 above a critical cut-off significantly improves the overall efficacy of treatment for the patient population, which might be standard of care treatment, or medications addressing a different pathway than DPP3, e.g., anti-ADM antibodies, in particular the anti-ADM antibody Adrecizumab.

### DESCRIPTION OF THE INVENTION

Subject matter of the present invention is a method for the prediction of an increase of dipeptidyl peptidase 3 (DPP3) in a critically ill patient, the method comprising:
determining the level of DPP3 in a sample of bodily fluid of said patient,
comparing said level of determined DPP3 to a pre-determined threshold,
wherein said predetermined threshold of DPP3 is in the range between 40 ng/ml and 22 ng/ml,
wherein a level of DPP3 in said sample above said pre-determined is indicative for an increase of DPP3 in said patient.

One embodiment of the present application relates to a method for the prediction of an increase of DPP3 in a critically ill patient, wherein said patient is a patient with severe infection, sepsis, heart failure, chronic heart failure, acute heart failure (AHF), myocardial infarction (MI), stroke, a patient with organ dysfunction or organ failure (e.g. dysfunction or failure of liver, kidney, lung), a patient undergoing major surgery, a patient with trauma (e.g. burn trauma, polytrauma), a patient with shock and/or a patient running into shock.

One embodiment of the present application relates to a method for the prediction of an increase of DPP3 in a critically ill patient, wherein said patient has a shock or is running into shock, wherein said shock is selected from the group comprising shock due to hypovolemia, cardiogenic shock, obstructive shock and distributive shock, in particular cardiogenic shock or septic shock.

One preferred embodiment of the present application relates to a method for the prediction of an increase of DPP3 in a critically ill patient, wherein said patient has a shock and/or is running into shock, wherein
in case of cardiogenic shock said patient may have suffered an acute coronary syndrome (e.g., acute myocardial infarction) or wherein said patient has heart failure (e.g., acute decompensated heart failure), myocarditis, arrhythmia, cardiomyopathy, valvular heart disease, aortic dissection with acute aortic stenosis, traumatic chordal rupture or massive pulmonary embolism, or
in case of hypovolemic shock said patient may have suffered a hemorrhagic disease including gastrointestinal bleed, trauma, vascular etiologies (e.g., ruptured abdominal aortic aneurysm, tumor eroding into a major blood vessel) and spontaneous bleeding in the setting of anticoagulant use or a non-hemorrhagic disease including vomiting, diarrhea, renal loss, skin losses/insensible losses (e.g., burns, heat stroke) or third-space loss in the setting of pancreatitis, cirrhosis, intestinal obstruction, trauma, or
in case of obstructive shock said patient may have suffered a cardiac tamponade, tension pneumothorax, pulmonary embolism or aortic stenosis, or
in case of distributive shock said patient may have septic shock, neurogenic shock, anaphylactic shock or shock due to adrenal crisis.

Another embodiment of the present application relates to a method for the prediction of an increase of DPP3 in acratically ill patient, wherein said pre-determined threshold of DPP3 in a sample of bodily fluid of said subject is in the range between 40 ng/ml and 22 ng/mL.

Another embodiment of the present application relates to a method for the prediction of an increase of DPP3 in a critically ill patient, wherein said pre-determined threshold of DPP3 in a sample of bodily fluid of said subject is in the range between 30 ng/ml and 22 ng/ml.

Another embodiment of the present application relates to a method for the prediction of an increase of DPP3 in a critically ill patient, wherein said pre-determined threshold of DPP3 in a sample of bodily fluid of said subject is in the range between 25 ng/ml and 22 ng/ml.

Another embodiment of the present application relates to a method for the prediction of an increase of DPP3 in a critically ill patient, wherein said predicted increase is an increase to DPP3 levels equal to or above 40 ng/ml, preferred equal to or above 50 ng/ml.

Another embodiment of the present application relates to a method for the prediction of an increase of DPP3 in a critically ill patient, wherein said predicted increase of the DPP3 level is equal to or above 10%, more preferred equal to or above 20%, even more preferred equal to or above 40%, even more preferred equal to or above 50% even more preferred equal to or above 75%, even more preferred equal to or above 100%. In particular, this relates to the increase of the DPP3 level over the level of DPP3 determined in the sample.

Another embodiment of the present application relates to a method for the prediction of an increase of DPP3 in a critically ill patient, wherein said predicted increase of the DPP3 level is equal to or above 2.5 ng/ml, more preferred equal to or above 5 ng/ml, even more preferred equal to or above 10 ng/ml, even more preferred equal to or above 15 ng/ml, even more preferred equal to or above 20 ng/ml, even more preferred equal to or above 25 ng/ml. In particular, this relates to the increase of the DPP3 level over the level of DPP3 determined in the sample.

Another embodiment of the present application relates to a method for the prediction of an increase of DPP3 in a critically ill patient, wherein a patient is selected for therapy/treatment if the level of a DPP3 in said sample is below said pre-determined threshold, wherein said therapy is selected from the group of alkaline phosphatase, immune suppressors, corticosteroids, vasopressors, fluids, anti-ADM antibodies or antibody fragments or scaffolds.

Another embodiment of the present application relates to a method for the prediction of an increase of DPP3 in a critical ill patient, wherein a patient is selected for therapy/treatment with DPP3 inhibitors if the level of a DPP3 in said sample is above said pre-determined threshold, wherein said DPP3 inhibitor is selected from the group of anti-DPP3-antibodies or anti-DPP3-antibody fragments or anti-DPP3 scaffolds.

Another specific embodiment of the present application relates to a method for the prediction of an increase of DPP3 in a critically ill patient, wherein either the level of DPP3 protein and/or the level of active DPP3 is determined and compared to a pre-determined threshold.

Another preferred embodiment of the present application relates to a method for the prediction of an increase of DPP3 in a critically ill patient, wherein the level of DPP3 is determined by contacting said sample of bodily fluid with a capture binder that binds specifically to DPP3.

One embodiment of the present application relates to a method for the prediction of an increase of DPP3 in a critically ill patient, wherein said determination comprises the use of a capture-binder that binds specifically to full-length DPP3, wherein said capture-binder may be selected from the group of antibody, antibody fragment or non-IgG scaffold.

A further embodiment of the present application relates to a method for the prediction of an increase of DPP3 in a critically ill patient, wherein the amount of DPP3 protein and/or DPP3 activity is determined in a bodily fluid sample of said subject and wherein said determination comprises the use of a capture-binder that binds specifically to full-length DPP3, wherein said capture-binder is an antibody.

One embodiment of the present application relates to a method for the prediction of an increase of DPP3 in a critically ill patient, wherein the amount of DPP3 protein and/or DPP3 activity is determined in a bodily fluid sample of said subject and wherein said determination comprises the use of a capture-binder that binds specifically to full-length DPP3, wherein said capture-binder is immobilized on a surface.

Another embodiment of the present application relates to a method for the prediction of an increase of DPP3 in a critically ill patient, wherein said level of DPP3 is DPP3 activity, wherein the method for determining DPP3 activity in a bodily fluid sample of said subject comprises the steps:
contacting said sample with a capture-binder that binds specifically to full-length DPP3,
separating DPP3 bound to said capture binder,
adding substrate of DPP3 to said separated DPP3,
quantifying of said DPP3 activity by measuring and quantifying the conversion of a substrate of DPP3.

Another embodiment of the present application relates to a method for therapy guidance and/or therapy monitoring and/or therapy stratification in a critically ill patient, wherein the amount of DPP3 protein and/or DPP3 activity is determined in a bodily fluid sample of said subject and wherein said separation step is a washing step that removes ingredients of the sample that are not bound to said capture-binder from the captured DPP3.

Another specific embodiment of the present application relates to a method for the prediction of an increase of DPP3 in a critically ill patient, wherein the DPP3 activity is determined in a bodily fluid sample of said subject and wherein DPP3 substrate conversion is detected by a method selected from the group comprising: fluorescence of fluorogenic substrates (e.g. Arg-Arg-βNA, Arg-Arg-AMC), color change of chromogenic substrates, luminescence of substrates coupled to aminoluciferin (Promega Protease-Glo™ Assay), mass spectrometry, HPLC/FPLC (reversed phase chromatography, size exclusion chromatography), thin layer chromatography, capillary zone electrophoresis, gel electrophoresis followed by activity staining (immobilized, active DPP3) or western blot (cleavage products).

Another preferred embodiment of the present application relates to a method for the prediction of an increase of DPP3 in a critically ill patient, wherein the DPP3 activity is determined in a bodily fluid sample of said subject and wherein said substrate may be selected from the group comprising: angiotensin II, III and IV, Leu-enkephalin, Met-enkephalin, endomorphin 1 and 2, valorphin, β-casomorphin, dynorphin, proctolin, ACTH and MSH, or di-peptides coupled to a fluorophore, a chromophore or aminoluciferin wherein the di-peptide is Arg-Arg.

Another embodiment of the present application relates to a method for the prediction of an increase of DPP3 in a critically ill patient, wherein the DPP3 activity is determined in a bodily fluid sample of said subject and wherein said substrate may be selected from the group comprising: A di-peptide coupled to a fluorophore, a chromophore or aminoluciferin wherein the di-peptide is Arg-Arg.

Another embodiment of the present application relates to a method for the prediction of an increase of DPP3 in a critically ill patient, wherein the sample of bodily fluid of said patient is selected from the group of whole blood, serum and plasma.

The level of DPP3 as the amount of DPP3 protein and/or DPP3 activity in a sample of bodily fluid of said subject may be determined by different methods, e.g., immunoassays, activity assays, mass spectrometric methods etc.

DPP3 activity can be measured by detection of cleavage products of DPP3 specific substrates. Known peptide hormone substrates include Leu-enkephalin, Met-enkephalin, endomorphin 1 and 2, valorphin, β-casomorphin, dynorphin, proctolin, ACTH (Adrenocorticotropic hormone) and MSH (melanocyte-stimulating hormone; Abramić et al. 2000, Barsun et al. 2007, Dhanda et al. 2008). The cleavage of mentioned peptide hormones as well as other untagged oligopeptides (e.g. Ala-Ala-Ala-Ala, Dhanda et al. 2008) can be monitored by detection of the respective cleavage products. Detection methods include, but are not limited to, HPLC analysis (e.g. Lee & Snyder 1982), mass spectrometry (e.g. Abramić et al. 2000), H1-NMR analysis (e.g. Vandenberg et al. 1985), capillary zone electrophoresis (CE; e.g. Baršun et al. 2007), thin layer chromatography (e.g. Dhanda et al. 2008) or reversed phase chromatography (e.g. Mazocco et al. 2006).

Detection of fluorescence due to hydrolysis of fluorogenic substrates by DPP3 is a standard procedure to monitor DPP3 activity. Those substrates are specific di- or tripeptides (Arg-Arg, Ala-Ala, Ala-Arg, Ala-Phe, Asp-Arg, Gly-Ala, Gly-Arg, Gly-Phe, Leu-Ala, Leu-Gly, Lys-Ala, Phe-Arg, Suc-Ala-Ala-Phe) coupled to a fluorophore. Fluorophores include but are not limited to β-naphtylamide (2-naphtylamide, βNA, 2NA), 4-methoxy-β-naphtylamide (4-methoxy-2-naphtylamide) and 7-amido-4-methylcoumarin (AMC, MCA; Abramić et al. 2000, Ohkubo et al. 1999). Cleavage of these fluorogenic substrates leads to the release of fluorescent β-naphtylamine or 7-amino-4-methylcoumarin respectively. In a liquid phase assay or an ECA substrate and DPP3 are incubated in for example a 96 well plate format and fluorescence is measured using a fluorescence detector (Ellis & Nuenke 1967). Additionally, DPP3 carrying samples can be immobilized and divided on a gel by electrophoresis, gels stained with fluorogenic substrate (e.g. Arg-Arg-ONA) and Fast Garnet GBC and fluorescent protein bands detected by a fluorescence reader (Ohkubo et al. 1999). The same peptides (Arg-Arg, Ala-Ala, Ala-Arg, Ala-Phe, Asp-Arg, Gly-Ala, Gly-Arg, Gly-Phe, Leu-Ala, Leu-Gly, Lys-Ala, Phe-Arg, Suc-Ala-Ala-Phe) can be coupled to chromophores, such Asp-nitroanilide diacetate. Detection of color change due to hydrolysis of chromogenic substrates can be used to monitor DPP3 activity.

Another option for the detection of DPP3 activity is a Protease-Glo™ Assay (commercially available at Promega). In this embodiment of said method DPP3 specific di- or tripeptides (Arg-Arg, Ala-Ala, Ala-Arg, Ala-Phe, Asp-Arg, Gly-Ala, Gly-Arg, Gly-Phe, Leu-Ala, Leu-Gly, Lys-Ala, Phe-Arg, Suc-Ala-Ala-Phe) are coupled to aminoluciferin. Upon cleavage by DPP3, aminoluciferin is released and serves as a substrate for a coupled luciferase reaction that emits detectable luminescence.

In a preferred embodiment DPP3 activity is measured by addition of the fluorogenic substrate Arg-Arg-βNA and monitoring fluorescence in real time.

In a specific embodiment of said method for determining active DPP3 in a bodily fluid sample of a subject said capture binder reactive with DPP3 is immobilized on a solid phase.

The test sample is passed over the immobile binder, and DPP3, if present, binds to the binder and is itself immobilized for detection. A substrate may then be added, and the reaction product may be detected to indicate the presence or amount of DPP3 in the test sample. For the purposes of the present description, the term “solid phase” may be used to include any material or vessel in which or on which the assay may be performed and includes, but is not limited to: porous materials, nonporous materials, test tubes, wells, slides, agarose resins (e.g. Sepharose from GE Healthcare Life Sciences), magnetic particals (e.g. Dynabeads™ or Pierce™ magnetic beads from Thermo Fisher Scientific), etc.

In another embodiment of the invention, the level of DPP3 is determined by contacting said sample of bodily fluid with a capture binder that binds specifically to DPP3.

In another preferred embodiment of the invention, said capture binder for determining the level of DPP3 may be selected from the group of antibody, antibody fragment or non-IgG scaffold.

In a specific embodiment of the invention, said capture binder is an antibody.

The level of DPP3 may be the amount of DPP3 protein and/or DPP3 activity in a sample of bodily fluid of said subject and may be determined for example by one of the following methods:
1. Luminescence immunoassay for the quantification of DPP3 protein concentrations (LIA) (Rehfeld et al., 2019 JALM 3(6): 943-953).

The LIA is a one-step chemiluminescence sandwich immunoassay that uses white high-binding polystyrene microtiter plates as solid phase. These plates are coated with monoclonal anti-DPP3 antibody AK2555 (capture antibody). The tracer anti-DPP3 antibody AK2553 is labeled with MA70-acridinium-NHS-ester and used at a concentration of 20 ng per well. Twenty microliters of samples (e.g., serum, heparin-plasma, citrate-plasma or EDTA-plasma derived from patients' blood) and calibrators are pipetted into coated white microtiter plates. After adding the tracer antibody AK2553, the microtiter plates are incubated for 3 h at room temperature and 600 rpm. Unbound tracer is then removed by 4 washing steps (350 μL per well). Remaining chemiluminescence is measured for is per well by using a microtiter plate luminometer. The concentration of DPP3 is determined with a 6-point calibration curve. Calibrators and samples are preferably run in duplicate.
2. Enzyme capture activity assay for the quantification of DPP3 activity (ECA) (Rehfeld et al., 2019 JALM 3(6): 943-953).

The ECA is a DPP3-specific activity assay that uses black high-binding polystyrene microtiter plates as solid phase. These plates are coated with monoclonal anti-DPP3 antibody AK2555 (capture antibody). Twenty microliters of samples (e.g., serum, heparin-plasma, citrate-plasma, EDTA-plasma) and calibrators are pipetted into coated black microtiter plates. After adding assay buffer (200 μL), the microtiter plates are incubated for 2 h at 22° C. and 600 rpm. DPP3 present in the samples is immobilized by binding to the capture antibody. Unbound sample components are removed by 4 washing steps (350 μL per well). The specific activity of immobilized DPP3 is measured by the addition of the fluorogenic substrate, Arg-Arg-β-Naphthylamide (Arg2-βNA), in reaction buffer followed by incubation at 37° C. for 1 h. DPP3 specifically cleaves Arg2-βNA into Arg-Arg dipeptide and fluorescent β-naphthylamine. Fluorescence is measured with a fluorometer using an excitation wavelength of 340 nm and emission is detected at 410 nm. The activity of DPP3 is determined with a 6-point calibration curve. Calibrators and samples are preferably run in duplicates.
3. Liquid-phase assay for the quantification of DPP3 activity (LAA) (modified from Jones et al., Analytical Biochemistry, 1982).

The LAA is a liquid phase assay that uses black non-binding polystyrene microtiter plates to measure DPP3 activity. Twenty microliters of samples (e.g. serum, heparin-plasma, citrate-plasma) and calibrators are pipetted into non-binding black microtiter plates. After addition of fluorogenic substrate, Arg2-ONA, in assay buffer (200 μL), the initial βNA fluorescence (T=0) is measured in a fluorimeter using an excitation wavelength of 340 nm and emission is detected at 410 nm. The plate is then incubated at 37° C. for 1 hour. The final fluorescence of (T=60) is measured. The difference between final and initial fluorescence is calculated. The activity of DPP3 is determined with a 6-point calibration curve. Calibrators and samples are preferably run in duplicates.

In a specific embodiment an assay is used for determining the level of DPP3, wherein the assay sensitivity of said assay is able to quantify the DPP3 of healthy subjects and is <20 ng/ml, preferably <30 ng/ml and more preferably <40 ng/ml.

In a specific embodiment, said binder exhibits a binding affinity to DPP3 of at least 10⁷ M⁻¹, preferred 10⁸ M⁻¹, more preferred affinity is greater than 10⁹ M⁻¹, most preferred greater than 10¹⁰ M⁻¹. A person skilled in the art knows that it may be considered to compensate lower affinity by applying a higher dose of compounds and this measure would not lead out-of-the-scope of the invention.

A bodily fluid according to the present invention is in one particular embodiment a blood sample. A blood sample may be selected from the group comprising whole blood, serum and plasma. In a specific embodiment of the method said sample is selected from the group comprising human citrate plasma, heparin plasma and EDTA plasma.

In one embodiment such assay for determining the level of DPP3 is a sandwich immunoassay using any kind of detection technology including but not restricted to enzyme label, chemiluminescence label, electrochemiluminescence label, preferably a fully automated assay. In one embodiment of the diagnostic method such an assay is an enzyme labeled sandwich assay. Examples of automated or fully automated assay comprise assays that may be used for one of the following systems: Roche Elecsys®, Abbott Architect®, Siemens Centauer®, Brahms Kryptor®, BiomerieuxVidas®, Alere Triage®.

A variety of immunoassays are known and may be used for the assays and methods of the present invention, these include: mass spectrometry (MS), luminescence immunoassay (LIA), radioimmunoassays (“RIA”), homogeneous enzyme-multiplied immunoassays (“EMIT”), enzyme linked immunoadsorbent assays (“ELISA”), apoenzyme reactivation immunoassay (“ARIS”), luminescence-based bead arrays, magnetic beads based arrays, protein microarray assays, rapid test formats such as for instance dipstick immunoassays, immuno-chromatographic strip tests, rare cryptate assay and automated systems/analysers.

In one embodiment of the invention, it may be a so-called POC-test (point-of-care) that is a test technology, which allows performing the test within less than 1 hour near the patient without the requirement of a fully automated assay system. One example for this technology is the immunochromatographic test technology, e.g. a microfluidic device.

In a preferred embodiment said label is selected from the group comprising chemiluminescent label, enzyme label, fluorescence label, radioiodine label.

The assays can be homogenous or heterogeneous assays, competitive and non-competitive assays. In one embodiment, the assay is in the form of a sandwich assay, which is a non-competitive immunoassay, wherein the molecule to be detected and/or quantified is bound to a first antibody and to a second antibody. The first antibody may be bound to a solid phase, e.g., a bead, a surface of a well or other container, a chip or a strip, and the second antibody is an antibody which is labeled, e.g., with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD, Oxford; 3rd ed. (May 2005), ISBN-13: 978-0080445267; Hultschig C et al., Curr Opin Chem Biol. 2006 February; 10(1):4-10. PMID: 16376134).

In another embodiment the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

In another embodiment, said labeling system comprises rare earth cryptates or rare earth chelates in combination with fluorescence dye or chemiluminescence dye, in particular a dye of the cyanine type.

In the context of the present invention, fluorescence based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

In the context of the present invention, chemiluminescence based assays comprise the use of dyes, based on the physical principles described for chemiluminescent materials in (Kirk-Othmer, Encyclopedia of chemical technology, 4th ed., executive editor, J. L Kroschwitz; editor, M. Howe-Grant, John Wiley & Sons, 1993, vol. 15, p. 518-562, incorporated herein by reference, including citations on pages 551-562). Preferred chemiluminescent dyes are acridinium esters.

As mentioned herein, an “assay” or “diagnostic assay” can be of any type applied in the field of diagnostics. Such an assay may be based on the binding of an analyte to be detected to one or more capture probes with a certain affinity. Concerning the interaction between capture molecules and target molecules or molecules of interest, the affinity constant is preferably greater than 10⁸ M⁻¹.

In a specific embodiment at least one of said two binders is labeled in order to be detected.

The ADM-NH₂ levels of the present invention have been determined with the described ADM-NH₂ assay (Weber et al. 2017. JALM 2(2):1-4). The DPP3 levels of the present invention have been determined with the described DPP3-assays as outlined in the examples (Rehfeld et al. 2019. JALM 3(6): 943-953). The mentioned threshold values above might be different in other assays, if these have been calibrated differently from the assay systems used in the present invention. Therefore, the mentioned cut-off values above shall apply for such differently calibrated assays accordingly, taking into account the differences in calibration. One possibility of quantifying the difference in calibration is a method comparison analysis (correlation) of the assay in question with the respective biomarker assay used in the present invention by measuring the respective biomarker (e.g., bio-ADM, DPP3) in samples using both methods. Another possibility is to determine with the assay in question, given this test has sufficient analytical sensitivity, the median biomarker level of a representative normal population, compare results with the median biomarker levels as described in the literature and recalculate the calibration based on the difference obtained by this comparison. With the calibration used in the present invention, samples from normal (healthy) subjects have been measured: median plasma bio-ADM (mature ADM-NH₂) was 24.7 μg/ml, the lowest value 11 μg/ml and the 99^{th} percentile 43 μg/ml (Marino et al. 2014. Critical Care 18:R34). With the calibration used in the present invention, samples from 5,400 normal (healthy) subjects (swedish single-center prospective population-based Study (MPP-RES)) have been measured: median (interquartile range) plasma DPP3 was 14.5 ng/ml (11.3 ng/ml-19 ng/ml). It was shown that DPP3 concentrations strongly correlate with DPP3 activity in the blood (Rehfeld et al. 2019. J Appl Lab Med 3: 943-953; Deniau et al. 2019. Eur J Heart Fail 22: 290-299). Consequently, the level of active DPP3 may be determined using respective thresholds and threshold ranges that correspond to the threshold and threshold ranges used by determining the level of DPP3 protein.

In a preferred embodiment, the treatment is initiated or changed immediately upon provision of the result of the sample analysis indicating the level of DPP3 in the sample. In further embodiments, the treatment may be initiated within 12 hours, preferably 6, 4, 2, 1, 0.5, 0.25 hours or immediately after receiving the result of the sample analysis.

In certain embodiments of the present invention, the increase of DPP3 in said patient is during follow-up time.

In certain embodiments of the present invention, said follow-up time is up to 12 hours, preferably up to 24, 48, 72, 96 hours, more preferred up to 5 days, even more preferred up to 6 days, most preferred up to 7 days.

In certain embodiments of the present invention, the increase of DPP3 in said patient is within 12 hours, preferably up to 24, 48, 72, 96 hours, more preferred up to 5 days, even more preferred up to 6 days, most preferred up to 7 days. In other embodiments of the present invention, said follow-up time is 2 days, or 3 days, or 7 days.

In certain embodiments of the present invention, the increase of DPP3 in said patient is within up to 12 hours, preferably up to 24, 48, 72, 96 hours, more preferred up to 5 days, even more preferred up to 6 days, most preferred up to 7 days.

In other embodiments of the present invention, the increase of DPP3 in said patient is within 2 days, or 3 days, or 7 days.

The present invention further relates to a kit for carrying out the method of the invention, comprising detection reagents for determining DPP3 in a sample from a patient, and reference data, such as a reference and/or threshold level, corresponding to a level of DPP3 in said sample between 22 and 40 ng/mL, wherein said reference data is preferably stored on a computer readable medium and/or employed in the form of computer executable code configured for comparing the determined DPP3 to said reference data.

In one embodiment of the method described herein, the method additionally comprises comparing the determined level of DPP3 in patients with shock or patients running into shock to a reference and/or threshold level, wherein said comparing is carried out in a computer processor using computer executable code.

The methods of the present invention may in part be computer-implemented. For example, the step of comparing the detected level of DPP3 with a reference and/or threshold level can be performed in a computer system. For example, the determined values may be entered (either manually by a health professional or automatically from the device(s) in which the respective marker level(s) has/have been determined) into the computer-system. The computer-system can be directly at the point-of-care (e.g., primary care unit or ED) or it can be at a remote location connected via a computer network (e.g., via the internet, or specialized medical cloud-systems, optionally combinable with other IT-systems or platforms such as hospital information systems (HIS)). Alternatively, or in addition, the associated therapy guidance and/or therapy stratification will be displayed and/or printed for the user (typically a health professional such as a physician).

The term “critically ill patients” refers to patients suffering from an acute disease or acute condition, e.g., an intensive care unit (ICU) patient who requires constant and/or intense observation of the patient's health state. Said patient may suffer from severe infection, sepsis, heart failure, chronic heart failure, acute heart failure (AHF), myocardial infarction (MI), stroke, organ dysfunction or organ failure (e.g., dysfunction or failure of liver, kidney, lung), undergo major surgery, trauma (e.g., burn trauma, polytrauma), shock and/or running into shock. In other embodiments, said patient may suffer from severe infection, sepsis, heart failure, chronic heart failure, acute heart failure (AHF), myocardial infarction (MI), stroke, organ dysfunction or organ failure (e.g., dysfunction or failure of liver, kidney, lung), undergo major surgery, trauma (e.g., burn trauma, polytrauma), shock and/or running into shock, or ARDS.

Patients with chronic heart failure (HF) may include patients with worsening signs and symptoms of chronic heart failure and acute decompensation of chronic heart failure.

Chronic HF with worsening signs and symptoms is in particular characterized by:
(i) the presence of structural or functional failure of the heart that impairs its ability to supply sufficient blood flow to meet body's needs,
(ii) volume overload (manifested by pulmonary and/or systemic congestion) and/or profound depression of cardiac output (manifested by hypotension, renal insufficiency and/or a shock syndrome) and whereas the patient is not in need of urgent therapy and does not require hospitalization, but is in need of therapy adjustment.

Chronic heart failure may also decompensate (termed acute decompensated heart failure or acute decompensated chronic heart failure), which is most commonly the result from an intercurrent illness (such as pneumonia), myocardial infarction, arrhythmias, uncontrolled hypertension or a patient's failure to maintain fluid restriction, diet or medication.

New onset acute HF and acute decompensated chronic HF are characterized by:
(i) the presence of structural or functional failure of the heart that impairs its ability to supply sufficient blood flow to meet body's needs,
(ii) volume overload (manifested by pulmonary and/or systemic congestion) and/or profound depression of cardiac output (manifested by hypotension, renal insufficiency and/or a shock syndrome) and whereas the patient is in need of urgent therapy or therapy adjustment and does require hospitalization.

| Acute HF | | Chronic HF | |
|---|---|---|---|
| New-onset | Acute decompensated HF = | Worsening signs/ | Stable |
| AHF | acute decompensated | symptoms of | chronic HF |
| | chronic HF | chronic HF | |

The above definitions of acute heart failure that either new-onset AHF or acute decompensated HF or acute decompensated chronic HF or worsening signs/symptoms of chronic heart failure are in line with Voors et al., European Journal of Heart Failure (2016), 18, 716-726.

The patient described herein can be at the emergency department (ED) or intensive care unit (ICU), or in other point of care settings, such as in an emergency transporter, such as an ambulance, or at a general practitioner, who is confronted with a patient with one of the diseases detailed herein.

The term “ICU-patient” relates, without limitation, to a patient who has been admitted to an intensive care unit. An intensive care unit, which can also be termed an intensive therapy unit or intensive treatment unit (ITU) or critical care unit (CCU), is a special department of a hospital or health care facility that provides intensive treatment medicine. ICU-patients usually suffer from severe and life-threatening illnesses and injuries, which require constant, close monitoring and support from specialist equipment and/or medications in order to ensure normal bodily functions. Common conditions that are treated within ICUs include, without limitation, acute respiratory distress syndrome (ARDS), trauma, organ dysfunction or organ failure, sepsis and shock.

In another specific embodiment of the invention, said shock is selected from the group comprising shock due to hypovolemia, cardiogenic shock, obstructive shock and distributive shock, in particular cardiogenic or septic shock.

In another specific embodiment of the invention, said shock is selected from the group comprising:
in case of cardiogenic shock said patient has suffered an acute coronary syndrome (e.g., acute myocardial infarction) or has heart failure (e.g., acute decompensated heart failure), myocarditis, arrhythmia, cardiomyopathy, valvular heart disease, aortic dissection with acute aortic stenosis, traumatic chordal rupture or massive pulmonary embolism, or
in case of hypovolemic shock said patient may have suffered a hemorrhagic disease including gastrointestinal bleed, trauma, vascular etiologies (e.g., ruptured abdominal aortic aneurysm, tumor eroding into a major blood vessel) and spontaneous bleeding in the setting of anticoagulant use or a non-hemorrhagic disease including vomiting, diarrhea, renal loss, skin losses/insensible losses (e.g., burns, heat stroke) or third-space loss in the setting of pancreatitis, cirrhosis, intestinal obstruction, trauma, or
in case of obstructive shock said patient may have suffered a cardiac tamponade, tension pneumothorax, pulmonary embolism or aortic stenosis, or
in case of distributive shock said patient has septic shock, neurogenic shock, anaphylactic shock or shock due to adrenal crisis.

Shock is characterized by decreased oxygen delivery and/or increased oxygen consumption or inadequate oxygen utilization leading to cellular and tissue hypoxia. It is a life-threatening condition of circulatory failure and most commonly manifested as hypotension (systolic blood pressure less than 90 mm Hg or MAP less than 65 mmHg). Shock is divided into four main types based on the underlying cause: hypovolemic, cardiogenic, obstructive, and distributive shock (Vincent and De Backer 2014. N. Engl. J. Med. 370(6): 583).

Hypovolemic shock is characterized by decreased intravascular volume and can be divided into two broad subtypes: hemorrhagic and non-hemorrhagic. Common causes of hemorrhagic hypovolemic shock include gastrointestinal bleed, trauma, vascular etiologies (e.g., ruptured abdominal aortic aneurysm, tumor eroding into a major blood vessel) and spontaneous bleeding in the setting of anticoagulant use. Common causes of non-hemorrhagic hypovolemic shock include vomiting, diarrhea, renal loss, skin losses/insensible losses (e.g., burns, heat stroke) or third-space loss in the setting of pancreatitis, cirrhosis, intestinal obstruction, trauma. For review see Kova and Paul 2018. Shock. StatPearls [Internet]. Treasure Island (FL): StatPearls Publishing; 2019-2018 Oct. 27.

Cardiogenic shock (CS) is defined as a state of critical endorgan hypoperfusion due to reduced cardiac output. Notably, CS forms a spectrum that ranges from mild hypoperfusion to profound shock. Established criteria for the diagnosis of CS are: (i) systolic blood pressure, ≤90 mmHg for >30 min or vasopressors required to achieve a blood pressure ≥90 mmHg; (ii) pulmonary congestion or elevated left-ventricular filling pressures; (iii) signs of impaired organ perfusion with at least one of the following criteria: (a) altered mental status; (b) cold, clammy skin; (c) oliguria (<0.5 mL/kg/h or <30 mL/h); (d) increased serum-lactate (Revnolds and Hochman 2008. Circulation 117: 686-697). Acute myocardial infarction (AMI) with subsequent ventricular dysfunction is the most frequent cause of CS accounting for approximately 80% of cases. Mechanical complications such as ventricular septal (4%) or free wall rupture (2%), and acute severe mitral regurgitation (7%) are less frequent causes of CS after AMI. (Hochman et al. 2000. J Am Coll Cardiol 36: 1063-1070). Non-AMI-related CS may be caused by decompensated valvular heart disease, acute myocarditis, arrhythmias, etc. with heterogeneous treatment options. This translates in 40 000 to 50 000 patients per year in the USA and 60 000 to 70 000 in Europe. Despite advances in treatment mainly by early revascularization with subsequent mortality reduction, CS remains the leading cause of death in AMI with mortality rates still approaching 40-50% according to recent registries and randomized trials (Goldberg et al. 2009. Circulation 119: 1211-1219).

Obstructive shock is due to a physical obstruction of the great vessels or the heart itself. Several conditions can result in this form of shock (e.g. cardiac tamponade, tension pneumothorax, pulmonary embolism, aortic stenosis). For review see Kova and Paul 2018. Shock. StatPearls [Internet]. Treasure Island (FL): StatPearls Publishing; 2019-2018 Oct. 27.

According to the cause, there are four types of distributive shock: neurogenic shock (decreased sympathetic stimulation leading to decreased vasal tone), anaphylactic shock, septic shock and shock due to adrenal crisis. In addition to sepsis, distributive shock can be caused by systemic inflammatory response syndrome (SIRS) due to conditions other than infection such as pancreatitis, burns or trauma. Other causes include, toxic shock syndrome (TSS), anaphylaxis (a sudden, severe allergic reaction), adrenal insufficiency (acute worsening of chronic adrenal insufficiency, destruction or removal of the adrenal glands, suppression of adrenal gland function due to exogenous steroids, hypopituitarism and metabolic failure of hormone production), reactions to drugs or toxins, heavy metal poisoning, hepatic (liver) insufficiency and damage to the central nervous system. For review see Kova and Paul 2018. Shock. StatPearls [Internet]. Treasure Island (FL): StatPearls Publishing; 2019-2018 Oct. 27.

Refractory shock has been defined as requirement of noradrenaline infusion of >0.5 μg/kg/min despite adequate volume resuscitation. Mortality in these patients may be as high as 94% and the assessment and management of these patients requires a much more aggressive approach for survival. The term “refractory shock” is used when the tissue perfusion cannot be restored with the initial corrective measures employed (e.g. vasopressors) and may therefore be referred to as “high vasopressor-dependent” or “vasopressor-resistant” shock (Udupa and Shetty 2018. Indian J Respir Care 7: 67-72). Patients with refractory shock may have features of inadequate perfusion such as hypotension (mean arterial blood pressure <65 mmHg), tachycardia, cold peripheries, prolonged capillary refill time, and tachypnea consequent to the hypoxia and acidosis. Fever may be seen in septic shock. Other signs of hypoperfusion such as altered sensorium, hyperlactatemia, and oliguria may also be seen. These well-known signs of shock are not helpful in identifying whether the problem is at the pump (heart) or circuitry (vessels and tissues). Different types of shock can coexist, and all forms of shock can become refractory, as evidenced by unresponsiveness to high-dose vasopressors (Udupa and Shetty 2018. Indian J Respir Care 7: 67-72).

Septic shock is a potentially fatal medical condition that occurs when sepsis, which is organ injury or damage in response to infection, leads to dangerously low blood pressure and abnormalities in cellular metabolism. The Third International Consensus Definitions for Sepsis and Septic Shock (Sepsis-3) defines septic shock as a subset of sepsis in which particularly profound circulatory, cellular, and metabolic abnormalities are associated with a greater risk of mortality than with sepsis alone. Patients with septic shock can be clinically identified by a vasopressor requirement to maintain a mean arterial pressure of 65 mm Hg or greater and serum lactate level greater than 2 mmol/L (>18 mg/dL) in the absence of hypovolemia. This combination is associated with hospital mortality rates greater than 40% (Singer et al. 2016. JAMA. 315 (8): 801-10). The primary infection is most commonly caused by bacteria, but also may be by fungi, viruses or parasites. It may be located in any part of the body, but most commonly in the lungs, brain, urinary tract, skin or abdominal organs. It can cause multiple organ dysfunction syndrome (formerly known as multiple organ failure) and death. Frequently, people with septic shock are cared for in intensive care units. It most commonly affects children, immunocompromised individuals, and the elderly, as their immune systems cannot deal with infection as effectively as those of healthy adults. The mortality rate from septic shock is approximately 25-50%.

In one embodiment of the invention said patient is a critically ill patient having shock or running into shock at the time the sample of bodily fluid of said patient is taken.

“A patient running into shock” is defined as a critically ill patient that does not have shock at the time the bodily fluid is taken from said patient, but has an increased risk of developing shock.

In a specific embodiment said shock is septic shock or cardiogenic shock.

The term “therapy” or “treatment” refers to critical care medicaments. Said critical care medicaments are selected from the group comprising alkaline phosphatase, immune suppressors, corticosteroids, vasopressors, fluids, anti-ADM antibodies, anti-ADM antibody fragments, anti-ADM scaffolds, anti-DPP3 inhibitors (e.g., anti-DPP3 antibodies, anti-DPP3 antibody fragments, anti-DPP3 scaffolds).

Another preferred embodiment of the present application is further a method for the prediction of an increase of DPP3 in a critically ill patient, wherein a patient is selected for therapy/treatment if the level of a DPP3 in said sample is below said pre-determined threshold, wherein said therapy is selected from the group of alkaline phosphatase, immune suppressors, corticosteroids, vasopressors, fluids, anti-ADM antibodies or antibody fragments.

Another preferred embodiment of the present application is further a method for the prediction of an increase of DPP3 in a critically ill patient, wherein said anti-ADM antibodies or with anti-ADM antibody fragments or anti-ADM scaffolds are directed to the N-terminal part (amino acids 1-21) of ADM: YRQSMNNFQGLRSFGCRFGTC (SEQ ID No. 14).

Another embodiment of the present application relates to a method for the prediction of an increase of DPP3 in a critically ill patient, wherein said anti-ADM antibody or anti-ADM antibody fragment recognizes and binds to the N-terminal end (amino acid 1) of ADM.

A further embodiment of the present application relates to a method for the prediction of an increase of DPP3 in a critically ill patient, wherein said antibody, antibody fragment or non-Ig scaffold does not bind to the C-terminal portion of ADM, having the sequence amino acid 43-52 of ADM: PRSKISPQGY-NH₂(SEQ ID NO: 24).

Another embodiment of the present application relates to a method for the prediction of an increase of DPP3 in a critically ill patient, wherein said antibody or fragment is a monoclonal antibody or fragment that binds to ADM or an antibody fragment thereof, wherein the heavy chain comprises the sequences:

CDR1:
SEQ ID NO: 1
GYTFSRYW
CDR2:
SEQ ID NO: 2
ILPGSGST
CDR3:
SEQ ID NO: 3
TEGYEYDGFDY

and wherein the light chain comprises the sequences:

CDR1:
SEQ ID NO: 4
QSIVYSNGNTY
CDR2:
RVS
CDR3:
SEQ ID NO: 5
FQGSHIPYT.

Another embodiment of the present application relates to a method for the prediction of an increase of DPP3 in a critically ill patient, wherein said antibody or fragment comprises a sequence selected from the group comprising as a VH region:

SEQ ID NO: 6
(AM-VH-C)
SEQ ID NO: 7
(AM-VH1)
SEQ ID NO: 8
(AM-VH2-E40)
SEQ ID NO: 9
(AM-VH3-T26-E55)
SEQ ID NO: 10
(AM-VH4-T26-E40-E55)

and comprises a sequence selected from the group comprising the following sequence as a VL region:

SEQ ID NO: 11
(AM-VL-C)
SEQ ID NO: 12
(AM-VL1)
SEQ ID NO: 13
(AM-VL2-E40)

Another embodiment of the present application relates to a method for the prediction of an increase of DPP3 in a critically ill patient, wherein said antibody or fragment comprises the following sequence as a heavy chain:

SEQ ID NO: 32

[symbol]

[symbol]

indicates data missing or illegible when filed

or a sequence that is >95% identical to it,
and comprises the following sequence as a light chain:

SEQ ID NO: 33

[symbol]

[symbol]

indicates data missing or illegible when filed

or a sequence that is >95% identical to it.

A specific embodiment of the present invention relates to a method for the prediction of an increase of DPP3 in a critically ill patient, wherein said anti-ADM antibody or anti-ADM antibody fragment may be administered in a dose of at least 0.5 mg/kg body weight, particularly at least 1.0 mg/kg body weight, more particularly, from 1.0 to 20.0 mg/kg body weight, e.g., from 2.0 to 10 mg/kg body weight, from 2.0 to 8.0 mg/kg body weight, or from 2.0 to 5.0 mg/kg body weight.

The efficacy of non-neutralizing antibody targeted against the N-terminus of ADM was investigated in a survival study in CLP-induced sepsis in mice. Pre-treatment with the non-neutralizing antibody resulted in decreased catecholamine infusion rates, kidney dysfunction, and ultimately improved survival (Struck et al. 2013. Intensive Care Med Exp 1(1):22; Wagner et al. 2013. Intensive Care Med Exp 1(1):21).

Due to these positive results, a humanized version of an N-terminal anti-ADM antibody, named Adrecizumab, has been developed for further clinical development. Beneficial effects of Adrecizumab on vascular barrier function and survival were recently demonstrated in preclinical models of systemic inflammation and sepsis (Geven et al. 2018. Shock 50(6):648-654). In this study, pre-treatment with Adrecizumab attenuated renal vascular leakage in endotoxemic rats as well as in mice with CLP-induced sepsis, which coincided with increased renal expression of the protective peptide Ang-1 and reduced expression of the detrimental peptide vascular endothelial growth factor. Also, pre-treatment with Adrecizumab improved 7-day survival in CLP-induced sepsis in mice from 10 to 50% for single and from 0 to 40% for repeated dose administration. Moreover, in a phase I study, excellent safety and tolerability was demonstrated (see Example 6): no serious adverse events were observed, no signal of adverse events occurring more frequently in Adrecizumab-treated subjects was detected and no relevant changes in other safety parameters were found (Geven et al. 2017. Intensive Care Med Exp 5 (Suppl 2) 0427). Of particular interest is the proposed mechanism of action of Adrecizumab. Both, animal and human data reveal a potent, dose-dependent increase of circulating ADM following administration of this antibody. Based on pharmacokinetic data and the lack of an increase in MR-proADM (an inactive peptide fragment derived from the same prohormone as ADM), the higher circulating ADM levels cannot be explained by an increased production.

A mechanistic explanation for this increase could be that the excess of antibody in the circulation may drain ADM from the interstitium to the circulation, since ADM is small enough to cross the endothelial barrier, whereas the antibody is not (Geven et al. 2018. Shock. 50(2):132-140). In addition, binding of the antibody to ADM leads to a prolongation of ADM's half-life. Even though NT-ADM antibodies partially inhibit ADM-mediated signalling, a large increase of circulating ADM results in an overall “net” increase of ADM activity in the blood compartment, where it exerts beneficial effects on endothelial cells (ECs; predominantly barrier stabilization), whereas ADMs detrimental effects on vascular smooth muscle cells (VSMCs; vasodilation) in the interstitium are reduced.

Throughout the specification the “antibodies”, or “antibody fragments” or “non-Ig scaffolds” in accordance with the invention are capable to bind ADM, and thus are directed against ADM, and thus can be referred to as “anti-ADM antibodies”, “anti-ADM antibody fragments”, or “anti-ADM non-Ig scaffolds”.

The term “antibody” generally comprises monoclonal and polyclonal antibodies and binding fragments thereof, in particular Fc-fragments as well as so called “single-chain-antibodies” (Bird et al. 1988), chimeric, humanized, in particular CDR-grafted antibodies, and dia or tetrabodies (Holliger et al. 1993). Also comprised are immunoglobulin-like proteins that are selected through techniques including, for example, phage display to specifically bind to the molecule of interest contained in a sample. In this context the term “specific binding” refers to antibodies raised against the molecule of interest or a fragment thereof. An antibody is considered to be specific, if its affinity towards the molecule of interest or the aforementioned fragment thereof is at least preferably 50-fold higher, more preferably 100-fold higher, most preferably at least 1000-fold higher than towards other molecules comprised in a sample containing the molecule of interest. It is well known in the art how to make antibodies and to select antibodies with a given specificity.

In one embodiment of the invention the anti-ADM antibody or anti-ADM antibody fragment or anti-ADM non-Ig scaffold is monospecific.

Monospecific anti-ADM antibody or monospecific anti-adrenomedullin antibody fragment or monospecific anti-ADM non-Ig scaffold means that said antibody or antibody fragment or non-Ig scaffold binds to one specific region encompassing at least 5 amino acids within the target ADM. Monospecific anti-ADM antibody or monospecific anti-adrenomedullin antibody fragment or monospecific anti-ADM non-Ig scaffold are anti-ADM antibodies or anti-ADM antibody fragments or anti-ADM non-Ig scaffolds that all have affinity for the same antigen. Monoclonal antibodies are monospecific, but monospecific antibodies may also be produced by other means than producing them from a common germ cell.

Said anti-ADM antibody or antibody fragment binding to ADM or non-Ig scaffold binding to ADM may be a non-neutralizing anti-ADM antibody or antibody fragment binding to ADM or non-Ig scaffold binding to ADM.

In a specific embodiment said anti-ADM antibody, anti-ADM antibody fragment or anti-ADM non-Ig scaffold is a non-neutralizing antibody, fragment or non-Ig scaffold. A neutralizing anti-ADM antibody, anti-ADM antibody fragment or anti-ADM non-Ig scaffold would block the bioactivity of ADM to nearly 100%, to at least more than 90%, preferably to at least more than 95%.

In contrast, a non-neutralizing anti-ADM antibody, or anti-ADM antibody fragment or anti-ADM non-Ig scaffold blocks the bioactivity of ADM less than 100%, preferably to less than 95%, preferably to less than 90%, more preferred to less than 80% and even more preferred to less than 50%. This means that bioactivity of ADM is reduced to less than 100%, to 95% or less but not more, to 90% or less but not more, to 80% or less but not more, to 50% or less but not more. This means that the residual bioactivity of ADM bound to the non-neutralizing anti-ADM antibody, or anti-ADM antibody fragment or anti-ADM non-Ig scaffold would be more than 0%, preferably more than 5%, preferably more than 10%, more preferred more than 20%, more preferred more than 50%.

In this context (a) molecule(s), being it an antibody, or an antibody fragment or a non-Ig scaffold with “non-neutralizing anti-ADM activity”, collectively termed here for simplicity as “non-neutralizing” anti-ADM antibody, antibody fragment, or non-Ig scaffold, that e.g. blocks the bioactivity of ADM to less than 80%, is defined as
a molecule or molecules binding to ADM, which upon addition to a culture of an eukaryotic cell line, which expresses functional human recombinant ADM receptor composed of CRLR (calcitonin receptor like receptor) and RAMP3 (receptor-activity modifying protein 3), reduces the amount of cAMP produced by the cell line through the action of parallel added human synthetic ADM peptide, wherein said added human synthetic ADM is added in an amount that in the absence of the non-neutralizing antibody to be analyzed, leads to half-maximal stimulation of cAMP synthesis, wherein the reduction of cAMP by said molecule(s) binding to ADM takes place to an extent, which is not more than 80%, even when the non-neutralizing molecule(s) binding to ADM to be analyzed is added in an amount, which is 10-fold more than the amount, which is needed to obtain the maximal reduction of cAMP synthesis obtainable with the non-neutralizing antibody to be analyzed.

The same definition applies to the other ranges; 95%, 90%, 50% etc.

An antibody or fragment according to the present invention is a protein including one or more polypeptides substantially encoded by immunoglobulin genes that specifically binds an antigen. The recognized immunoglobulin genes include the kappa, lambda, alpha (IgA), gamma (IgG₁, IgG₂, IgG₃, IgG₄), delta (IgD), epsilon (IgE) and mu (IgM) constant region genes, as well as the myriad immunoglobulin variable region genes. Full-length immunoglobulin light chains are generally about 25 Kd or 214 amino acids in length.

Full-length immunoglobulin heavy chains are generally about 50 Kd or 446 amino acid in length. Light chains are encoded by a variable region gene at the NH₂-terminus (about 110 amino acids in length) and a kappa or lambda constant region gene at the COOH-terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)₂, as well as bifunctional hybrid antibodies and single chains (e.g., Lanzavecchia et al. 1987. Eur. J. Immunol. 17:105; Huston et al. 1988. Proc. Natl. Acad. Sci. U.S.A., 85:5879-5883; Bird et al. 1988. Science 242:423-426; Hood et al. 1984, Immunology, Benjamin, N.Y., 2nd ed.; Hunkapiller and Hood 1986 Nature 323:15-16). An immunoglobulin light or heavy chain variable region includes a framework region interrupted by three hypervariable regions, also called complementarity determining regions (CDR's) (see, Sequences of Proteins of Immunological Interest, E. Kabat et al. 1983, U.S. Department of Health and Human Services). As noted above, the CDRs are primarily responsible for binding to an epitope of an antigen. An immune complex is an antibody, such as a monoclonal antibody, chimeric antibody, humanized antibody or human antibody, or functional antibody fragment, specifically bound to the antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody, although other mammalian species can be used, or the variable region can be produced by molecular techniques. Methods of making chimeric antibodies are well known in the art, e.g., see U.S. Pat. No. 5,807,715. A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDR's. The acceptor framework of a humanized immunoglobulin or antibody may have a limited number of substitutions by amino acids taken from the donor framework. Humanized or other monoclonal antibodies can have additional conservative amino acid substitutions, which have substantially no effect on antigen binding or other immunoglobulin functions. Exemplary conservative substitutions are those such as gly, ala; val, ile, leu; asp, glu; asn, gln; ser, thr; lys, arg; and phe, tyr. Humanized immunoglobulins can be constructed by means of genetic engineering (e.g., see U.S. Pat. No. 5,585,089). A human antibody is an antibody wherein the light and heavy chain genes are of human origin. Human antibodies can be generated using methods known in the art. Human antibodies can be produced by immortalizing a human B cell secreting the antibody of interest. Immortalization can be accomplished, for example, by EBV infection or by fusing a human B cell with a myeloma or hybridoma cell to produce a trioma cell. Human antibodies can also be produced by phage display methods (see, e.g. WO91/17271; WO92/001047; WO92/20791), or selected from a human combinatorial monoclonal antibody library (see the Morphosys website). Human antibodies can also be prepared by using transgenic animals carrying a human immunoglobulin gene (for example, see WO93/12227; WO 91/10741).

Thus, the anti-ADM antibody may have the formats known in the art. Examples are human antibodies, monoclonal antibodies, humanized antibodies, chimeric antibodies, CDR-grafted antibodies. In a preferred embodiment antibodies according to the present invention are recombinantly produced antibodies as e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH3 domain; bivalent Fab or multivalent Fab, e.g. formed via multimerization with the aid of a heterologous domain, e.g. via dimerization of dHLX domains, e.g. Fab-dHLX-FSx2; F(ab′)₂-fragments, scFv-fragments, multimerized multivalent or/and multi-specificscFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulines and numerous others.

In addition to anti-ADM antibodies other biopolymer scaffolds are well known in the art to complex a target molecule and have been used for the generation of highly target specific biopolymers. Examples are aptamers, spiegelmers, anticalins and conotoxins. For illustration of antibody formats please see FIGS. 1a, 1b and 1c.

In a preferred embodiment the anti-ADM antibody format is selected from the group comprising Fv fragment, scFv fragment, Fab fragment, scFab fragment, F(ab)₂ fragment and scFv-Fc Fusion protein. In another preferred embodiment the antibody format is selected from the group comprising scFab fragment, Fab fragment, scFv fragment and bioavailability optimized conjugates thereof, such as PEGylated fragments. One of the most preferred formats is the scFab format.

Non-Ig scaffolds may be protein scaffolds and may be used as antibody mimics as they are capable to bind to ligands or antigens. Non-Ig scaffolds may be selected from the group comprising tetranectin-based non-Ig scaffolds (e.g. described in US2010/0028995), fibronectin scaffolds (e.g. described in EP 1 266 025; lipocalin-based scaffolds (e.g. described in WO 2011/154420); ubiquitin scaffolds (e.g. described in WO 2011/073214), transferrin scaffolds (e.g. described in US 2004/0023334), protein A scaffolds (e.g. described in EP 2 231 860), ankyrin repeat based scaffolds (e.g. described in WO 2010/060748), microproteins preferably microproteins forming a cysteine knot) scaffolds (e.g. described in EP 2314308), Fyn SH3 domain based scaffolds (e.g. described in WO 2011/023685) EGFR-A-domain based scaffolds (e.g. described in WO 2005/040229) and Kunitz domain based scaffolds (e.g. described in EP 1 941 867).

In one embodiment of the invention anti-ADM antibodies according to the present invention may be produced as outlined in Example 1 by synthesizing fragments of ADM as antigens. Thereafter, binder to said fragments are identified using the below described methods or other methods as known in the art.

Humanization of murine antibodies may be conducted according to the following procedure: For humanization of an antibody of murine origin the antibody sequence is analyzed for the structural interaction of framework regions (FR) with the complementary determining regions (CDR) and the antigen. Based on structural modelling an appropriate FR of human origin is selected and the murine CDR sequences are transplanted into the human FR. Variations in the amino acid sequence of the CDRs or FRs may be introduced to regain structural interactions, which were abolished by the species switch for the FR sequences. This recovery of structural interactions may be achieved by random approach using phage display libraries or via directed approach guided by molecular modelling (Almagro and Fransson 2008. Humanization of antibodies. Front Biosci. 2008 Jan. 1; 13:1619-33).

In a preferred embodiment the ADM antibody format is selected from the group comprising Fv fragment, scFv fragment, Fab fragment, scFab fragment, F(ab)₂ fragment and scFv-Fc Fusion protein. In another preferred embodiment the antibody format is selected from the group comprising scFab fragment, Fab fragment, scFv fragment and bioavailability optimized conjugates thereof, such as PEGylated fragments. One of the most preferred formats is scFab format.

In another preferred embodiment, the anti-ADM antibody, anti-ADM antibody fragment, or anti-ADM non-Ig scaffold is a full-length antibody, antibody fragment, or non-Ig scaffold.

In a preferred embodiment the anti-ADM antibody or an anti-ADM antibody fragment or anti-ADM non-Ig scaffold is directed to and can bind to an epitope of at least 5 amino acids in length contained in ADM.

In a more preferred embodiment, the anti-ADM antibody or an anti-ADM antibody fragment or anti-ADM non-Ig scaffold is directed to and can bind to an epitope of at least 4 amino acids in length contained in ADM.

In one specific embodiment of the invention said anti-ADM antibody or anti-ADM antibody fragment binding to adrenomedullin or anti-ADM non-Ig scaffold binding to adrenomedullin is not ADM-binding-Protein-1 (complement factor H).

In one specific embodiment of the invention said anti-ADM antibody or anti-ADM antibody fragment binding to adrenomedullin or anti-ADM non-Ig scaffold binding to adrenomedullin binds to a region of preferably at least 4, or at least 5 amino acids within the sequence of amino acid 1-21 of mature human ADM: YRQSMNNFQGLRSFGCRFGTC SEQ ID No.: 14.

In a preferred embodiment of the present invention said anti-ADM antibody or anti-ADM antibody fragment or anti-ADM non-Ig scaffold binds to a region or epitope of ADM that is located in the N-terminal part (amino acid 1-21) of adrenomedullin.

In another preferred embodiment said anti-ADM-antibody or anti-ADM antibody fragment or anti-ADM non-Ig scaffold recognizes and binds to a region or epitope within amino acids 1-14 of adrenomedullin: YRQSMNNFQGLRSF (SEQ ID No.: 25) that means to the N-terminal part (amino acid 1-14) of adrenomedullin.

In another preferred embodiment said anti-ADM-antibody or anti-ADM antibody fragment or anti-ADM non-Ig scaffold recognizes and binds to a region or epitope within amino acids 1-10 of adrenomedullin: YRQSMNNFQG (SEQ ID No.: 26); that means to the N-terminal part (amino acid 1-10) of adrenomedullin.

In another preferred embodiment said anti-ADM antibody or anti-ADM antibody fragment or anti-ADM non-Ig scaffold recognizes and binds to a region or epitope within amino acids 1-6 of adrenomedullin: YRQSMN (SEQ ID No.: 27); that means to the N-terminal part (amino acid 1-6) of adrenomedullin. As stated above said region or epitope comprises preferably at least 4 or at least 5 amino acids in length.

In another preferred embodiment said anti-ADM antibody or anti-ADM antibody fragment or anti-ADM non-Ig scaffold recognizes and binds to the N-terminal end (amino acid 1) of adrenomedullin. N-terminal end means that the amino acid 1, that is “Y” of SEQ ID No. 20, 14 or 23, respectively and is mandatory for binding. The antibody or fragment or scaffold would neither bind N-terminal extended nor N-terminal modified Adrenomedullin nor N-terminal degraded adrenomedullin. This means in another preferred embodiment said anti-ADM-antibody or anti-ADM antibody fragment or anti-ADM non-Ig scaffold binds only to a region within the sequence of mature ADM if the N-terminal end of ADM is free. In said embodiment the anti-ADM antibody or anti-ADM antibody fragment or non-Ig scaffold would not bind to a region within the sequence of mature ADM if said sequence is e.g. comprised within pro-ADM.

For the sake of clarity, the numbers in brackets for specific regions of ADM like “N-terminal part (amino acid 1-21)” is understood by a person skilled in the art that the N-terminal part of ADM consists of amino acids 1-21 of the mature ADM sequence.

In another specific embodiment pursuant to the invention the herein provided anti-ADM antibody or anti-ADM antibody fragment or anti-ADM non-Ig scaffold does not bind to the C-terminal portion of ADM, i.e. the amino acid 43-52 of ADM: PRSKISPQGY-NH₂ (SEQ ID No.: 24).

In one specific embodiment it is preferred to use an anti-ADM antibody or anti-ADM antibody fragment or anti-ADM non-Ig scaffold according to the present invention, wherein said anti-ADM antibody or said anti-ADM antibody fragment or anti-ADM non-Ig scaffold leads to an increase of the ADM level or ADM immunoreactivity in serum, blood, plasma of at least 10%, preferably at least 50%, more preferably >50%, most preferably >100%.

In one specific embodiment it is preferred to use an anti-ADM antibody or anti-ADM antibody fragment or anti-ADM non-Ig scaffold according to the present invention, wherein said anti-ADM antibody or said anti-ADM antibody fragment or anti-ADM non-Ig scaffold is an ADM stabilizing antibody or an ADM stabilizing antibody fragment or an ADM stabilizing non-Ig scaffold that enhances the half-life (t_{1/2}; half retention time) of adrenomedullin in serum, blood, plasma at least 10%, preferably at least 50%, more preferably >50%, most preferably >100%.

The half-life (half retention time) of ADM may be determined in human serum, blood or plasma in absence and presence of an ADM stabilizing antibody or an ADM stabilizing antibody fragment or an ADM stabilizing non-Ig scaffold, respectively, using an immunoassay for the quantification of ADM.

The following steps may be conducted:
ADM may be diluted in human citrate plasma in absence and presence of an ADM stabilizing antibody or an ADM stabilizing antibody fragment or an ADM stabilizing non-Ig scaffold, respectively, and may be incubated at 24° C.
Aliquots are taken at selected time points (e.g. within 24 hours) and degradation of ADM may be stopped in said aliquots by freezing at −20° C.
The quantity of ADM may be determined by a hADM immunoassay directly, if the selected assay is not influenced by the stabilizing antibody. Alternatively, the aliquot may be treated with denaturing agents (like HCl) and, after clearing the sample (e.g. by centrifugation) the pH can be neutralized and the ADM-quantified by an ADM immunoassay. Alternatively, non-immunoassay technologies (e.g. RP-HPLC) can be used for ADM-quantification.
The half-life of ADM is calculated for ADM incubated in absence and presence of an ADM stabilizing antibody or an ADM stabilizing antibody fragment or an ADM stabilizing non-Ig scaffold, respectively.
The enhancement of half-life is calculated for the stabilized ADM in comparison to ADM that has been incubated in absence of an ADM stabilizing antibody or an ADM stabilizing antibody fragment or an ADM stabilizing non-Ig scaffold.

A two-fold increase of the half-life of ADM is an enhancement of half-life of 100%. Half-life (half retention time) is defined as the period over which the concentration of a specified chemical or drug takes to fall to half its baseline concentration in the specified fluid or blood. An assay that may be used for the determination of the half-life (half retention time) of adrenomedullin in serum, blood, plasma is described in Example 3.

In a preferred embodiment said anti-ADM antibody, anti-ADM antibody fragment or anti-ADM non-Ig scaffold is a non-neutralizing antibody, fragment or scaffold. A neutralizing anti-ADM antibody, anti-ADM antibody fragment or anti-ADM non-Ig scaffold would block the bioactivity of ADM to nearly 100%, to at least more than 90%, preferably to at least more than 95%. In other words, this means that said non-neutralizing anti-ADM antibody, anti-ADM antibody fragment or anti-ADM non-Ig scaffold blocks the bioactivity of ADM to less than 100%, preferably less than 95% preferably less than 90%, In an embodiment wherein said non-neutralizing anti-ADM antibody, anti-ADM antibody fragment or anti-ADM non-Ig scaffold blocks the bioactivity of ADM to less than 95% an anti-ADM antibody, anti-ADM antibody fragment or anti-ADM non-Ig scaffold that would block the bioactivity of ADM to more than 95% would be outside of the scope of said embodiment. This means in one embodiment that the bioactivity is reduced to 95% or less but not more, preferably to 90% or less, more preferably to 80% or less, more preferably to 50% or less but not more.

In one embodiment of the invention the non-neutralizing antibody is an antibody binding to a region of at least 5 amino acids within the sequence of amino acid 1-21 of mature human ADM (SEQ ID No.: 14), or an antibody binding to a region of at least 5 amino acids within the sequence of amino acid 1-19 of mature murine ADM (SEQ ID No.: 17).

In another preferred embodiment of the invention the non-neutralizing antibody is an antibody binding to a region of at least 4 amino acids within the sequence of amino acid 1-21 of mature human ADM (SEQ ID No.: 14), or an antibody binding to a region of at least 5 amino acids within the sequence of amino acid 1-19 of mature murine ADM (SEQ ID No.: 17).

In a specific embodiment according to the present invention a non-neutralizing anti-ADM antibody or anti-ADM antibody fragment or ADM non-Ig scaffold is used, wherein said anti-ADM antibody or an anti-ADM antibody fragment blocks the bioactivity of ADM to less than 80%, preferably less than 50% (of baseline values). It has to be understood that said limited blocking of the bioactivity (meaning reduction of the bioactivity) of ADM occurs even at excess concentration of the antibody, fragment or scaffold, meaning an excess of the antibody, fragment or scaffold in relation to ADM. Said limited blocking is an intrinsic property of the ADM binder itself in said specific embodiment. This means that said antibody, fragment or scaffold has a maximal inhibition of 80% or 50% respectively. In a preferred embodiment said anti-ADM antibody, anti-ADM antibody fragment or anti-ADM non-Ig scaffold would block the bioactivity/reduce the bioactivity of anti-ADM to at least 5%. The stated above means that approximately 20% or 50% or even 95% residual ADM bioactivity remains present, respectively.

Thus, in accordance with the present invention the provided anti-ADM antibodies, anti-ADM antibody fragments, and anti-ADM non-Ig scaffolds do not neutralize the respective ADM bioactivity.

The bioactivity is defined as the effect that a substance takes on a living organism or tissue or organ or functional unit in vivo or in vitro (e.g. in an assay) after its interaction. In case of ADM bioactivity this may be the effect of ADM in a human recombinant ADM receptor cAMP functional assay. Thus, according to the present invention bioactivity is defined via an ADM receptor cAMP functional assay.

The following steps may be performed in order to determine the bioactivity of ADM in such an assay:
Dose response curves are performed with ADM in said human recombinant ADM receptor cAMP functional assay.
The ADM concentration of half-maximal cAMP stimulation may be calculated.
At constant half-maximal cAMP-stimulating ADM concentrations dose response curves (up to 100 μg/ml final concentration) are performed by an ADM stabilizing antibody or ADM stabilizing antibody fragment or ADM stabilizing non-Ig scaffold, respectively.

A maximal inhibition in said ADM bioassay of 50% means that said anti-ADM antibody or said anti-ADM antibody fragment or said anti-ADM non-Ig scaffold, respectively, blocks the bioactivity of ADM 35 to 50% of baseline values. A maximal inhibition in said ADM bioassay of 80% means that said anti-ADM antibody or said anti-ADM antibody fragment or said anti-ADM non-Ig scaffold, respectively, blocks the bioactivity of ADM to 80%. This is in the sense of blocking the ADM bioactivity to not more than 80%. This means approximately 20% residual ADM bioactivity remains present.

However, by the present specification and in the above context the expression “blocks the bioactivity of ADM” in relation to the herein disclosed anti-ADM antibodies, anti-ADM antibody fragments, and anti-ADM non-Ig scaffolds should be understood as mere decreasing the bioactivity of ADM from 100% to 20% remaining ADM bioactivity at maximum, preferably decreasing the ADM bioactivity from 100% to 50% remaining ADM bioactivity; but in any case there is ADM bioactivity remaining that can be determined as detailed above. The bioactivity of ADM may be determined in a human recombinant ADM receptor cAMP functional assay (Adrenomedullin Bioassay) according to Example 2.

In a preferred embodiment a modulating anti-ADM antibody or a modulating anti-ADM antibody fragment or a modulating anti-ADM non-Ig scaffold is used in therapy or prevention of shock in a patient.

A “modulating” anti-ADM antibody or a modulating anti-ADM antibody fragment or a modulating anti-ADM non-Ig scaffold is an antibody or antibody fragment or non-Ig scaffold that enhances the half-life (t_{1/2} half retention time) of adrenomedullin in serum, blood, plasma at least 10%, preferably at least, 50%, more preferably >50%, most preferably >100% and blocks the bioactivity of ADM to less than 80%, preferably less than 50% and said anti-ADM antibody, anti-ADM antibody fragment or anti-ADM non-Ig scaffold would block the bioactivity of ADM to at least 5%. These values related to half-life and blocking of bioactivity have to be understood in relation to the before-mentioned assays in order to determine these values. This is in the sense of blocking the ADM bioactivity of not more than 80% or not more than 50%, respectively.

Such a modulating anti-ADM antibody or modulating anti-ADM antibody fragment or a modulating anti-ADM non-Ig scaffold offers the advantage that the dosing of the administration is facilitated. The combination of partially blocking or partially reducing ADM bioactivity and increase of the in vivo half-life (increasing the ADM bioactivity) leads to beneficial simplicity of anti-ADM antibody or an anti-ADM antibody fragment or anti-ADM non-Ig scaffold dosing. In a situation of excess of endogenous ADM (maximal stimulation, late sepsis phase, shock, hypodynamic phase) the activity lowering effect is the major impact of the antibody or fragment or scaffold, limiting the (negative) effect of ADM. In case of low or normal endogenous ADM concentrations, the biological effect of anti-ADM antibody or anti-ADM antibody fragment or anti-ADM non-Ig scaffold is a combination of lowering (by partially blocking) and increase by increasing the ADM half-life. Thus, the non-neutralizing and modulating anti-ADM antibody or anti-ADM antibody fragment or anti-ADM non-Ig scaffold acts like an ADM bioactivity buffer in order to keep the bioactivity of ADM within a certain physiological range.

In a specific embodiment of the invention the antibody is a monoclonal antibody or a fragment thereof. In one embodiment of the invention the anti-ADM antibody or the anti-ADM antibody fragment is a human or humanized antibody or derived therefrom. In one specific embodiment one or more (murine) CDR's are grafted into a human antibody or antibody fragment.

Subject matter of the present invention in one aspect is a human or humanized CDR-grafted antibody or antibody fragment thereof that binds to ADM, wherein the human or humanized CDR-grafted antibody or antibody fragment thereof comprises an antibody heavy chain (H chain) comprising:

(SEQ ID No.: 1)
GYTFSRYW,
(SEQ ID No.: 2)
ILPGSGST
and/or
(SEQ ID No.: 3)
TEGYEYDGFDY

and/or further comprises an antibody light chain (L chain) comprising:

(SEQ ID No.: 4)
QSIVYSNGNTY,
RVS (not part of the Sequencing Listing)
and/or
(SEQ ID No.: 5)
FQGSHIPYT.

In one specific embodiment of the invention subject matter of the present invention is a human or humanized monoclonal antibody that binds to ADM or an antibody fragment thereof that binds to ADM wherein the heavy chain comprises at least one CDR selected from the group comprising:

(SEQ ID No.: 1)
GYTFSRYW,
(SEQ ID No.: 2)
ILPGSGST,
(SEQ ID No.: 3)
TEGYEYDGFDY

and wherein the light chain comprises at least one CDR selected from the group comprising:

(SEQ ID No.: 4)
QSIVYSNGNTY,
RVS (not part of the Sequencing Listing),
(SEQ ID No.: 5)
FQGSHIPYT.

In a more specific embodiment of the invention subject matter of the invention is a human monoclonal antibody that binds to ADM or an antibody fragment thereof that binds to ADM wherein the heavy chain comprises the sequences:

(SEQ ID No.: 1)
GYTFSRYW,
(SEQ ID No.: 2)
ILPGSGST,
(SEQ ID No.: 3)
TEGYEYDGFDY

and wherein the light chain comprises the sequences:

(SEQ ID No.: 4)
QSIVYSNGNTY,
RVS (not part of the Sequencing Listing),
(SEQ ID No.: 5)
FQGSHIPYT.

In a very specific embodiment, the anti-ADM antibody has a sequence selected from the group comprising: SEQ ID No. 6, 7, 8, 9, 10, 11, 12, 13, 32 and 33.

The anti-ADM antibody or anti-ADM antibody fragment or anti-ADM non-Ig scaffold according to the present invention exhibits an affinity towards human ADM in such that affinity constant is greater than 10⁻⁷ M, preferred 10⁻⁸ M, preferred affinity is greater than 10⁻⁹ M, most preferred higher than 10⁻¹⁰ M. A person skilled in the art knows that it may be considered to compensate lower affinity by applying a higher dose of compounds and this measure would not lead out-of-the-scope of the invention. The affinity constants may be determined according to the method as described in Example 1.

Subject matter of the present invention is a human or humanized monoclonal antibody or fragment that binds to ADM or an antibody fragment thereof for use in therapy or prevention of shock in a patient according to the present invention, wherein said antibody or fragment comprises a sequence selected from the group comprising:

SEQ ID NO: 6
(AM-VH-C)
SEQ ID NO: 7
(AM-VH1)
SEQ ID NO: 8
(AM-VH2-E40)
SEQ ID NO: 9
(AM-VH3-T26-E55)
SEQ ID NO: 10
(AM-VH4-T26-E40-E55)
SEQ ID NO: 11
(AM-VL-C)
SEQ ID NO: 12
(AM-VL1)
SEQ ID NO: 13
(AM-VL2-E40)

Another embodiment of the invention relates to a human or humanized monoclonal antibody or fragment that binds to ADM or an antibody fragment thereof for use in therapy or prevention of shock in a patient, wherein said antibody or fragment comprises the following sequence as a heavy chain:

SEQ ID NO: 32

[symbol]

[symbol]

indicates data missing or illegible when filed

and comprises the following sequence as a light chain:

SEQ ID NO: 33

[symbol]

[symbol]

indicates data missing or illegible when filed

In a specific embodiment of the invention the antibody comprises the following sequence as a heavy chain:

SEQ ID NO: 32

[symbol]

[symbol]

indicates data missing or illegible when filed

or a sequence that is >95% identical to it, preferably >98%, preferably >99% and comprises the following sequence as a light chain:

SEQ ID NO: 33

[symbol]

[symbol]

indicates data missing or illegible when filed

or a sequence that is >95% identical to it, preferably >98%, preferably >99%, wherein the heavy chain comprises the sequences:

CDR1:
SEQ ID NO: 1
GYTFSRYW
CDR2:
SEQ ID NO: 2
ILPGSGST
CDR3:
SEQ ID NO: 3
TEGYEYDGFDY

and wherein the light chain comprises the sequences:

CDR1:
SEQ ID NO: 4
QSIVYSNGNTY
CDR2:
RVS
CDR3:
SEQ ID NO: 5
FQGSHIPYT.

This means, in one embodiment of the invention the CDR's do not exhibit any variations of the sequence. Any variation of the above sequence is outside of the CDR's in said embodiment.

To assess the identity between two amino acid sequences, a pairwise alignment is performed. Identity defines the percentage of amino acids with a direct match in the alignment.

An epitope, also known as antigenic determinant, is the part of an antigen that is recognized by the immune system, specifically by antibodies. For example, the epitope is the specific piece of the antigen to which an antibody binds. The part of an antibody that binds to the epitope is called a paratope. The epitopes of protein antigens are divided into two categories, conformational epitopes and linear epitopes, based on their structure and interaction with the paratope.

Conformational and linear epitopes interact with the paratope based on the 3-D conformation adopted by the epitope, which is determined by the surface features of the involved epitope residues and the shape or tertiary structure of other segments of the antigen. A conformational epitope is formed by the 3-D conformation adopted by the interaction of discontiguous amino acid residues. A linear or a sequential epitope is an epitope that is recognized by antibodies by its linear sequence of amino acids, or primary structure and is formed by the 3-D conformation adopted by the interaction of contiguous amino acid residues.

One embodiment of the invention relates to a method for the prediction of an increase of DPP3 in a critically ill patient, wherein a patient is selected for therapy/treatment with DPP3 inhibitors if the level of DPP3 in said sample is above said pre-determined threshold.

One embodiment of the invention relates to a method for the prevention of a DPP3 increase in a critical ill patient, wherein a DPP3 inhibitor is administered if the level of DPP3 in said sample is above a predetermined threshold.

One embodiment of the invention relates to a DPP3 inhibitor for use in the prevention of a DPP3 increase in a critical ill patient, wherein said patient has a level of DPP3 above a threshold.

In another specific embodiment of the invention said DPP3 inhibitor is an anti-DPP3-antibody or anti-DPP3-antibody fragment or anti-DPP3 scaffold.

In another specific embodiment of the invention said inhibitor is an anti-DPP3-antibody or anti-DPP3-antibody fragment or or anti-DPP3 scaffold that binds to a region or epitope of DPP3 of at least 4 to 5 amino acids in length comprised in SEQ ID No. 36.

In another specific embodiment of the invention said inhibitor is an anti-DPP3 antibody or anti-DPP3 antibody fragment or anti-DPP3 scaffold that binds to a region or epitope of DPP3 of at least 4 to 5 amino acids in length comprised in SEQ ID No. 37.

In another preferred embodiment of the present invention said inhibitor is an anti-DPP3 antibody or anti-DPP3 antibody fragment or anti-DPP3 scaffold that binds to a region or epitope of DPP3 that is located in SEQ ID No. 38.

In another preferred embodiment of the present invention said anti-DPP3 antibody or anti-DPP3 antibody fragment or anti-DPP3 scaffold that binds to a region or epitope of DPP3 that is located in SEQ ID No. 39.

In another specific embodiment of the invention said inhibitor is an anti-DPP3 antibody or anti-DPP3 antibody fragment or anti-DPP3 scaffold that exhibits a minimum binding affinity to DPP3 of equal or less than 10⁻⁷ M.

In another specific embodiment of the invention said inhibitor is an anti-DPP3 antibody or anti-DPP3 antibody fragment or anti-DPP3 scaffold and inhibits activity of DPP3 of at least 10%, or at least 50%, more preferred at least 60%, even more preferred more than 70%, even more preferred more than 80%, even more preferred more than 90%, even more preferred more than 95%.

In another specific embodiment of the invention said antibody is a monoclonal antibody or monoclonal antibody fragment.

In another specific embodiment of the invention said inhibitor is a monoclonal anti-DPP3 antibody or anti-DPP3 antibody fragment, wherein the complementarity determining regions (CDR's) in the heavy chain comprises the sequences:
SEQ ID NO.: 42, SEQ ID NO.: 43 and/or SEQ ID NO.: 44
and the complementarity determining regions (CDR's) in the light chain comprises the sequences:
SEQ ID NO.: 45, KVS and/or SEQ ID NO.: 46.

In another specific embodiment of the invention said inhibitor is a monoclonal anti-DPP3 antibody or anti-DPP3 antibody fragment, wherein said monoclonal antibody or antibody fragment is a humanized monoclonal antibody or humanized monoclonal antibody fragment.

In another specific embodiment of the invention said inhibitor is a humanized monoclonal anti-DPP3 antibody or anti-DPP3 antibody fragment, wherein the heavy chain comprises the sequence:
SEQ ID NO.: 40
and wherein the light chain comprises the sequence:
SEQ ID NO.: 41.

In another specific embodiment of the invention said inhibitor is a humanized monoclonal anti-DPP3 antibody or anti-DPP3 antibody fragment, with the heavy chain consisting of the sequence:
SEQ ID NO.: 47
and with the light chain consisting of the sequence:
SEQ ID NO.: 48.

Procizumab, a humanized monoclonal IgG1 antibody with a heavy chain consisting of the sequence SEQ ID NO.: 47 and with a light chain consisting of the sequence SEQ ID NO.: 48, specifically binds circulating DPP3, targets and modulates DPP3 activity, an essential regulator of cardiovascular function. Its mode of action is relevant in acute diseases that are associated with massive cell death and uncontrolled release of intracellular DPP3 into the bloodstream. Translocated DPP3 remains active in the circulation where it cleaves bioactive peptides in an uncontrolled manner. Procizumab is able to block circulating DPP3, inhibiting bioactive peptide degradation in the bloodstream. This blockade results in stabilization of cardiovascular and renal function and reduction of short-term mortality. As shown in the Example part, preclinical studies of Procizumab in animal models of cardiovascular failure showed impressive and instant efficacy. As an example, injection of Procizumab in rats with shock-induced cardiovascular failure led to an instant normalization of shortening. In several preclinical cardiovascular failure models, Procizumab has shown to improve all clinically relevant endpoints in vivo. It normalizes ejection fraction and kidney function and reduces mortality.

Throughout the specification the “antibodies”, or “antibody fragments”, or “scaffolds” in accordance with the invention are capable to bind DPP3, and thus are directed against DPP3, and thus can be referred to as “anti-DPP3 antibodies”, “anti-DPP3 antibody fragments or “anti-DPP3 scaffolds”.

The term “antibody” generally comprises monoclonal and polyclonal antibodies and binding fragments thereof, in particular Fc-fragments as well as so called “single-chain-antibodies” (Bird et al. 1988), chimeric, humanized, in particular CDR-grafted antibodies, and dia or tetrabodies (Holliger et al. 1993). Also comprised are immunoglobulin-like proteins that are selected through techniques including, for example, phage display to specifically bind to the molecule of interest contained in a sample. In this context the term “specific binding” refers to antibodies raised against the molecule of interest or a fragment thereof. An antibody is considered to be specific, if its affinity towards the molecule of interest or the aforementioned fragment thereof is at least preferably 50-fold higher, more preferably 100-fold higher, most preferably at least 1000-fold higher than towards other molecules comprised in a sample containing the molecule of interest. It is well known in the art how to make antibodies and to select antibodies with a given specificity.

In one embodiment of the invention the anti-DPP3 antibody or anti-DPP3 antibody fragment or anti-DPP3 non-Ig scaffold is monospecific.

Monospecific anti-DPP3 antibody or monospecific anti-DPP3 antibody fragment or monospecific anti-DPP3 non-Ig scaffold means that said antibody or antibody fragment or non-Ig scaffold binds to one specific region encompassing at least 5 amino acids within the target DPP3 (SEQ ID No. 36). Monospecific anti-DPP3 antibody or monospecific anti-DPP3 antibody fragment or monospecific anti-DPP3 non-Ig scaffold are anti-DPP3 antibodies or anti-DPP3 antibody fragments or anti-DPP3 non-Ig scaffolds that all have affinity for the same antigen. Monoclonal antibodies are monospecific, but monospecific antibodies may also be produced by other means than producing them from a common germ cell.

An antibody or fragment according to the present invention is a protein including one or more polypeptides substantially encoded by immunoglobulin genes that specifically binds an antigen. The recognized immunoglobulin genes include the kappa, lambda, alpha (IgA), gamma (IgG1, IgG2, IgG3, IgG4), delta (IgD), epsilon (IgE) and mu (IgM) constant region genes, as well as the myriad immunoglobulin variable region genes. Full-length immunoglobulin light chains are generally about 25 Kd or 214 amino acids in length.

Full-length immunoglobulin heavy chains are generally about 50 Kd or 446 amino acid in length. Light chains are encoded by a variable region gene at the NH2-terminus (about 110 amino acids in length) and a kappa or lambda constant region gene at the COOH-terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)2, as well as bifunctional hybrid antibodies and single chains (e.g., Lanzavecchia et al. 1987. Eur. J. Immunol. 17: 105; Huston et al. 1988. Proc. Natl. Acad. Sci. U.S.A., 85: 5879-5883; Bird et al. 1988. Science 242: 423-426; Hood et al. 1984, Immunology, Benjamin, NY., 2nd ed.; Hunkapiller and Hood 1986. Nature 323:15-16). An immunoglobulin light or heavy chain variable region includes a framework region interrupted by three hypervariable regions, also called complementarity determining regions (CDR's) (see, Sequences of Proteins of Immunological Interest, E. Kabat et al. 1983, US. Department of Health and Human Services). As noted above, the CDRs are primarily responsible for binding to an epitope of an antigen. An immune complex is an antibody, such as a monoclonal antibody, chimeric antibody, humanized antibody or human antibody, or functional antibody fragment, specifically bound to the antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody, although other mammalian species can be used, or the variable region can be produced by molecular techniques. Methods of making chimeric antibodies are well known in the art, e.g., see U.S. Pat. No. 5,807,715. A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDR's. The acceptor framework of a humanized immunoglobulin or antibody may have a limited number of substitutions by amino acids taken from the donor framework. Humanized or other monoclonal antibodies can have additional conservative amino acid substitutions, which have substantially no effect on antigen binding or other immunoglobulin functions. Exemplary conservative substitutions are those such as gly, ala; val, ile, leu; asp, glu; asn, gln; ser, thr; lys, arg; and phe, tyr. Humanized immunoglobulins can be constructed by means of genetic engineering (e.g., see U.S. Pat. No. 5,585,089). A human antibody is an antibody wherein the light and heavy chain genes are of human origin. Human antibodies can be generated using methods known in the art. Human antibodies can be produced by immortalizing a human B cell secreting the antibody of interest. Immortalization can be accomplished, for example, by EBV infection or by fusing a human B cell with a myeloma or hybridoma cell to produce a trioma cell. Human antibodies can also be produced by phage display methods (see, e.g., WO91/17271; WO92/001047; WO92/20791), or selected from a human combinatorial monoclonal antibody library (see the Morphosys website). Human antibodies can also be prepared by using transgenic animals carrying a human immunoglobulin gene (for example, see WO93/12227; WO 91/10741).

Thus, the anti-DPP3 antibody may have the formats known in the art. Examples are human antibodies, monoclonal antibodies, humanized antibodies, chimeric antibodies, CDR-grafted antibodies. In a preferred embodiment antibodies according to the present invention are recombinantly produced antibodies as e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH3 domain; bivalent Fab or multivalent Fab, e.g. formed via multimerization with the aid of a heterologous domain, e.g. via dimerization of dHLX domains, e.g. Fab-dHLX-FSx2; F(ab′)2-fragments, scFv-fragments, multimerized multivalent or/and multi-specific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulines and numerous others.

In a preferred embodiment the anti-DPP3 antibody format is selected from the group comprising Fv fragment, scFv fragment, Fab fragment, scFab fragment, F(ab)2 fragment and scFv-Fc Fusion protein. In another preferred embodiment the antibody format is selected from the group comprising scFab fragment, Fab fragment, scFv fragment and bioavailability optimized conjugates thereof, such as PEGylated fragments. One of the most preferred formats is the scFab format.

In one embodiment of the invention anti-DPP3 antibodies according to the present invention may be produced as outlined in Example 1 by synthesizing fragments of DPP3 as antigens or full-length DPP3. Thereafter, binder to said fragments are identified using the below described methods or other methods as known in the art.

Humanization of murine antibodies may be conducted according to the following procedure: For humanization of an antibody of murine origin the antibody sequence is analyzed for the structural interaction of framework regions (FR) with the complementary determining regions (CDR) and the antigen. Based on structural modelling an appropriate FR of human origin is selected and the murine CDR sequences are transplanted into the human FR. Variations in the amino acid sequence of the CDRs or FRs may be introduced to regain structural interactions, which were abolished by the species switch for the FR sequences. This recovery of structural interactions may be achieved by random approach using phage display libraries or via directed approach guided by molecular modelling (Almagro and Fransson 2008. Humanization of antibodies. Front Biosci. 13:1619-33).

### Methods for Obtaining Monoclonal Antibodies

In all of the following embodiments, the term monoclonal antibody is meant to include monoclonal antibodies, as well as fragments of monoclonal antibodies, such as the ones detailed herein, more particularly monoclonal antibodies.

### Hybridoma

In a further aspect, the antibody according to the present invention is a monoclonal antibody obtainable by a method comprising:
i) fusing antibody-secreting cells from an animal previously immunized with an antigen with myeloma cells to obtain a multitude of hybridomas,
ii) isolating from said multitude of hybridomas a hybridoma producing a desired monoclonal antibody.

In certain embodiments, the antibody according to the present invention is a monoclonal antibody obtainable by isolating from a multitude of hybridomas a hybridoma producing a desired monoclonal antibo, wherein said multitude of hybridomas were produced by fusing antibody-secreting cells from an animal previously immunized with an antigen with myeloma cells to obtain multitude of hybridomas.

A desired monoclonal antibody is in particular a monoclonal antibody binding the antigen, in particular with a binding affinity of at least 10⁷ M⁻¹, preferred 10⁸ M⁻¹, more preferred affinity is greater than 10⁹ M⁻¹, most preferred greater than 10¹⁰ M⁻¹.

In certain embodiments of the method for obtaining an antibody, in step i) the animal is a mammal, particularly a rabbit, a mouse or a rat, more particularly a mouse, more particularly a Balb/c mouse.

In certain embodiments of the method for obtaining an antibody, in step i) the antibody-secreting cell is a splenocyte, more particularly an activated B-cell.

In certain embodiments of the method for obtaining an antibody, in step i) fusing involves the use of polyethylene glycol.

In certain embodiments of the method for obtaining an antibody, in step i) the myeloma is derived from a mammal, in certain embodiments from the same species of mammal from which the multitude of antibody-secreting cells is obtained. In certain specific embodiments of the method for obtaining an antibody, in step i) the myeloma cells are of the cell line SP2/0.

In certain embodiments of the method for obtaining an antibody, said fusing in step i) comprises PEG-assisted fusion, Sendai virus-assisted fusion or electric current-assisted fusion.

In certain embodiments of the method for obtaining an antibody, said isolating in step ii) comprises performing an antibody capture assay, an antigen capture assay, and/or a functional screen.

In certain embodiments of the method for obtaining an antibody, in step ii) isolating the hybridoma producing a desired monoclonal antibody may involve cloning and re-cloning the hybridomas using the limiting-dilution technique.

In one embodiment, said antibody capture assay comprises
a) binding an antigen to a substrate, particularly a solid substrate,
b) allowing the produced antibodies to bind to the antigen,
c) removing unbound antibodies by washing,
d) detecting bound antibodies.

In one embodiment, said antigen capture assay comprises
a) binding the produced antibodies to a substrate, particularly a solid substrate, b) allowing antigen to bind to said antibodies,
c) removing unbound antigen by washing,
d) detecting bound antigen;
or said antigen capture assay comprises
a) allowing an antigen to bind the produced antibodies to form an antibody-antigen complex,
b) binding said antibody-antigen complex to a substrate, particularly a solid substrate,
c) removing unbound antigen by washing,
d) detecting bound antigen.

In one embodiment, said isolating of step ii) comprises performing an enzyme-linked immunosorbent assay, fluorescence-activated cell sorting, cell staining, immunoprecipitation, and/or a western blot.

In one embodiment, said detecting of the antibody or the antigen is accomplished with an immunoassay.

In one embodiment, the animal is a transgenic animal, in particular a transgenic mouse (wherein in particular the mouse immunoglobulin (Ig) gene loci have been replaced with human loci within the transgenic animal genome), such as HuMabMouse or XenoMouse.

In one embodiment, the antigen comprises a peptide as described herein in Table 1, or Table 6 respectively, which in certain embodiments (in particular for immunization) may be conjugated to a protein, particularly a serum protein, more particularly a serum albumin, more particularly BSA.

In a preferred embodiment, the antibody according to the present invention is a monoclonal antibody obtainable by a method comprising:
i) fusing splenocytes cells from a Balb/c mouse previously immunized with a peptide as described herein in Table 1 or 6 with SP2/0 myeloma cells using polyethylene glycol, to obtain a multitude of hybridomas,
ii) isolating from said multitude of hybridomas a hybridoma producing a desired monoclonal antibody;
   more preferably, the method comprises
growing hybridomas for a first period (in particular 2 weeks) in HAT medium [RPMI 1640 culture medium supplemented with 20% fetal calf serum and HAT-Supplement]
followed replacing HAT medium with HT Medium for a multitude of passages (in particular 3)
followed by returning to the normal cell culture medium for a second time period, in particular until the end of three weeks after fusion
primary screening of Cell culture supernatants for antigen-specific IgG antibodies
propagating microcultures of cells that tested positive in 4)
retesting Cell culture supernatants of microcultures for antigen-specific IgG antibodies
cloning and re-cloning cultures that tested positive in 6), using the limiting-dilution technique
optionally determining the isotypes of clones obtained from 7)
optionally purifying antibodies via Protein A

### Phage Display

In a further aspect, the antibody according to the present invention is a monoclonal antibody obtainable by a method comprising:
i) isolating at least one antibody having affinity to an antigen from an antibody gene library;
ii) generating at least one cell strain expressing said at least one antibody;
iii) isolating the at least one antibody from a culture of the at least one cell strain obtained in step ii).

An antibody having affinity to an antigen is in particular an antibody with a binding affinity of at least 10⁷ M⁻¹, preferred 10⁸ M⁻¹, more preferred affinity is greater than 10⁹ M⁻¹, most preferred greater than 10¹⁰ M⁻¹.

In a certain embodiment, the antibody according to the present invention is a monoclonal antibody obtainable by isolating at least one antibody from a culture derived from at least one cell strain which expressed at least one antibody having affinity to an antigen from an antibody gene library.

In one embodiment, the antigen comprises a peptide as described herein in Table 1, or Table 6 respectively, which in certain embodiments may be bound to a solid phase.

In certain embodiments of the method for obtaining an antibody, in step i) the antibody gene library is a naive antibody gene library, particularly a human naive antibody gene library, more particularly in said library the antibodies are presented via phage display, i.e. on phages comprising a nucleotide sequence encoding for such respective antibody; more particularly the library HAL 7, HAL 8, or HAL 9, more particularly a library comprising the human naive antibody gene libraries HAL7/8.

In certain embodiments of the method for obtaining an antibody, in step i) screening comprises the use of an antigen, particularly an antigen containing a tag, more particularly a biotin tag, linked thereto via two different spacers. In particular embodiments, such panning strategy includes a mix of panning rounds with non-specifically bound antigen and antigen bound specifically via the tag, in the case of a biotin tag, bound to streptavidin. In this way, the background of non-specific binders may be minimized.

In certain embodiments of the method for obtaining an antibody, in step i), in embodiments wherein the library is a phage display library, the antibody is isolated by isolating a phage presenting said antibody (and comprising a nucleotide sequence encoding for the antibody).

In certain embodiments of the method for obtaining an antibody, in step ii) said cell strain is generated via introduction of a nucleotide sequence encoding for the antibody;), in embodiments wherein the library in step i) is a phage display library, the isolated phage from step i) may be used to produce a bacterial strain, e.g. an E. coli strain, expressing the antibody.

In certain embodiments of the method for obtaining an antibody, in step iv); in embodiments wherein the library in step i) is a phage display library and wherein a bacterial strain is produced in step ii), antibody may be isolated from the supernatant of the culture.

It is understood that, as used in describing the methods for obtaining an antibody, the term “one antibody” in the expression “at least one antibody” in particular may include more than one antibody molecule of antibodies having the same amino acid sequence. This understanding applies, mutatis mutandis, to the term “one cell strain”.

In certain embodiments of the method for obtaining an antibody, more than one antibody (referring to a multitude of antibodies having distinct amino acid sequences, respectively) is isolated in step i) and accordingly more than one cell strain is generated in step ii). Such method may involve the selection of clones that are positive for binding to the antigen, e.g. via a binding assay, e.g. an ELISA assay involving the antigen, and cells positive for binding to the antigen may be isolated to produce monoclonal cell strains.

In a preferred embodiment, the antibody according to the present invention is a monoclonal antibody obtainable by a method comprising:
i) isolating at least one antibody having affinity to an antigen from an antibody gene library comprising the human naive antibody gene libraries HAL7/8, by eluting phages carrying said antibody from the library;
ii) generating at least one E. coli cell strain expressing said at least one antibody;
iii) isolating the at least one antibody from the supernantant a culture of the at least one E. coli cell strain obtained in step ii).

In a further aspect, an antibody fragment according to the present invention is produced by a method in volving enzymatic digestion of an antibody. In certain embodiments, this method produces e.g. Fab or F(ab)2 antibody fragments. In certain embodiments, this method involves digestion with pepsin or papain, which are optionally immobilized on a surface.

In certain embodiments, antibodies may be humanized by CDR-grafting, in particular by a process involving the steps:
extracting RNA from hybridomas expressing an antibody of interest (e.g. obtained by a method as described herein);
amplifying said extracted RNA via RT-PCR, in particular with primer sets specific for the heavy and light chains of the antibody of interest, to obtain to obtain a DNA product;
further amplifying said DNA product via PCR, in particular using semi-nested primer sets specific for antibody variable regions;
determining the sequence of the DNA product;
aligning said sequence with homologous human framework sequences to determine a humanized sequence for the variable heavy chain and the variable light chain sequences (of the desired antibody).

In certain embodiments, antibodies may be humanized by aligning the sequence of a DNA product that was obtained by amplifying RNA extracted from hybridomas expressing an antibody of interest via RT-PCR, in particular with primer sets specific for the heavy and light chains of the antibody of interest and further amplifying the DNA obtained therefrom via PCR, in particular using semi-nested primer sets specific for antibody variable regions, with homologous human framework sequences to determine a humanized sequence for the variable heavy chain and the variable light chain sequences (of the desired antibody).

In certain embodiments, antibodies may be humanized by
determining the complementary determining regions (CDR), which may be accomplished by analyzing the structural interaction of framework regions (FR) with the complementary determining regions (CDR) and the antigen;
transplanting said CDR sequences into a human framework region.

In certain embodiments, antibodies may be humanized by transplanting CDR sequences, which may preferably have been determined by analyzing the structural interaction of framework regions (FR) with the complementary determining regions (CDR) and the antigen, into a human framework region.

In certain embodiments variations in the amino acid sequence of the CDRs or FRs may be introduced to maintain structural interactions with the antigen (which may otherwise be abolished by introducing the human FR sequences), for instance by a random approach using phage display libraries or via directed approach guided by molecular modeling.

The DNA sequences encoding for antibodies determined as detailed herein can be transferred by known genetic engineering techniques into cells and used for production of the antibody.

### Producing Antibodies

In a further aspect, the antibody according to the present invention is a monoclonal antibody obtainable by the methods described herein, produced by a method comprising:
culturing a cell strain comprising a nucleotide sequence encoding for the antibody;
isolating the antibody from said culture.

In a further certain aspect, the antibody according to the present invention is a monoclonal antibody obtainable by the methods described herein, produced by isolating the antibody from a culture of a cell strain comprising a nucleotide sequence encoding for said antibody.

In certain embodiments of said method, the cell strain is produced as described herein above and may comprise bacterial cells, such as gram-negative bacteria, e.g. E. coli, Proteus mirabilis, or Pseudomonas putidas, gram-positive bacteria, e.g. Bacillus brevis, Bacillus subtilis, Bacillus megaterium, Lactobacilli such as Lactobacillus zeae/casei or Lactobacillus paracasei, or Streptomyces, such as Streptomyces lividans; eucariotic cells such as yest, e.g. Pichia pastoris, Saccharomyces cerevisiae, Hansenula polymorpha, Schizosaccharomyces pombe, Schwanniomyces occidentalis, Kluyveromyces lactis, or Yarrowia lipolytica; fugi, such as filamentous fungi, e.g. of the genus Trichoderma of Aspergillus, such as A. niger (e.g. subgenus A. awamori) and Aspergillus oryzae, Trichoderma reesei, Chrysosporium, such as C. lucknowense; protozoae, such as Leishmania, e.g. L. tarentolae; insect cells, such as insect cells transfected a Baculovirus, e.g. AcNPV, such as insect cell lines from Spodoptera frugiperda, e.g. Sf-9 or Sf-21, Drosophila melanogaster, e.g. DS2, or Trichopulsia ni, e.g. High Five cells (BTI-TN-5B1-4); mammalian cells such as hamster, e.g. Chinese hamster ovary such as K1-, DukX B11-, DG44, Lec13, or BHK, mouse, e.g. mouse myeloma such as NS0, Homo sapiens, e.g. Per.C6, AGE1.HN, HEK293.

In certain embodiments of said method, the cells may be hybridoma cells, e.g. as described herein.

In certain embodiments of said method, culturing may take place in a static suspension culture, an agitated suspension culture, a membrane-based culture, a matrix-based culture or a high cell density bioreactor; a vessel for such culturing may be selected from the group comprising a T-flask, a roller culture, a spinner culture, a stirred tank bioreactor, an airlift bioreactor, a static membrane-based or matrix-based culture system, a suspension bioreactor, a fluidized bed bioreactor, a ceramic bioreactor, a perfusion system, a hollow fiber bioreactor.

In certain embodiments of said method, the cells may be immobilized on a matrix.

A high cell density bioreactor is in particular a culture system capable of generating cell densities greater than 10\hat{ }8 cells/ml.

In a further aspect, the antibody according to the present invention is a monoclonal antibody obtainable by the methods described herein, produced by a method comprising:
generating a transgenic plant or animal comprising a nucleotide sequence encoding for the antibody;
isolating the antibody from said plant or animal or a secretion or product of said plant or animal.

In a certain further aspect, the antibody according to the present invention is a monoclonal antibody obtainable by the methods described herein, produced by isolating the antibody from a transgenic plant or transgenic animal or a secretion or product of a transgenic plant or transgenic animal having a nucleotide sequence encoding for the antibody.

Said animal may e.g. be selected from a chicken, a mouse, a rat, a rabbit, a cow, a goat, a sheep, a pig; said secretion or product may e.g. be milk or an egg. Said plant may e.g. be selected from tobacco (N. tabacum or N. benthamiana), duckweed (Lemna minor), Chlamydomonas reinhardtii, rice, Arabidopsis thaliana, alfalfa (Medicago sativa), lettuce, maize.

The antibodies can in certain embodiments be isolated by physicochemical fractionation, e.g. size exclusion chromatography, precipitation, e.g. using ammonium sulphate, ion exchange chromatography, immobilized metal chelate chromatography gel filtration, zone electrophoresis; based on their classification e.g. binding to bacterial proteins A, G, or L, jacalin; antigen-specific affinity purification via immobilized ligands/antigens; if necessary, low molecular weight components can be removed by methods like dialysis, desalting, and diafiltration.

In some embodiments the antibody is encoded by a nucleotide sequence where the nucleotide sequence is a reverse transcription of an amino acid sequence from an antibody produced by one of the processes described herein.

### FIGURE DESCRIPTION

FIG. 1a: Illustration of antibody formats—Fv and scFv-Variants.

FIG. 1b: Illustration of antibody formats—heterologous fusions and bifunctional antibodies.

FIG. 1c: Illustration of antibody formats—bivalental antibodies and bispecific antibodies.

FIG. 2a: Dose response curve of human ADM. Maximal cAMP stimulation was adjusted to 100% activation.

FIG. 2b: Dose/inhibition curve of human ADM 22-52 (ADM-receptor antagonist) in the presence of 5.63 nM hADM.

FIG. 2c: Dose/inhibition curve of CT-H in the presence of 5.63 nM hADM.

FIG. 2d: Dose/inhibition curve of MR-H in the presence of 5.63 nM hADM.

FIG. 2e: Dose/inhibition curve of NT-H in the presence of 5.63 nM hADM.

FIG. 2f: Dose response curve of mouse ADM. Maximal cAMP stimulation was adjusted to 100% activation.

FIG. 2g: Dose/inhibition curve of human ADM 22-52 (ADM-receptor antagonist) in the presence of 0.67 nM mADM.

FIG. 2h: Dose/inhibition curve of CT-M in the presence of 0.67 nM mADM.

FIG. 2i: Dose/inhibition curve of MR-M in the presence of 0.67 nM mADM.

FIG. 2j: Dose/inhibition curve of NT-M in the presence of 0.67 nM mADM.

FIG. 2k: Shows the inhibition of ADM by F(ab)2 NT-M and by Fab NT-M.

FIG. 2l: shows the inhibition of ADM by F(ab)2 NT-M and by Fab NT-M.

FIG. 3: This figure shows a typical hADM dose/signal curve. And an hADM dose signal curve in the presence of 100 μg/mL antibody NT-H.

FIG. 4: This figure shows the stability of hADM in human plasma (citrate) in absence and in the presence of NT-H antibody.

FIG. 5: Alignment of the Fab with homologous human framework sequences.

FIG. 6: ADM-concentration in healthy human subjects after NT-H application at different doses up to 60 days.

FIG. 7: Inhibition curve of native DPP3 from blood cells with inhibitory antibody AK1967. Inhibition of DPP3 by a specific antibody is concentration dependent, with an at −15 ng/ml when analyzed against 15 ng/ml DPP3.

FIG. 8: Association and dissociation curve of the AK1967-DPP3 binding analysis using Octet. AK1967 loaded biosensors were dipped into a dilution series of recombinant GST-tagged human DPP3 (100, 33.3, 11.1, 3.7 nM) and association and dissociation monitored.

FIG. 9: Western Blot of dilutions of blood cell lysate and detection of DPP3 with AK1967 as primary antibody.

FIG. 10: Procizumab drastically improves shortening fraction (A) and mortality rate (B) in sepsis-induced heart failure rats.

FIG. 11: Experimental design—Isoproterenol-induced cardiac stress in mice followed by Procizumab treatment (B) and control (A).

FIG. 12: Procizumab improved shortening fraction (A) and reduced the renal resistive index (B) within 1 hour and 6 hours after administration, respectively, in isoproterenol-induced heart failure mice.

FIG. 13: High concentrations of DPP3 levels 24 hours after admission of septic patients were associated with worst SOFA scores.

FIG. 14: High DPP3 plasma levels correlate with organ dysfunction in septic patients. Barplots of SOFA score in AdrenOSS-1 according to the evolution of DPP3 levels during ICU stay. HH: DPP3 above median on admission and at 24 h; HL: above median on admission but below median at 24 h; LL: below median on admission and at 24 h; LH: below median on admission but above median at 24 h.

FIG. 15: High concentrations of DPP3 levels 24 hours after admission of septic patients were associated with worst SOFA scores by organ. (A) cardiac, (B) renal, (C) respiratory, (D) liver, (E) coagulation and (F) central nervous system SOFA scores values according to dynamics levels of DPP3 between admission and 24 h (HH: High/High, HL: High/Low, LH: Low/High, LL: Low/Low).

FIG. 16: Kaplan-Meier survival plots in relation to low (<40.5 ng/mL) and high (≥40.5 ng/mL) DPP3 concentrations. (A) 7-day survival of patients with sepsis in relation to DPP3 plasma concentration; (B) 7-day survival of patients with cardiogenic shock in relation to DPP3 plasma concentrations; (C) 7-day survival of patients with septic shock in relation to DPP3 plasma concentration.

FIG. 17: Kaplan-Meier survival plot for all patients (14-day mortality of patients treated with placebo (Plac) or the N-terminal ADM antibody Adrecizumab (Adz)

FIG. 18: Kaplan-Meier survival plot for patients with DPP3<50 ng/mL (14-day mortality of patients treated with placebo (Plac) or the N-terminal ADM antibody Adrecizumab (Adz)

FIG. 19: Kaplan-Meier survival plot for patients with DPP3>50 ng/mL (14-day mortality of patients treated with placebo (Plac) or the N-terminal ADM antibody Adrecizumab (Adz)

FIG. 20: Efficacy of Adrecizumab treatment using different DPP3 threshold values: Kaplan-Meier survival plot (28-day mortality) for patients treated with placebo or the N-terminal ADM antibody Adrecizumab with inclusion of patients (A) with DPP3<50 ng/mL, (B) with DPP3<40 ng/ml, (C) with DPP3<30 ng/ml and (D) with DPP3<22 ng/ml, respectively.

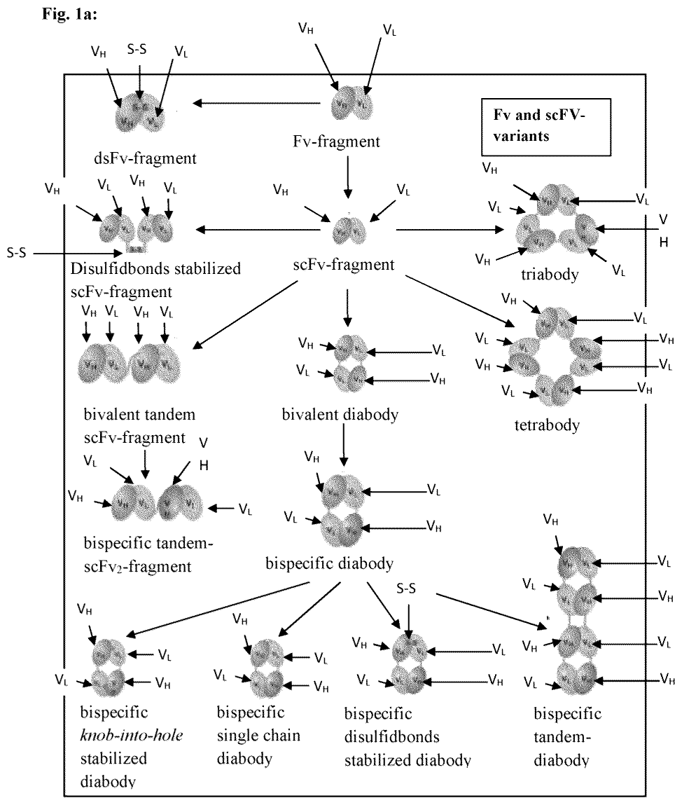

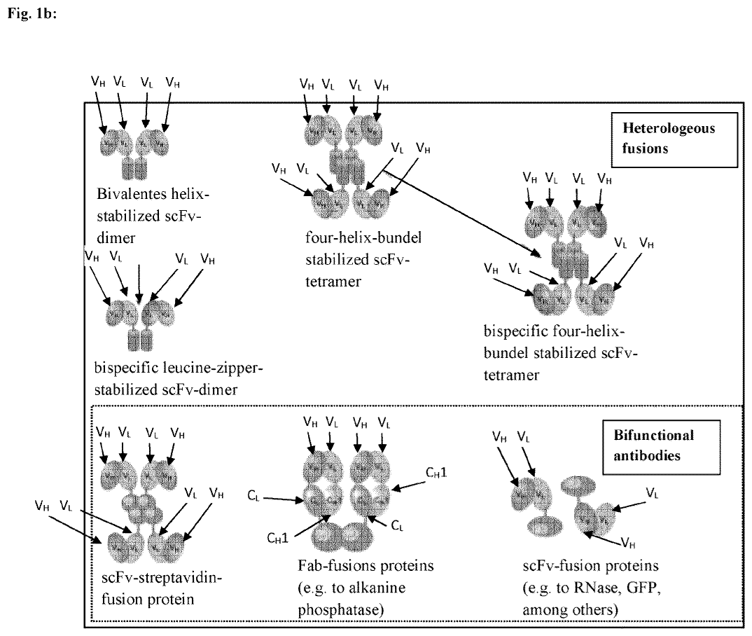

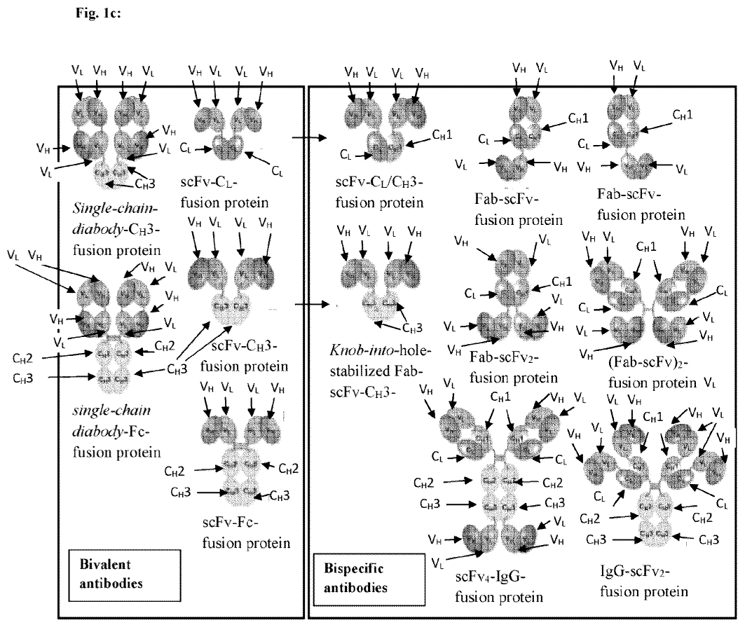

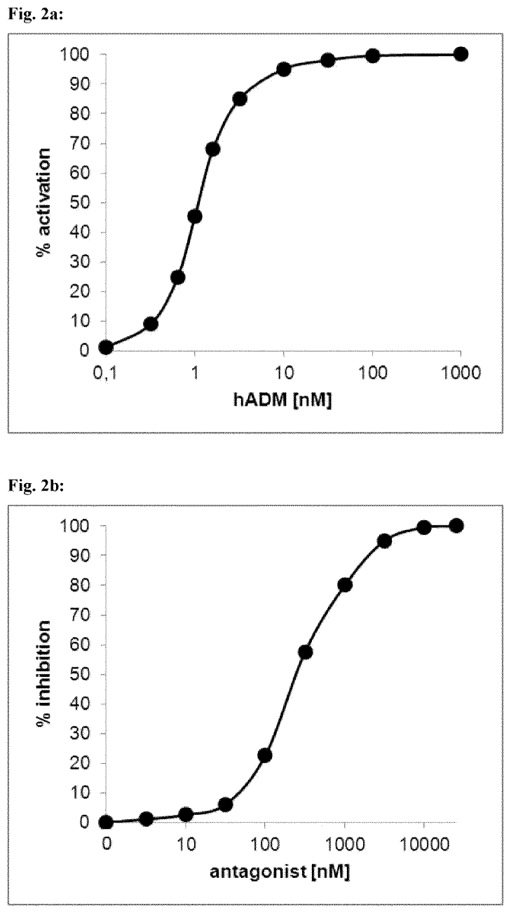

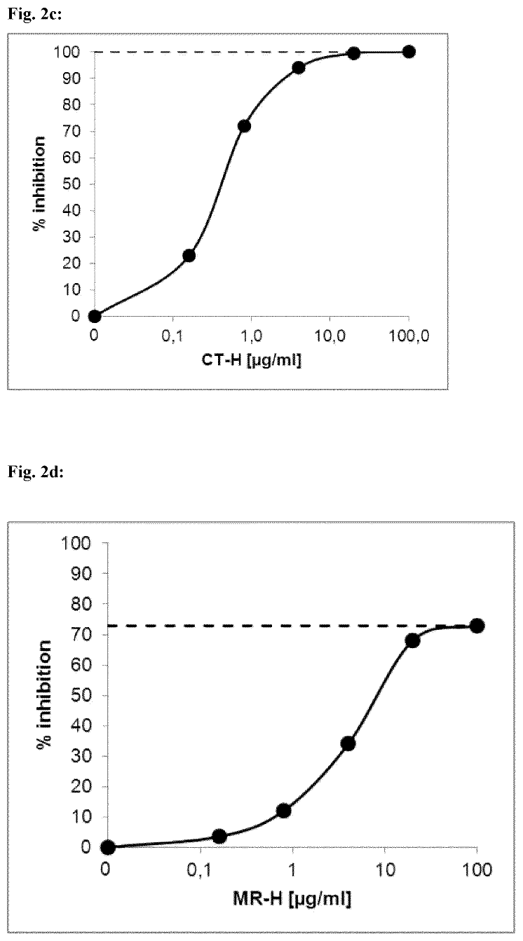

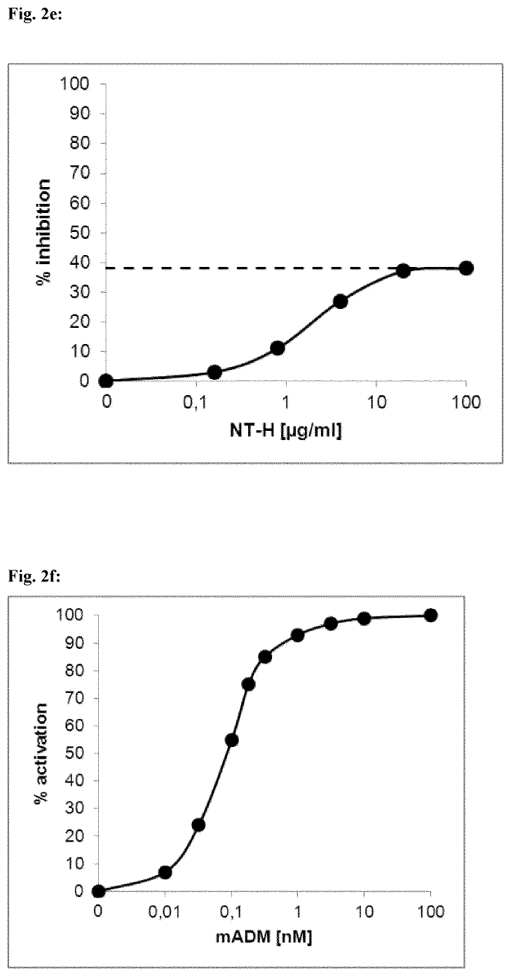

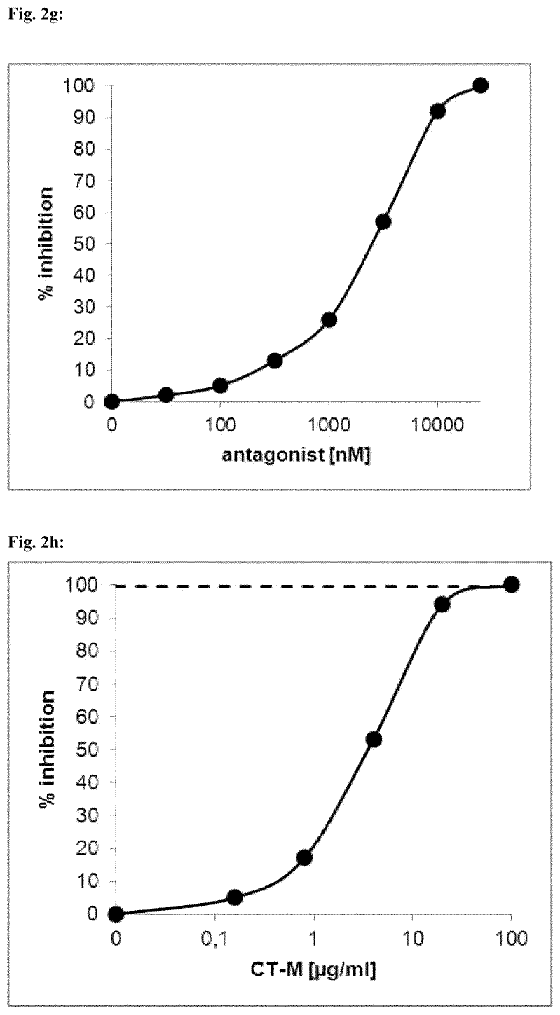

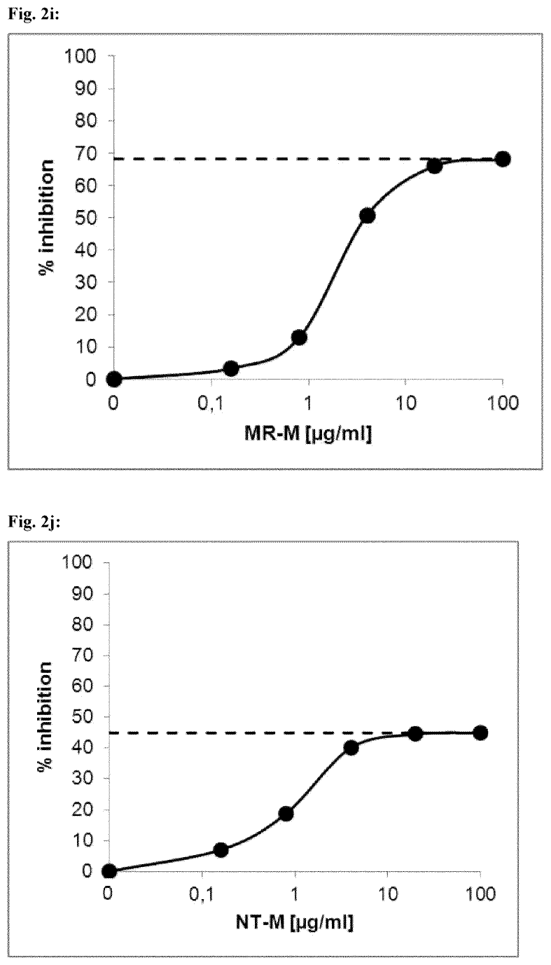

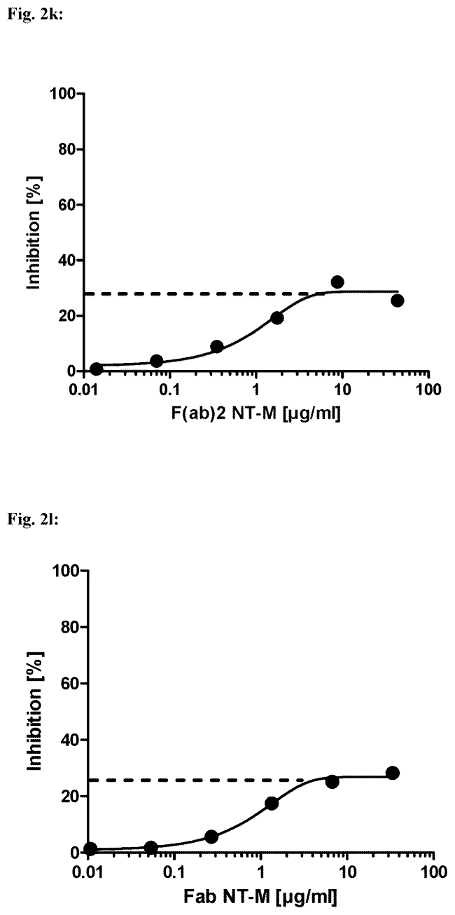

FIG. 2l: Efficacy of Adrecizumab treatment using different DPP3 threshold values: Kaplan-Meier survival plot (28-day mortality) for patients treated with placebo or the N-terminal-ADM antibody Adrecizumab with inclusion of patients with baseline DPP3 value below 50 ng/ml and DPP3<50 ng/mL in the following days.

SEQUENCES

SEQ ID No.: 1 (anti-ADM CDR 1 heavy chain)
GYTFSRYW
SEQ ID No.: 2 (anti-ADM CDR 2 heavy chain)
ILPGSGST
SEQ ID No.: 3 (anti-ADM CDR 3 heavy chain)
TEGYEYDGFDY
SEQ ID No.: 4 (anti-ADM CDR 1 light chain)
QSIVYSNGNTY
SEQUENCE “RVS” (anti-ADM CDR 2 light chain, not part of the Sequencing Listing):
RVS
SEQ ID No.: 5 (anti-ADM CDR 3 light chain)
FQGSHIPYT
SEQ ID No.: 6 (AM-VH-C)
QVQLQQSGAELMKPGASVKISCKATGYTFSRYWIEWVKQRPGHGLEWIGEILPGSGSTNYNE
KFKGKATITADTSSNTAYMQLSSLTSEDSAVYYCTEGYEYDGFDYWGQGTTLTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKRVEPK
SEQ ID No.: 7 (AM-VH1)
QVQLVQSGAEVKKPGSSVKVSCKASGYTFSRYWISWVRQAPGQGLEWMGRILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
SEQ ID No.: 8 (AM-VH2-E40)
QVQLVQSGAEVKKPGSSVKVSCKASGYTFSRYWIEWVRQAPGQGLEWMGRILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
SEQ ID No.: 9 (AM-VH3-T26-E55)
QVQLVQSGAEVKKPGSSVKVSCKATGYTFSRYWISWVRQAPGQGLEWMGEILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
SEQ ID No.: 10 (AM-VH4-T26-E40-E55)
QVQLVQSGAEVKKPGSSVKVSCKATGYTFSRYWIEWVRQAPGQGLEWMGEILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
SEQ ID No.: 11 (AM-VL-C)
DVLLSQTPLSLPVSLGDQATISCRSSQSIVYSNGNTYLEWYLQKPGQSPKLLIYRVSNRFSGVP
DRFSGSGSGTDFTLKISRVEAEDLGVYYCFQGSHIPYTFGGGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
SEQ ID No.: 12 (AM-VL1)
DVVMTQSPLSLPVTLGQPASISCRSSQSIVYSNGNTYLNWFQQRPGQSPRRLIYRVSNRDSGVP
DRFSGSGSGTDFTLKISRVEAEDVGVYYCFQGSHIPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
SEQ ID No.: 13 (AM-VL2-E40)
DVVMTQSPLSLPVTLGQPASISCRSSQSIVYSNGNTYLEWFQQRPGQSPRRLIYRVSNRDSGVP
DRFSGSGSGTDFTLKISRVEAEDVGVYYCFQGSHIPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
SEQ ID No.: 14 (human ADM 1-21)
YRQSMNNFQGLRSFGCRFGTC
SEQ ID No.: 15 (human ADM 21-32)
CTVQKLAHQIYQ
SEQ ID No.: 16 (human ADM C-42-52)
CAPRSKISPQGY-CONH2
SEQ ID No.: 17 (murine ADM 1-19)
YRQSMNQGSRSNGCRFGTC
SEQ ID No.: 18 (murine ADM 19-31)
CTFQKLAHQIYQ
SEQ ID No.: 19 (murine ADM C-40-50)
CAPRNKISPQGY-CONH₂
SEQ ID No.: 20 (mature human Adrenomedullin (mature ADM); amidated ADM; bio-ADM): amino
acids 1-52 or amino acids 95-146 of pro-ADM
YRQSMNNFQGLRSFGCRFGTCTVQKLAHQIYQFTDKDKDNVAPRSKISPQGY-CONH2
SEQ ID No.: 21 (Murine ADM 1-50)
YRQSMNQGSRSNGCRFGTCTFQKLAHQIYQLTDKDKDGMAPRNKISPQGY-CONH2
SEQ ID No.: 22 (1-21 of human ADM):
YRQSMNNFQGLRSFGCRFGTC
SEQ ID No.: 23 (1-42 of human ADM):
YRQSMNNFQGLRSFGCRFGTCTVQKLAHQIYQFTDKDKDNVA
SEQ ID No.: 24 (aa 43-52 of human ADM)
PRSKISPQGY-NH2
SEQ ID No.: 25 (aa 1-14 of human ADM)
YRQSMNNFQGLRSF
SEQ ID No.: 26 (aa 1-10 of human ADM)
YRQSMNNFQG
SEQ ID No.: 27 (aa 1-6 of human ADM)
YRQSMN
SEQ ID No.: 28 (aa 1-32 of human ADM)
YRQSMNNFQGLRSFGCRFGTCTVQKLAHQIYQ
SEQ ID No.: 29 (aa 1-40 murine ADM)
YRQSMNQGSRSNGCRFGTCTFQKLAHQIYQLTDKDKDGMA
SEQ ID No.: 30 (aa 1-31 murine ADM)
YRQSMNQGSRSNGCRFGTCTFQKLAHQIYQL
SEQ ID No.: 31 (proADM: 164 amino acids (22-185 of preproADM))
ARLDVASEF RKKWNKWALS RGKRELRMSS SYPTGLADVK AGPAQTLIRP QDMKGASRSP
EDSSPDAARI RVKRYRQSMN NFQGLRSFGC RFGTCTVQKL AHQIYQFTDK DKDNVAPRSK
ISPQGYGRRR RRSLPEAGPG RTLVSSKPQA HGAPAPPSGS APHFL
SEQ ID NO: 32 (Adrecizumab heavy chain)
QVQLVQSGAEVKKPGSSVKVSCKASGYTFSRYWIEWVRQAPGQGLEWIGEILPGSGSTNYNQ
KFQGRVTITADTSTSTAYMELSSLRSEDTAVYYCTEGYEYDGFDYWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYP
SDIAVEWESNGQPENNYKTTPPVLD
SDGSFFLYSKLTVDKSRWQQGNVFSCSVMHEALHNHYTQKSLSLSPGK
SEQ ID NO: 33 (Adrecizumab light chain)
DVVLTQSPLSLPVTLGQPASISCRSSQSIVYSNGNTYLEWYLQRPGQSPRLLIYRVSNRFSGVP
DRFSGSGSGTDFTLKISRVEAEDVGVYYCFQGSHIPYTFGGGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
SEQ ID No. 34-IGHV1-69*11
QVQLVQSGAEVKKPGSSVKVSCKASGGTFSSYAISWVRQAPGQGLEWMGRIIPILGTANYAQ
KFQGRVTITADESTSTAYMELSSLRSEDTAVYYCARYYYYYGMDVWGQGTTVTVSS
SEQ ID No. 35-HB3
QVQLQQSGAELMKPGASVKISCKATGYTFSRYWIEWVKQRPGHGLEWIGEILPGSGSTNYNE
KFKGKATITADTSSNTAYMQLSSLTSEDSAVYYCTEGYEYDGFDYWGQGTTLTVSS
SEQ ID No. 36-human DPP3 (amino acid 1-737)
MADTQYILPNDIGVSSLDCREAFRLLSPTERLYAYHLSRAAWYGGLAVLLQTSPEAPYIYALL
SRLFRAQDPDQLRQHALAEGLTEEEYQAFLVYAAGVYSNMGNYKSFGDTKFVPNLPKEKLE
RVILGSEAAQQHPEEVRGLWQTCGELMFSLEPRLRHLGLGKEGITTYFSGNCTMEDAKLAQD
FLDSQNLSAYNTRLFKEVDGEGKPYYEVRLASVLGSEPSLDSEVTSKLKSYEFRGSPFQVTRG
DYAPILQKVVEQLEKAKAYAANSHQGQMLAQYIESFTQGSIEAHKRGSRFWIQDKGPIVESYI
GFIESYRDPFGSRGEFEGFVAVVNKAMSAKFERLVASAEQLLKELPWPPTFEKDKFLTPDFTS
LDVLTFAGSGIPAGINIPNYDDLRQTEGFKNVSLGNVLAVAYATQREKLTFLEEDDKDLYILW
KGPSFDVQVGLHELLGHGSGKLFVQDEKGAFNFDQETVINPETGEQIQSWYRSGETWDSKFS
TIASSYEECRAESVGLYLCLHPQVLEIFGFEGADAEDVIYVNWLNMVRAGLLALEFYTPEAFN
WRQAHMQARFVILRVLLEAGEGLVTITPTTGSDGRPDARVRLDRSKIRSVGKPALERFLRRLQ
VLKSTGDVAGGRALYEGYATVTDAPPECFLTLRDTVLLRKESRKLIVQPNTRLEGSDVQLLE
YEASAAGLIRSFSERFPEDGPELEEILTQLATADARFWKGPSEAPSGQA
SEQ ID No. 37-human DPP3 (amino acid 474-493 (N-Cys))-immunization peptide with additional
N-terminal Cystein
CETVINPETGEQIQSWYRSGE
SEQ ID No. 38-hDPP3 aa 477-482-epitope of AK1967
INPETG
SEQ ID No. 39-hDPP3 aa 480-483
ETGE
SEQ ID No. 40-variable region of murine AK1967 in heavy chain
QVTLKESGPGILQPSQTLSLTCSFSGFSLSTSGMSVGWIRQPSGKGLEWLAHIWWNDNKSYNP
ALKSRLTISRDTSNNQVFLKIASVVTADTGTYFCARNYSYDYWGQGTTLTVSS
SEQ ID No. 41-variable region of murine AK1967 in light chain
DVVVTQTPLSLSVSLGDPASISCRSSRSLVHSIGSTYLHWYLQKPGQSPKLLIYKVSNRFSGVP
DRFSGSGSGTDFTLKISRVEAEDLGVYFCSQSTHVPWTFGGGTKLEIK
SEQ ID No. 42-CDR1 of murine AK1967 in heavy chain
GFSLSTSGMS
SEQ ID No. 43-CDR2 of murine AK1967 in heavy chain
IWWNDNK
SEQ ID No. 44-CDR 3 of murine AK1967 in heavy chain
ARNYSYDY
SEQ ID No. 45-CDR1 of murine AK1967 in light chain
RSLVHSIGSTY
CDR2 of murine AK1967 in light chain (without sequence ID)
KVS
SEQ ID No. 46-CDR3 of murine AK1967 in light chain
SQSTHVPWT
SEQ ID No. 47-humanized AK1967-heavy chain sequence (IgG1κ backbone)
MDPKGSLSWRILLFLSLAFELSYGQITLKESGPTLVKPTQTLTLTCTFSGFSLSTSGMSVGWIR
QPPGKALEWLAHIWWNDNKSYNPALKSRLTITRDTSKNQVVLTMTNMDPVDTGTYYCARN
YSYDYWGQGTLVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKKVEPKSCDKTHTC
PPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPG
SEQ ID No. 48-humanized AK1967-light chain sequence (IgG1κ backbone)
METDTLLLWVLLLWVPGSTGDIVMTQTPLSLSVTPGQPASISCKSSRSLVHSIGSTYLYWYLQ
KPGQSPQLLIYKVSNRFSGVPDRFSGSGSGTDFTLKISRVEAEDVGVYYCSQSTHVPWTFGGG
TKVEIKRTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC

For the avoidance of doubt, references to a C-terminal group “NH₂” and “CONH₂” likewise refer to a C-terminal amide group.

With the above context, the following consecutively numbered embodiments provide further specific aspects of the invention:

1. Method for the prediction of an increase of dipeptidyl peptidase 3 (DPP3) in a critical ill patient, the method comprising:
determining the level of DPP3 in a sample of bodily fluid of said patient,
comparing said determined level of DPP3 to a pre-determined threshold, wherein said threshold is in the range between 40 ng/ml and 22 ng/ml
wherein a level of DPP3 in said sample above said pre-determined threshold is indicative for an increase of DPP3 in said patient.

In certain embodiments, the method is for the prediction of an increase of dipeptidyl peptidase 3 (DPP3) in a critical ill patient, the method comprising:
determining the level of DPP3 in a sample of bodily fluid of said patient,
comparing said determined level of DPP3 to a pre-determined threshold, wherein said threshold is in the range between 40 ng/ml and 22 ng/ml
wherein a level of DPP3 in said sample above said pre-determined threshold is indicative for an increase of DPP3 in said patient during follow-up time.

2. Method for the prediction of an increase of DPP3 in a critical ill patient according to embodiment 1, wherein said pre-determined threshold is between 30 ng/ml and 22 ng/ml.

3. Method for the prediction of an increase of DPP3 in a critical ill patient according to embodiment 1 or 2, wherein said pre-determined threshold is between 25 ng/ml and 22 ng/ml.

4. Method for the prediction of an increase of DPP3 in a critical ill patient according to any of embodiments 1-3, wherein said predicted increase is an increase to DPP3 levels equal to or above 40, preferred equal to or above 50 ng/ml.

5. Method for the prediction of an increase of DPP3 in a critical ill patient according to embodiments 1-3, wherein said predicted increase of the DPP3 level is equal to or above 10%, more preferred equal to or above 20%, even more preferred equal to or above 40%, even more preferred equal to or above 50%, even more preferred equal to or above 75%, even more preferred equal to or above 100%,

6. Method for the prediction of an increase of DPP3 in a critical ill patient according to embodiments 1-5, wherein said increase of DPP3 is within up to 12 hours, preferably up to 24, 48, 72, 96 hours, more preferred up to 5 days, even more preferred up to 6 days, most preferred up to 7 days.

7. Method for the prediction of an increase of DPP3 in a critical ill patient according to embodiment 1-6, wherein said patient is a patient with severe infection, sepsis, heart failure, chronic heart failure, acute heart failure (AHF), myocardial infarction (MI), stroke, a patient with organ dysfunction or organ failure (e.g., dysfunction or failure of liver, kidney, lung), a patient undergoing major surgery, a patient with trauma (e.g. burn trauma, polytrauma), a patient with shock and/or a patient running into shock, or alternatively ARDS.

8. Method for the prediction of an increase of DPP3 in a critical ill patient according to embodiment 7, wherein said shock is selected from the group comprising shock due to hypovolemia, cardiogenic shock, obstructive shock and distributive shock.

9. Method for the prediction of an increase of DPP3 in a critical ill patient according to embodiment 8, wherein
in case of cardiogenic shock said patient may have suffered an acute coronary syndrome (e.g., acute myocardial infarction) or wherein said patient has heart failure (e.g., acute decompensated heart failure), myocarditis, arrhythmia, cardiomyopathy, valvular heart disease, aortic dissection with acute aortic stenosis, traumatic chordal rupture or massive pulmonary embolism, or
in case of hypovolemic shock said patient may have suffered a hemorrhagic disease including gastrointestinal bleed, trauma, vascular etiologies (e.g. ruptured abdominal aortic aneurysm, tumor eroding into a major blood vessel) and spontaneous bleeding in the setting of anticoagulant use or a non-hemorrhagic disease including vomiting, diarrhea, renal loss, skin losses/insensible losses (e.g., burns, heat stroke) or third-space loss in the setting of pancreatitis, cirrhosis, intestinal obstruction, or
in case of obstructive shock said patient may have suffered a cardiac tamponade, tension pneumothorax, pulmonary embolism or aortic stenosis, or
in case of distributive shock said patient may have septic shock, neurogenic shock, anaphylactic shock or shock due to adrenal crisis.

10. Method for the prediction of an increase of DPP3 in a critical ill patient according to any of embodiments 7-9, wherein said shock is selected from the group comprising cardiogenic shock or septic shock.

11. Method for the prediction of an increase of DPP3 in a critical ill patient according to any of embodiments 1-10, wherein a patient is selected for therapy/treatment if the level of a DPP3 in said sample is below said pre-determined threshold, wherein said therapy is selected from the group of alkaline phosphatase, immune suppressors, corticosteroids, vasopressors, fluids, anti-Adrenomedullin antibodies or antibody fragments or scaffolds.

12. Method for the prediction of an increase of DPP3 in a critical ill patient according to embodiment 11, wherein said anti-adrenomedullin antibodies or anti-adrenomedullin antibody fragments or anti-adrenomedullin scaffolds are directed to the N-terminal part (amino acids 1-21) of adrenomedullin (ADM): YRQSMNNFQGLRSFGCRFGTC (SEQ ID No. 14) 13. Method for the prediction of an increase of DPP3 in a critical ill patient according to any of embodiments 1-12, wherein a patient is selected for therapy/treatment with DPP3 inhibitors if the level of a DPP3 in said sample is above said pre-determined threshold, wherein said DPP3 inhibitor is selected from the group of anti-DPP3-antibodies or anti-DPP3-antibody fragments or anti-DPP3 scaffolds.

14. Method for the prediction of an increase of DPP3 in a critical ill patient according to any of embodiments 1-13, wherein said level of DPP3 is either the amount of DPP3 protein and/or the level of active DPP3.

15. Method for the prediction of an increase of DPP3 in a critical ill patient according to any of embodiments 1-14, wherein said level of DPP3 is determined by different methods, comprising an immunoassay, an activity assay or mass spectrometric methods.

16. Method for the prediction of an increase of DPP3 in a critical ill patient according to any of embodiment 15, wherein said immunoassay is a sandwich immunoassay.

17. Method for the prediction of an increase of DPP3 in a critical ill patient according to any of embodiments 1-16, wherein said bodily fluid is selected from whole blood, serum or plasma.

18. A method for the prevention of a DPP3 increase in a critical ill patient the method comprising:
determining the level of DPP3 in a sample of bodily fluid of said patient,
comparing said determined level of DPP3 to a pre-determined threshold, wherein said pre-determined threshold is between 40 ng/ml and 22 ng/ml and wherein a level of a DPP3 in said sample above said pre-determined is indicative for an increase of DPP3 in said patient, and
administering a DPP3 inhibitor if said determined level of DPP3 is above said pre-determined threshold,
wherein said DPP3 inhibitor is an anti-DPP3-antibody and/or and anti-DPP3-antibody fragment and/or anti-DPP3 scaffold.

19. A method for the prevention of a DPP3 increase in a critical ill patient according to embodiment 18, wherein said pre-determined threshold is between 30 ng/ml and 22 ng/ml.

20. A method for the prevention of a DPP3 increase in a critical ill patient according to embodiment 18 or 19, wherein said pre-determined threshold is between 25 ng/ml and 22 ng/ml.

21. A method for the prevention of a DPP3 increase in a critical ill patient according to any of embodiments 18-20, wherein said increase is an increase to DPP3 levels equal to or above 40, preferred equal to or above 50 ng/ml.

22. A method for the prevention of a DPP3 increase in a critical ill patient according to embodiment 18-21, wherein said patient is a patient with severe infection, sepsis, heart failure, chronic heart failure, acute heart failure (AHF), myocardial infarction (MI), stroke, a patient with organ dysfunction or organ failure (e.g., dysfunction or failure of liver, kidney, lung), a patient undergoing major surgery, a patient with trauma (e.g. burn trauma, polytrauma), a patient with shock and/or a patient running into shock, or alternatively ARDS.

23. A method for the prevention of a DPP3 increase in a critical ill patient according to embodiment 22, wherein said shock is selected from the group comprising shock due to hypovolemia, cardiogenic shock, obstructive shock and distributive shock.

24. A method for the prevention of a DPP3 increase in a critical ill patient according to embodiment 23, wherein
in case of cardiogenic shock said patient may have suffered an acute coronary syndrome (e.g., acute myocardial infarction) or wherein said patient has heart failure (e.g., acute decompensated heart failure), myocarditis, arrhythmia, cardiomyopathy, valvular heart disease, aortic dissection with acute aortic stenosis, traumatic chordal rupture or massive pulmonary embolism, or
in case of hypovolemic shock said patient may have suffered a hemorrhagic disease including gastrointestinal bleed, trauma, vascular etiologies (e.g. ruptured abdominal aortic aneurysm, tumor eroding into a major blood vessel) and spontaneous bleeding in the setting of anticoagulant use or a non-hemorrhagic disease including vomiting, diarrhea, renal loss, skin losses/insensible losses (e.g., burns, heat stroke) or third-space loss in the setting of pancreatitis, cirrhosis, intestinal obstruction, or
in case of obstructive shock said patient may have suffered a cardiac tamponade, tension pneumothorax, pulmonary embolism or aortic stenosis, or
in case of distributive shock said patient may have septic shock, neurogenic shock, anaphylactic shock or shock due to adrenal crisis.

25. A method for the prevention of a DPP3 increase in a critical ill patient according to any of embodiments 22-24, wherein said shock is selected from the group comprising cardiogenic shock or septic shock.

26. A DPP3 inhibitor for use in the prevention of a DPP3 increase in a critical ill patient, wherein said patient has a level of DPP3 above a threshold, wherein said threshold is between 40 ng/ml and 22 ng/ml and wherein said DPP3 inhibitor is an anti-DPP3-antibody and/or and anti-DPP3-antibody fragment and/or anti-DPP3 scaffold.

### EXAMPLES

### Example 1—Generation of Anti-ADM Antibodies and Determination of their Affinity Constants

Several human and murine antibodies were produced and their affinity constants were determined (see tables 1 and 2). It should be emphasized that the antibodies, antibody fragments and non-Ig scaffolds of the example portion in accordance with the invention are binding to ADM, and thus should be considered as anti-ADM antibodies/antibody fragments/non-Ig scaffolds.

### Peptides/Conjugates for Immunization:

Peptides for immunization were synthesized, see Table 1, (JPT Technologies, Berlin, Germany) with an additional N-terminal Cystein (if no Cystein is present within the selected ADM-sequence) residue for conjugation of the peptides to Bovine Serum Albumin (BSA). The peptides were covalently linked to BSA by using Sulfolink-coupling gel (Perbio-science, Bonn, Germany). The coupling procedure was performed according to the manual of Perbio.

### Mouse Monoclonal Antibody Production:

A Balb/c mouse was immunized with 100 μg Peptide-BSA-Conjugate at day 0 and 14 (emulsified in 100 μl complete Freund's adjuvant) and 50 μg at day 21 and 28 (in 100 μl incomplete Freund's adjuvant). Three days before the fusion experiment was performed, the animal received 50 μg of the conjugate dissolved in 100 μl saline, given as one intraperitoneal and one intra-venous injection. Splenocytes from immunized mouse and cells of the myeloma cell line SP2/0 were fused with 1 ml 50% polyethylene glycol for 30 s at 37° C. After washing, the cells were seeded in 96-well cell culture plates. Hybrid clones were selected by growing in HAT medium [RPMI 1640 culture medium supplemented with 20% fetal calf serum and HAT-Supplement]. After two weeks the HAT medium is replaced with HT Medium for three passages followed by returning to the normal cell culture medium. Cell culture supernatants were primary screened for antigen specific IgG antibodies three weeks after fusion. The positive tested microcultures were transferred into 24-well plates for propagation. After retesting, the selected cultures were cloned and re-cloned using the limiting-dilution technique and isotypes were determined (see also Lane, R. D. 1985. J. Immunol. Meth. 81: 223-228; Ziegler et al. 1996. Horm. Metab. Res. 28: 11-15).

Antibodies were produced via standard antibody production methods (Marx et al, 1997. Monoclonal Antibody Production, ATLA 25, 121) and purified via Protein A. The antibody purities were >95% based on SDS gel electrophoresis analysis.

### Human Antibodies:

Human Antibodies were produced by means of phage display according to the following procedure: The human naive antibody gene libraries HAL7/8 were used for the isolation of recombinant single chain F-Variable domains (scFv) against adrenomedullin peptide. The antibody gene libraries were screened with a panning strategy comprising the use of peptides containing a biotin tag linked via two different spacers to the adrenomedullin peptide sequence. A mix of panning rounds using non-specifically bound antigen and streptavidin bound antigen were used to minimize background of non-specific binders. The eluted phages from the third round of panning have been used for the generation of monoclonal scFv expressing E. coli strains. Supernatant from the cultivation of these clonal strains has been directly used for an antigen ELISA testing (see also Hust et al. 2011. Journal of Biotechnology 152, 159-170; Schütte et al. 2009. PLoS One 4, e6625). Positive clones have been selected based on positive ELISA signal for antigen and negative for streptavidin coated micro titer plates. For further characterizations the scFv open reading frame has been cloned into the expression plasmid pOPE107 (Hust et al., J. Biotechn. 2011), captured from the culture supernatant via immobilized metal ion affinity chromatography and purified by a size exclusion chromatography.

Affinity Constants: To determine the affinity of the antibodies to ADM, the kinetics of binding of ADM to immobilized antibody was determined by means of label-free surface plasmon resonance using a Biacore 2000 system (GE Healthcare Europe GmbH, Freiburg, Germany). Reversible immobilization of the antibodies was performed using an anti-mouse Fc antibody covalently coupled in high density to a CM5 sensor surface according to the manufacturer's instructions (mouse antibody capture kit; GE Healthcare). (Lorenz et al. 2011. Antimicrob Agents Chemother. 55(1): 165-173).

The monoclonal antibodies were raised against the below depicted ADM regions of human and murine ADM, respectively. The following table represents a selection of obtained antibodies used in further experiments. Selection was based on target region:

TABLE 1
ADM immunization peptides

| | | | | Affinity |
|---|---|---|---|---|
| Sequence | | ADM | | constants |
| Number | Antigen/Immunogen | Region | Designation | Kd (M) |
| SEQ ID: 14 | YRQSMNNFQGLRSFGCRFGTC | 1-21 | NT-H | 5.9 × 10⁻⁹ |
| SEQ ID: 15 | CTVQKLAHQIYQ | 21-32 | MR-H | 2 × 10⁻⁹ |
| SEQ ID: 16 | CAPRSKISPQGY-NH₂ | C-42-52 | CT-H | 1.1 × 10⁻⁹ |
| SEQ ID: 17 | YRQSMNQGSRSNGCRFGTC | 1-19 | NT-M | 3.9 ×x 10⁻⁹ |
| SEQ ID: 18 | CTFQKLAHQIYQ | 19-31 | MR-M | 4.5 x 10⁻¹⁰ |
| SEQ ID: 19 | CAPRNKISPQGY-NH₂ | C-40-50 | CT-M | 9 × 10⁻⁹ |

TABLE 2
Further obtained monoclonal anti-ADM antibodies

| | | | | max inhibition |
|---|---|---|---|---|
| | | Clone | Affinity | bioassay (%) |
| Target | Source | number | (M) | (see example 2) |
| NT-M | Mouse | ADM/63 | 5.8 × 10⁻⁹ | 45 |
| | Mouse | ADM/364 | 2.2 × 10⁻⁸ | 48 |
| | Mouse | ADM/365 | 3.0 × 10⁻⁸ | |
| | Mouse | ADM/366 | 1.7 × 10⁻⁸ | |
| | Mouse | ADM/367 | 1.3 × 10⁻⁸ | |
| | Mouse | ADM/368 | 1.9 × 10⁻⁸ | |
| | Mouse | ADM/369 | 2.0 × 10⁻⁸ | |
| | Mouse | ADM/370 | 1.6 × 10⁻⁸ | |
| | Mouse | ADM/371 | 2.0 × 10⁻⁸ | |
| | Mouse | ADM/372 | 2.5 × 10⁻⁸ | |
| | Mouse | ADM/373 | 1.8 × 10⁻⁸ | |
| | Mouse | ADM/377 | 1.5 × 10⁻⁸ | |
| | Mouse | ADM/378 | 2.2 × 10⁻⁸ | |
| | Mouse | ADM/379 | 1.6 × 10⁻⁸ | |
| | Mouse | ADM/380 | 1.8 × 10⁻⁸ | |
| | Mouse | ADM/381 | 2.4 × 10⁻⁸ | |
| | Mouse | ADM/382 | 1.6 × 10⁻⁸ | |
| | Mouse | ADM/383 | 1.8 × 10⁻⁸ | |
| | Mouse | ADM/384 | 1.7 × 10⁻⁸ | |
| | Mouse | ADM/385 | 1.7 × 10⁻⁸ | |
| | Mouse | ADM/403 | 1.2 × 10⁻⁸ | |
| | Mouse | ADM/395 | 1.2 × 10⁻⁸ | |
| | Mouse | ADM/396 | 3.0 × 10⁻⁸ | |
| | Mouse | ADM/397 | 1.5 × 10⁻⁸ | |
| MR-M | Mouse | ADM/38 | 4.5 × 10⁻¹⁰ | 68 |
| MR-M | Mouse | ADM/39 | 5.9 × 10⁻⁹ | 72 |
| CT-M | Mouse | ADM/65 | 9.0 × 10⁻⁹ | 100 |
| CT-M | Mouse | ADM/66 | 1.6 × 10⁻⁸ | 100 |
| NT-H | Mouse | ADM/33 | 5.9 × 10⁻⁸ | 38 |
| NT-H | Mouse | ADM/34 | 1.6 × 10⁻⁸ | 22 |
| MR-H | Mouse | ADM/41 | 1.2 × 10⁻⁸ | 67 |
| MR-H | Mouse | ADM/42 | <1 × 10⁻⁸ | |
| MR-H | Mouse | ADM/43 | 2.0 × 10⁻⁹ | 73 |
| MR-H | Mouse | ADM/44 | <1 × 10⁻⁸ | |
| CT-H | Mouse | ADM/15 | <1 × 10⁻⁸ | |
| CT-H | Mouse | ADM/16 | 1.1 × 10⁻⁹ | 100 |
| CT-H | Mouse | ADM/17 | 3.7 × 10⁻⁹ | 100 |
| CT-H | Mouse | ADM/18 | <1 × 10⁻⁸ | |
| hADM | Phage display | ADM/A7 | <1 × 10⁻⁸ | |
| | Phage display | ADM/B7 | <1 × 10⁻⁸ | |
| | Phage display | ADM/C7 | <1 × 10⁻⁸ | |
| | Phage display | ADM/G3 | <1 × 10⁻⁸ | |
| | Phage display | ADM/B6 | <1 × 10⁻⁸ | |
| | Phage display | ADM/B11 | <1 × 10⁻⁸ | |
| | Phage display | ADM/D8 | <1 × 10⁻⁸ | |
| | Phage display | ADM/D11 | <1 × 10⁻⁸ | |
| | Phage display | ADM/G12 | <1 × 10⁻⁸ | |

Generation of antibody fragments by enzymatic digestion: The generation of Fab and F(ab)₂ fragments was done by enzymatic digestion of the murine full-length antibody NT-M. Antibody NT-M was digested using a) the pepsin-based F(ab)₂ Preparation Kit (Pierce 44988) and b) the papain-based Fab Preparation Kit (Pierce 44985). The fragmentation procedures were performed according to the instructions provided by the supplier. Digestion was carried out in case of F(ab)₂-fragmentation for 8 h at 37° C. The Fab-fragmentation digestion was carried out for 16 h, respectively.

Procedure for Fab Generation and Purification: The immobilized papain was equilibrated by washing the resin with 0.5 ml of Digestion Buffer and centrifuging the column at 5000×g for 1 minute. The buffer was discarded afterwards. The desalting column was prepared by removing the storage solution and washing it with digestion buffer, centrifuging it each time afterwards at 1000×g for 2 minutes. 0.5 ml of the prepared IgG sample were added to the spin column tube containing the equilibrated Immobilized Papain. Incubation time of the digestion reaction was done for 16 h on a tabletop rocker at 37° C. The column was centrifuged at 5000×g for 1 minute to separate digest from the Immobilized Papain. Afterwards the resin was washed with 0.5 ml PBS and centrifuged at 5000×g for 1 minute. The wash fraction was added to the digested antibody that the total sample volume was 1.0 ml. The NAb Protein A Column was equilibrated with PBS and IgG Elution Buffer at room temperature. The column was centrifuged for 1 minute to remove storage solution (contains 0.02% sodium azide) and equilibrated by adding 2 ml of PBS, centrifuge again for 1 minute and the flow-through discarded. The sample was applied to the column and resuspended by inversion. Incubation was done at room temperature with end-over-end mixing for 10 minutes. The column was centrifuged for 1 minute, saving the flow-through with the Fab fragments. (References: Coulter and Harris 1983. J. Immunol. Meth. 59, 199-203.; Lindner et al. 2010. Cancer Res. 70, 277-87; Kaufmann et al. 2010. PNAS. 107, 18950-5.; Chen et al. 2010. PNAS. 107, 14727-32; Uysal et al. 2009 J. Exp. Med. 206, 449-62; Thomas et al. 2009. J. Exp. Med. 206, 1913-27; Kong et al. 2009 J. Cell Biol. 185, 1275-840).

Procedure for generation and purification of F(ab′)₂ Fragments: The immobilized Pepsin was equilibrated by washing the resin with 0.5 ml of Digestion Buffer and centrifuging the column at 5000×g for 1 minute. The buffer was discarded afterwards. The desalting column was prepared by removing the storage solution and washing it with digestion buffer, centrifuging it each time afterwards at 1000×g for 2 minutes. 0.5 ml of the prepared IgG sample were added to the spin column tube containing the equilibrated Immobilized Pepsin. Incubation time of the digestion reaction was done for 16 h on a tabletop rocker at 37° C. The column was centrifuged at 5000×g for 1 minute to separate digest from the Immobilized Papain. Afterwards the resin was washed with 0.5 mL PBS and centrifuged at 5000×g for 1 minute. The wash fraction was added to the digested antibody that the total sample volume was 1.0 ml. The NAb Protein A Column was equilibrated with PBS and IgG Elution Buffer at room temperature. The column was centrifuged for 1 minute to remove storage solution (contains 0.02% sodium azide) and equilibrated by adding 2 mL of PBS, centrifuge again for 1 minute and the flow-through discarded. The sample was applied to the column and resuspended by inversion. Incubation was done at room temperature with end-over-end mixing for 10 minutes. The column was centrifuged for 1 minute, saving the flow-through with the Fab fragments. (References: Mariani et al. 1991. Mol. Immunol. 28: 69-77; Beale 1987. Exp Comp Immunol 11:287-96; Ellerson et al. 1972. FEBS Letters 24(3):318-22; Kerbel and Elliot 1983. Meth Enzymol 93:113-147; Kulkarni et al. 1985. Cancer Immunol Immunotherapy 19:211-4; Lamovi 1986. Meth Enzymol 121:652-663; Parham et al. 1982. J Immunol Meth 53:133-73; Raychaudhuri et al. 1985. Mol Immunol 22(9):1009-19; Rousseaux et al. 1980. Mol Immunol 17:469-82; Rousseaux et al. 1983. J Immunol Meth 64:141-6; Wilson et al. 1991. J Immunol Meth 138:111-9).

NT-H-Antibody Fragment Humanization: The antibody fragment was humanized by the CDR-grafting method (Jones et al. 1986. Nature 321, 522-525).

The following steps were done to achieve the humanized sequence:

Total RNA extraction: Total RNA was extracted from NT-H hybridomas using the Qiagen kit. First-round RT-PCR: QIAGEN® OneStep RT-PCR Kit (Cat No. 210210) was used. RT-PCR was performed with primer sets specific for the heavy and light chains. For each RNA sample, 12 individual heavy chain and 11 light chain RT-PCR reactions were set up using degenerate forward primer mixtures covering the leader sequences of variable regions. Reverse primers are located in the constant regions of heavy and light chains. No restriction sites were engineered into the primers.

Reaction Setup: 5×QIAGEN® OneStep RT-PCR Buffer 5.0 μl, dNTP Mix (containing 10 mM of each dNTP) 0.8 μl, Primer set 0.5 μl, QIAGEN® OneStep RT-PCR Enzyme Mix 0.8 μl, Template RNA 2.0 μl, RNase-free water to 20.0 μl, Total volume 20.0 μl PCR condition: Reverse transcription: 50° C., 30 min; Initial PCR activation: 95° C., 15 min Cycling: 20 cycles of 94° C., 25 sec; 54° C., 30 sec; 72° C., 30 sec; Final extension: 72° C., 10 min Second-round semi-nested PCR: The RT-PCR products from the first-round reactions were further amplified in the second-round PCR. 12 individual heavy chain and 11 light chain RT-PCR reactions were set up using semi-nested primer sets specific for antibody variable regions.

Reaction Setup: 2×PCR mix 10 μl; Primer set 2 μl; First-round PCR product 8 μl; Total volume 20 μl; Hybridoma Antibody Cloning Report PCR condition: Initial denaturing of 5 min at 95° C.; 25 cycles of 95° C. for 25 sec, 57° C. for 30 sec, 68° C. for 30 sec; Final extension is 10 min 68° C.

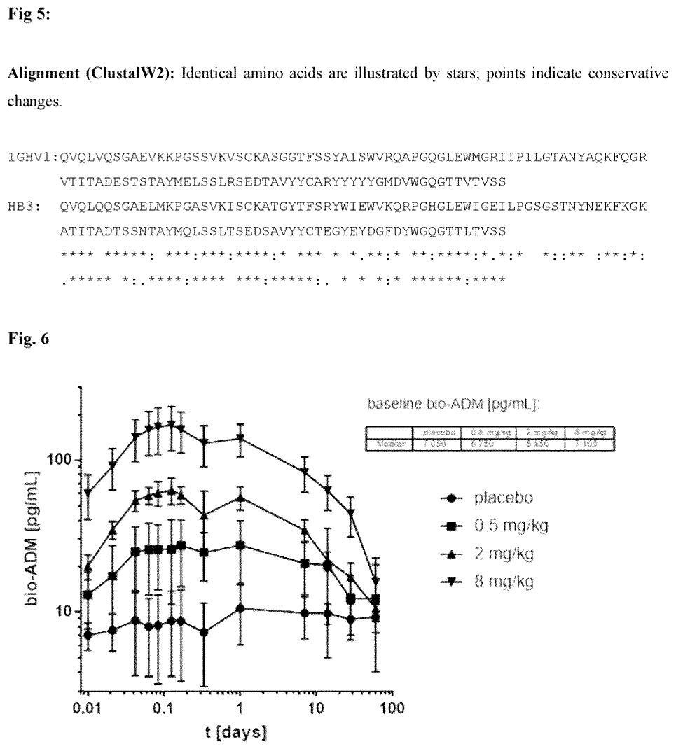

After PCR was finished, PCR reaction samples were run onto agarose gel to visualize DNA fragments amplified. After sequencing more than 15 cloned DNA fragments amplified by nested RT-PCR, several mouse antibody heavy and light chains have been cloned and appear correct. Protein sequence alignment and CDR analysis identifies one heavy chain and one light chain. After alignment with homologous human framework sequences the resulting humanized sequence for the variable heavy chain is the following: see FIG. 5. As the amino acids on positions 26, 40 and 55 in the variable heavy chain and amino acid on position 40 in the variable light are critical to the binding properties, they may be reverted to the murine original. The resulting candidates are depicted below. (Padlan 1991. Mol. Immunol. 28, 489-498; Harris and Bajorath. 1995. Protein Sci. 4, 306-310).

Annotation for the antibody fragment sequences (SEQ ID No.: 6-13; 32 and 33): bold and underline are the CDR 1, 2, 3 chronologically arranged; italic are constant regions; hinge regions are highlighted with bold letters; framework point mutation have a grey letter-background.

(AM-VH-C)
SEQ ID No.: 6
QVQLQQSGAELMKPGASVKISCKATGYTFSRYWIEWVKQRPGHGLEWIGEILPGSGSTNYN
EKFKGKATITADTSSNTAYMQLSSLTSEDSAVYYCTEGYEYDGFDYWGQGTTLTVSSASTKGPSVF
PLAPSSKSTSGGTAALGCLVKDYFPEPVTVSWNSGALTSGVHTFPAVLQSSGLYSLSSVVTVPSSSLGT
QTYICNVNHKPSNTKVDKRVEPK
(AM-VH1)
SEQ ID No.: 7
QVQLVQSGAEVKKPGSSVKVSCKASGYTFSRYWISWVRQAPGQGLEWMGRILPGSGSTNY
AQKFQGRVTITADESTSTAYMELSSLRSEDTAVYYCTEGYEYDGFDYWGQGTTVTVSSASTKGPSV
FPLAPSSKSTSGGTAALGCLVKDYFPEPVTVSWNSGALTSGVHTFPAVLQSSGLYSLSSVVTVPSSSLG
TQTYICNVNHKPSNTKVDKRVEPK
(AM-VH2-E40)
SEQ ID No.: 8
QVQLVQSGAEVKKPGSSVKVSCKASGYTFSRYWIEWVRQAPGQGLEWMGRILPGSGSTNY
AQKFQGRVTITADESTSTAYMELSSLRSEDTAVYYCTEGYEYDGFDYWGQGTTVTVSSASTKGPSV
FPLAPSSKSTSGGTAALGCLVKDYFPEPVTVSWNSGALTSGVHTFPAVLQSSGLYSLSSVVTVPSSSLG
TQTYICNVNHKPSNTKVDKRVEPK
(AM-VH3-T26-E55)
SEQ ID No.: 9
QVQLVQSGAEVKKPGSSVKVSCKATGYTFSRYWISWVRQAPGQGLEWMGEILPGSGSTNY
AQKFQGRVTITADESTSTAYMELSSLRSEDTAVYYCTEGYEYDGFDYWGQGTTVTVSSASTKGPSV
FPLAPSSKSTSGGTAALGCLVKDYFPEPVTVSWNSGALTSGVHTFPAVLQSSGLYSLSSVVTVPSSSLG
TQTYICNVNHKPSNTKVDKRVEPK
(AM-VH4-T26-E40-E55)
SEQ ID No.: 10
QVQLVQSGAEVKKPGSSVKVSCKATGYTFSRYWIEWVRQAPGQGLEWMGEILPGSGSTNY
AQKFQGRVTITADESTSTAYMELSSLRSEDTAVYYCTEGYEYDGFDYWGQGTTVTVSSASTKGPSV
FPLAPSSKSTSGGTAALGCLVKDYFPEPVTVSWNSGALTSGVHTFPAVLQSSGLYSLSSVVTVPSSSLG
TQTYICNVNHKPSNTKVDKRVEPK
(AM-VL-C)
SEQ ID No.: 11
DVLLSQTPLSLPVSLGDQATISCRSSQSIVYSNGNTYLEWYLQKPGQSPKLLIYRVSNRFSGVP
DRFSGSGSGTDFTLKISRVEAEDLGVYYCFQGSHIPYTFGGGTKLEIKRTVAAPSVFIFPPSDE
QLKSGTASVVCLLNNFYPREAKVQWKVDNALQSGNSQESVTEQDSKDSTYSLSSTLTLSKAD
YEKHKVYACEVTHQGLSSPVTKSFNRGEC
(AM-VL1)
SEQ ID No.: 12
DVVMTQSPLSLPVTLGQPASISCRSSQSIVYSNGNTYLNWFQQRPGQSPRRLIYRVSNRDSGV
PDRFSGSGSGTDFTLKISRVEAEDVGVYYCFQGSHIPYTFGQGTKLEIKRTVAAPSVFIFPPSD
EQLKSGTASVVCLLNNFYPREAKVQWKVDNALQSGNSQESVTEQDSKDSTYSLSSTLTLSKA
DYEKHKVYACEVTHQGLSSPVTKSFNRGEC
(AM-VL2-E40)
SEQ ID No.: 13
DVVMTQSPLSLPVTLGQPASISCRSSQSIVYSNGNTYLEWFQQRPGQSPRRLIYRVSNRDSGV
PDRFSGSGSGTDFTLKISRVEAEDVGVYYCFQGSHIPYTFGQGTKLEIKRTVAAPSVFIFPPSD
EQLKSGTASVVCLLNNFYPREAKVQWKVDNALQSGNSQESVTEQDSKDSTYSLSSTLTLSKA
DYEKHKVYACEVTHQGLSSPVTKSFNRGEC
(Adrecizumab heavy chain)
SEQ ID NO: 32
QVQLVQSGAEVKKPGSSVKVSCKASGYTFSRYWIEWVRQAPGQGLEWIGEILPGSGSTNYN
QKFQGRVTITADTSTSTAYMELSSLRSEDTAVYYCTEGYEYDGFDYWGQGTTVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALH
NHYTQKSLSLSPGK
(Adrecizumab light chain)
SEQ ID NO: 33
DVVLTQSPLSLPVTLGQPASISCRSSQSIVYSNGNTYLEWYLQRPGQSPRLLIYRVSNRFSGVP
DRFSGSGSGTDFTLKISRVEAEDVGVYYCFQGSHIPYTFGGGTKLEIKRTVAAPSVFIFPPSDE
QLKSGTASVVCLLNNFYPREAKVQWKVDNALQSGNSQESVTEQDSKDSTYSLSSTLTLSKAD
YEKHKVYACEVTHQGLSSPVTKSFNRGEC

### Example 2—Effect of Selected Anti-ADM-Antibodies on Anti-ADM-Bioactivity

The effect of selected ADM-antibodies on ADM-bioactivity was tested in a human recombinant Adrenomedullin receptor cAMP functional assay (Adrenomedullin Bioassay).

Testing of Antibodies Targeting Human or Mouse Adrenomedullin in Human Recombinant Adrenomedullin Receptor cAMP Functional Assay (Adrenomedullin Bioassay)

Materials: Cell line CHO-K1, Receptor Adrenomedullin (CRLR+RAMP3), Receptor Accession Number Cell line: CRLR: U17473; RAMP3: AJ001016

CHO-K1 cells expressing human recombinant adrenomedullin receptor (FAST-027C) grown prior to the test in media without antibiotic were detached by gentle flushing with PBS-EDTA (5 mM EDTA), recovered by centrifugation and resuspended in assay buffer (KRH: 5 mM KCl, 1.25 mM MgSO₄, 124 mM NaCl, 25 mM HEPES, 13.3 mM Glucose, 1.25 mM KH₂PO₄, 1.45 mM CaCl₂, 0.5 g/l BSA). Dose response curves were performed in parallel with the reference agonists (hADM or mADM).

Antagonist test (96 well): For antagonist testing, 6 μl of the reference agonist (human (5.63 nM) or mouse (0.67 nM) adrenomedullin) was mixed with 6 μl of the test samples at different antagonist dilutions; or with 6 μl buffer. After incubation for 60 min at room temperature, 12 μl of cells (2,500 cells/well) were added. The plates were incubated for 30 min at room temperature. After addition of the lysis buffer, percentage of DeltaF will be estimated, according to the manufacturer specification, with the HTRF kit from Cis-Bio International (cat n° 62AM2 PEB) hADM 22-52 was used as reference antagonist.

Antibodies Testing cAMP-HTRF Assay

The anti-h-ADM antibodies (NT-H, MR-H, CT-H) were tested for antagonist activity in human recombinant adrenomedullin receptor (FAST-027C) cAMP functional assay in the presence of 5.63 nM Human ADM 1-52, at the following final antibody concentrations: 100 g/ml, 20 μg/ml, 4 μg/ml, 0.8 μg/ml, 0.16 μg/ml.

The anti-m-ADM antibodies (NT-M, MR-M, CT-M) were tested for antagonist activity in human recombinant ADM receptor (FAST-027C) cAMP functional assay in the presence of 0.67 nM Mouse ADM 1-50, at the following final antibody concentrations: 100 μg/ml, 20 μg/ml, 4 μg/ml, 0.8 μg/ml, 0.16 μg/ml. Data were plotted relative inhibition vs. antagonist concentration (see FIGS. 2 a to 2 l). The maximal inhibition by the individual antibody is given in table 3.

TABLE 3
maximal inhibition of bio-ADM activity

| | Maximal inhibition of ADM bioactivity |
|---|---|
| Antibody | (ADM-Bioassay) (%) |
| NT-H | 38 |
| MR-H | 73 |
| CT-H | 100 |
| NT-M FAB | 26 |
| NT-M FAB2 | 28 |
| NT-M | 45 |
| MR-M | 66 |
| CT-M | 100 |
| Non specific mouse IgG | 0 |

### Example 3—Stabilization of hADM by the Anti-ADM Antibody

The stabilizing effect of human ADM by human ADM antibodies was tested using a hADM immunoassay.

### Immunoassay for the Quantification of Human Adrenomedullin

The technology used was a sandwich coated tube luminescence immunoassay, based on Acridinium ester labelling.

Labelled compound (tracer): 100 μg (100 μl) CT-H (1 mg/ml in PBS, pH 7.4, AdrenoMed AG Germany) was mixed with 10 μl Acridinium NHS-ester (1 mg/ml in acetonitrile, InVent GmbH, Germany) (EP 0353971) and incubated for 20 min at room temperature. Labelled CT-H was purified by Gel-filtration HPLC on Bio-Sil® SEC 400-5 (Bio-Rad Laboratories, Inc., USA) The purified CT-H was diluted in (300 mmol/L potassium phosphate, 100 mmol/L NaCl, 10 mmol/L Na-EDTA, 5 g/L Bovine Serum Albumin, pH 7.0). The final concentration was approx. 800.000 relative light units (RLU) of labelled compound (approx. 20 ng labeled antibody) per 200 μL. Acridiniumester chemiluminescence was measured by using an AutoLumat LB 953 (Berthold Technologies GmbH & Co. KG).

Solid phase: Polystyrene tubes (Greiner Bio-One International AG, Austria) were coated (18 h at room temperature) with MR-H (AdrenoMed AG, Germany) (1.5 g MR-H/0.3 mL 100 mmol/L NaCl, 50 mmol/L TRIS/HCl, pH 7.8). After blocking with 5% bovine serum albumin, the tubes were washed with PBS, pH 7.4 and vacuum dried.

Calibration: The assay was calibrated, using dilutions of hADM (BACHEM AG, Switzerland) in 250 mmol/L NaCl, 2 g/L Triton X-100, 50 g/L Bovine Serum Albumin, 20 tabs/L Protease Inhibitor Cocktail (Roche Diagnostics AG, Switzerland).

hADM Immunoassay: 50 μl of sample (or calibrator) was pipetted into coated tubes, after adding labeled CT-H (200 μl), the tubes were incubated for 4 h at 4° C. Unbound tracer was removed by washing 5 times (each 1 ml) with washing solution (20 mM PBS, pH 7.4, 0.1% Triton X-100).

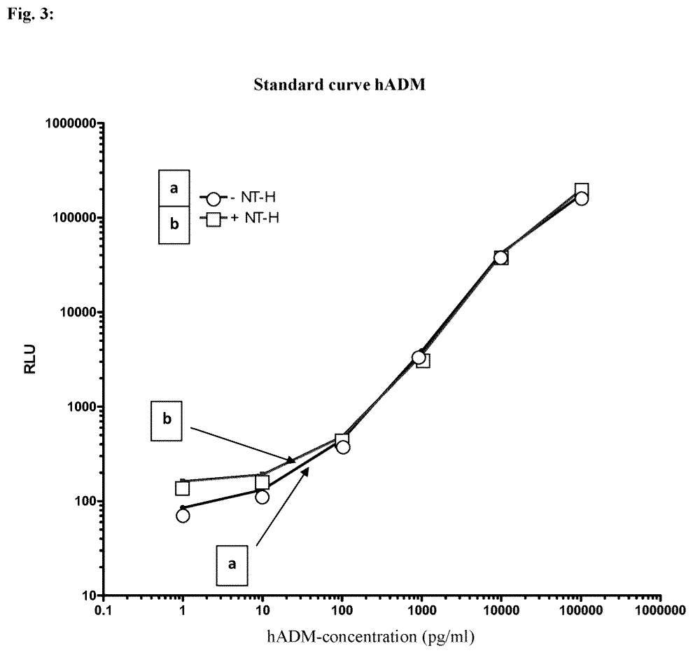

Tube-bound chemiluminescence was measured by using the LB 953: FIG. 3 shows a typical hADM dose/signal curve. And an hADM dose signal curve in the presence of 100 μg/mL antibody NT-H. NT-H did not affect the described hADM immunoassay.

Stability of human Adrenomedullin: Human ADM was diluted in human Citrate plasma (final concentration 10 nM) and incubated at 24° C. At selected time points, the degradation of hADM was stopped by freezing at −20° C. The incubation was performed in absence and presence of NT-H (100 μg/ml). The remaining hADM was quantified by using the hADM immunoassay described above.

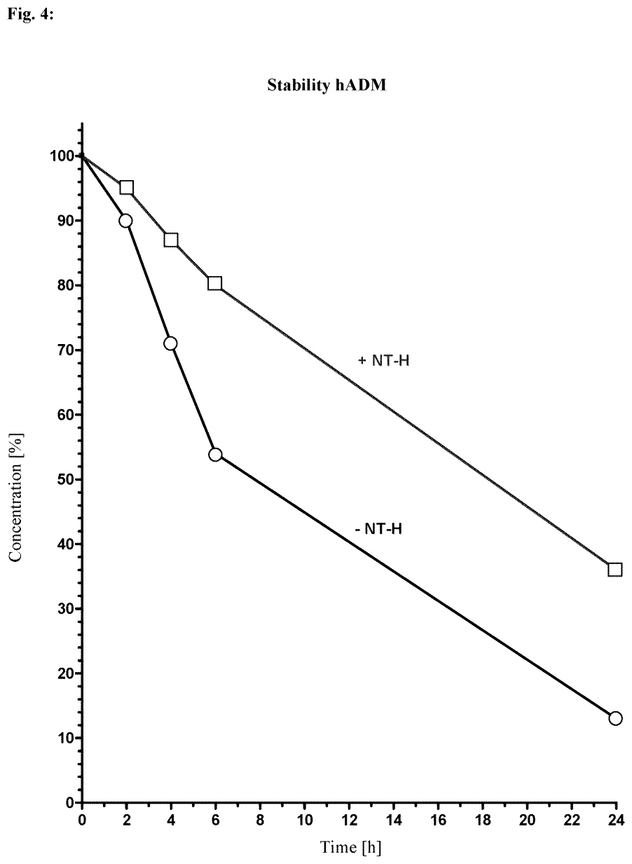

FIG. 4 shows the stability of hADM in human plasma (citrate) in absence and in the presence of NT-H antibody. The half-life of hADM alone was 7.8 h and in the presence of NT-H, the half-life was 18.3 h. (2.3 times higher stability).

### Example 4—Sepsis Mortality in Mice Treated with Anti-ADM Antibodies

### 4) Early Treatment of Sepsis

Animal model: 12-15 week-old male C57Bl/6 mice (Charles River Laboratories, Germany) were used for the study. Peritonitis had been surgically induced under light 79soflurane anesthesia. Incisions were made into the left upper quadrant of the peritoneal cavity (normal location of the cecum). The cecum was exposed and a tight ligature was placed around the cecum with sutures distal to the insertion of the small bowel. One puncture wound was made with a 24-gauge needle into the cecum and small amounts of cecal contents were expressed through the wound. The cecum was replaced into the peritoneal cavity and the laparotomy site was closed. Finally, animals were returned to their cages with free access to food and water. 500 μl saline were given s.c. as fluid replacement.

Application and dosage of the compound (NT-M, MR-M, CT-M): Mice were treated immediately after CLP (early treatment). CLP is the abbreviation for cecal ligation and puncture (CLP).

Study groups: Three compounds were tested versus: vehicle and versus control compound treatment. Each group contained 5 mice for blood drawing after 1 day for BUN (serum blood urea nitrogen test) determination. Ten further mice per each group were followed over a period of 4 days.

Group Treatment (10 μl/g bodyweight) dose/Follow-Up:
1 NT-M, 0.2 mg/ml survival over 4 days
2 MR-M, 0.2 mg/ml survival over 4 days
3 CT-M, 0.2 mg/ml survival over 4 days
4 non-specific mouse IgG, 0.2 mg/ml survival over 4 days
5 control—PBS 10 μl/g bodyweight survival over 4 days

Clinical chemistry: Blood urea nitrogen (BUN) concentrations for renal function were measured baseline and day 1 after CLP. Blood samples were obtained from the cavernous sinus with a capillary under light ether anaesthesia. Measurements were performed by using an AU 400 Olympus Multianalyser. The 4-day mortality and the average BUN concentrations are given in table 4.

TABLE 4
4-day mortality and BUN concentrations

| | survival | BUN pre CLP | BUN day 1 |
|---|---|---|---|
| 4-day mortality | (%) | (mM) | (mM) |
| PBS | 0 | 8.0 | 23.2 |
| non-specific mouse IgG | 0 | 7.9 | 15.5 |
| CT-M | 10 | 7.8 | 13.5 |
| MR-M | 30 | 8.1 | 24.9 |
| NT-M | 70 | 8.8 | 8.2 |

It can be seen from Table 4 that the NT-M antibody reduced mortality considerably. After 4 days 70% of the mice survived when treated with NT-M antibody. When treated with MR-M antibody 30% of the animals survived and when treated with CT-M antibody 10% of the animals survived after 4 days. In contrast thereto all mice were dead after 4 days when treated with unspecific mouse IgG. The same result was obtained in the control group where PBS (phosphate buffered saline) was administered to mice. The blood urea nitrogen or BUN test is used to evaluate kidney function, to help diagnose kidney disease, and to monitor patients with acute or chronic kidney dysfunction or failure. The results of the S-BUN Test revealed that the NT-M antibody was the most effective to protect the kidney.

### b) Late Treatment of Sepsis

Animal model: 12-15 week-old male C57Bl/6 mice (Charles River Laboratories, Germany) were used for the study. Peritonitis had been surgically induced under light 80soflurane anesthesia. Incisions were made into the left upper quadrant of the peritoneal cavity (normal location of the cecum). The cecum was exposed and a tight ligature was placed around the cecum with sutures distal to the insertion of the small bowel. One puncture wound was made with a 24-gauge needle into the cecum and small amounts of cecal contents were expressed through the wound. The cecum was replaced into the peritoneal cavity and the laparotomy site was closed. Finally, animals were returned to their cages with free access to food and water. 500 μl saline were given s.c. as fluid replacement.

Application and dosage of the compound (NT-M FAB2): NT-M FAB2 was tested versus: vehicle and versus control compound treatment. Treatment was performed after full development of sepsis, 6 hours after CLP (late treatment). Each group contained 4 mice and were followed over a period of 4 days.

Group Treatment (10 μl/g bodyweight) dose/Follow-Up:
1 NT-M, FAB2 0.2 mg/ml survival over 4 days
2 control non-specific mouse IgG, 0.2 mg/ml survival over 4 days
3 vehicle:—PBS 10 μl/g bodyweight survival over 4 days

TABLE 5
4-day mortality

| 4 day mortality | survival (%) |
|---|---|
| PBS | 0 |
| Non-specific mouse IgG | 0 |
| NT-M FAB2 | 75 |

It can be seen from Table 5 that the NT-M FAB 2 antibody reduced mortality considerably. After 4 days 75% of the mice survived when treated with NT-M FAB 2 antibody. In contrast thereto all mice were dead after 4 days when treated with non-specific mouse IgG. The same result was obtained in the control group where PBS (phosphate buffered saline) was administered to mice.

### Example 5—Administration of NT-H in Healthy Humans

The study was conducted in healthy male subjects as a randomized, double-blind, placebo-controlled, study with single escalating doses of NT-H antibody administered as intravenous (i.v.) infusion in 3 sequential groups of 8 healthy male subjects each (1st group 0.5 mg/kg, 2nd group 2 mg/kg, 3rd group 8 mg/kg) of healthy male subjects (n=6 active, n=2 placebo for each group). The main inclusion criteria were written informed consent, age 18-35 years, agreement to use a reliable way of contraception and a BMI between 18 and 30 kg/m². Subjects received a single i.v. dose of NT-H antibody (0.5 mg/kg; 2 mg/kg; 8 mg/kg) or placebo by slow infusion over a 1-hour period in a research unit. The baseline ADM-values in the 4 groups did not differ. Median ADM values were 7.1 μg/mL in the placebo group, 6.8 μg/mL in the first treatment group (0.5 mg/kg), 5.5 μg/mL in second treatment group (2 mg/kg) and 7.1 μg/mL in the third treatment group (8 mg/mL). The results show, that ADM-values rapidly increased within the first 1.5 hours after administration of NT-H antibody in healthy human individuals, then reached a plateau and slowly declined (FIG. 6).

### Example 6—Methods for the Measurement of DPP3 Protein and DPP3 Activity

Generation of antibodies and determination DPP3 binding ability: Several murine antibodies were produced and screened by their ability of binding human DPP3 in a specific binding assay (see Table 6).

Peptides/conjugates for immunization: DPP3 peptides for immunization were synthesized, see Table 6, (JPT Technologies, Berlin, Germany) with an additional N-terminal cystein (if no cystein is present within the selected DPP3-sequence) residue for conjugation of the peptides to Bovine Serum Albumin (BSA). The peptides were covalently linked to BSA by using Sulfolink-coupling gel (Perbio-science, Bonn, Germany). The coupling procedure was performed according to the manual of Perbio. Recombinant GST-hDPP3 was produced by USBio (United States Biological, Salem, MA, USA).

Immunization of mice, immune cell fusion and screening: Balb/c mice were intraperitoneally (i.p.) injected with 84 μg GST-hDPP3 or 100 μg DPP3-peptide-BSA-conjugates at day 0 (emulsified in TiterMax Gold Adjuvant), 84 μg or 100 μg at day 14 (emulsified in complete Freund's adjuvant) and 42 μg or 50 μg at day 21 and 28 (in incomplete Freund's adjuvant). At day 49 the animal received an intravenous (i.v.) injection of 42 μg GST-hDPP3 or 50 μg DPP3-peptide-BSA-conjugates dissolved in saline. Three days later the mice were sacrificed and the immune cell fusion was performed.

Splenocytes from the immunized mice and cells of the myeloma cell line SP2/0 were fused with 1 ml 50% polyethylene glycol for 30 s at 37° C. After washing, the cells were seeded in 96-well cell culture plates. Hybrid clones were selected by growing in HAT medium [RPMI 1640 culture medium supplemented with 20% fetal calf serum and HAT-Supplement]. After one week, the HAT medium was replaced with HT Medium for three passages followed by returning to the normal cell culture medium.

The cell culture supernatants were primarily screened for recombinant DPP3 binding IgG antibodies two weeks after fusion. Therefore, recombinant GST-tagged hDPP3 (USBiologicals, Salem, USA) was immobilized in 96-well plates (100 ng/well) and incubated with 50 μl cell culture supernatant per well for 2 hours at room temperature. After washing of the plate, 50 μl/well POD-rabbit anti mouse IgG was added and incubated for 1 h at RT. After a next washing step, 50 μl of a chromogen solution (3.7 mM o-phenylen-diamine in citrate/hydrogen phosphate buffer, 0.012% H₂O₂) were added to each well, incubated for 15 minutes at RT and the chromogenic reaction stopped by the addition of 50 μl 4N sulfuric acid. Absorption was detected at 490 mm. The positive tested microcultures were transferred into 24-well plates for propagation. After retesting the selected cultures were cloned and re-cloned using the limiting-dilution technique and the isotypes were determined.

### Mouse Monoclonal Antibody Production

Antibodies raised against GST-tagged human DPP3 or DPP3-peptides were produced via standard antibody production methods (Marx et al. 1997) and purified via Protein A. The antibody purities were ≥90% based on SDS gel electrophoresis analysis.

Characterization of Antibodies—Binding to hDPP3 and/or Immunization Peptide

To analyze the capability of DPP3/immunization peptide binding by the different antibodies and antibody clones a binding assay was performed:

Solid phase: Recombinant GST-tagged hDPP3 (SEQ ID NO. 36) or a DPP3 peptide (immunization peptide, SEQ ID NO. 37) was immobilized onto a high binding microtiter plate surface (96-Well polystyrene microplates, Greiner Bio-One international AG, Austria, 1 μg/well in coupling buffer [50 mM Tris, 100 mM NaCl, pH7,8], 1 h at RT). After blocking with 5% bovine serum albumin, the microplates were vacuum dried.

Labelling procedure (tracer): 100 μg (100 μl) of the different antiDPP3 antibodies (detection antibody, 1 mg/ml in PBS, pH 7.4) were mixed with 10 μl acridinium NHS-ester (1 mg/ml in acetonitrile, InVent GmbH, Germany; EP 0 353 971) and incubated for 30 min at room temperature. Labelled antiDPP3 antibody was purified by gel-filtration HPLC on Shodex Protein 5 μm KW-803 (Showa Denko, Japan). The purified labelled antibody was diluted in assay buffer (50 mmol/l potassium phosphate, 100 mmol/1 NaCl, 10 mmol/l Na₂-EDTA, 5 g/l bovine serum albumin, 1 g/l murine IgG, 1 g/l bovine IgG, 50 μmol/1 amastatin, 100 μmol/1 leupeptin, pH 7.4). The final concentration was approx. 5-7*10⁶ relative light units (RLU) of labelled compound (approx. 20 ng labelled antibody) per 200 μl. acridinium ester chemiluminescence was measured by using a Centro LB 960 luminometer (Berthold Technologies GmbH & Co. KG).

hDPP3 binding assay: the plates were filled with 200 μl of labelled and diluted detection antibody (tracer) and incubated for 2-4 h at 2-8° C. Unbound tracer was removed by washing 4 times with 350 μl washing solution (20 mM PBS, pH 7.4, 0.1% Triton X-100). Well-bound chemiluminescence was measured by using the Centro LB 960 luminometer (Berthold Technologies GmbH & Co. KG).

Characterization of Antibodies—hDPP3-Inhibition Analysis

To analyze the capability of DPP3 inhibition by the different antibodies and antibody clones a DPP3 activity assay with known procedure (Jones et al., 1982) was performed. Recombinant GST-tagged hDPP3 was diluted in assay buffer (25 ng/ml GST-DPP3 in 50 mM Tris-HCl, pH7,5 and 100 μM ZnCl₂) and 200 μl of this solution incubated with 10 μg of the respective antibody at room temperature. After 1 hour of pre-incubation, fluorogenic substrate Arg-Arg-βNA (20 μl, 2 mM) was added to the solution and the generation of free βNA over time was monitored using the Twinkle LB 970 microplate fluorometer (Berthold Technologies GmbH & Co. KG) at 37° C. Fluorescence of βNA is detected by exciting at 340 nm and measuring emission at 410 nm. Slopes (in RFU/min) of increasing fluorescence of the different samples are calculated. The slope of GST-hDPP3 with buffer control is appointed as 100% activity. The inhibitory ability of a possible capture-binder is defined as the decrease of GST-hDPP3 activity by incubation with said capture-binder in percent.

The following table represents a selection of obtained antibodies and their binding rate in Relative Light Units (RLU) as well as their relative inhibitory ability (%; table 6). The monoclonal antibodies raised against the below depicted DPP3 regions, were selected by their ability to bind recombinant DPP3 and/or immunization peptide, as well as by their inhibitory potential.

All antibodies raised against the GST-tagged, full-length form of recombinant hDPP3 show a strong binding to immobilized GST-tagged hDPP3. Antibodies raised against the SEQ ID NO.: 37 peptide bind as well to GST-hDPP3. The SEQ ID NO.: 37 antibodies also strongly bind to the immunization peptide.

The development of a luminescence immunoassay for the quantification of DPP3 protein concentrations (DPP3-LIA) as well as an enzyme capture activity assay for the quantification of DPP3 activity (DPP3-ECA) have been described recently (Rehfeld et al. 2019. JALM 3(6): 943-953), which is incorporated here in its entirety by reference.

TABLE 6
list of antibodies raised against full-length or sequences of hDPP3 and their
ability to bind hDPP3 (SEQ ID NO.: 36) or immunization peptide (SEQ ID NO.: 37)
in RLU, as well as the maximum inhibition of recombinant GST-hDPP3.

| | | | | hDPP3 | immunization | Max. |
|---|---|---|---|---|---|---|
| Sequence | | hDPP3 | | binding | peptide | inhibition |
| number | Antigen/Immunogen | region | Clone | [RLU] | binding [RLU] | of hDPP3 |
| SEQ ID | GST tagged recombinant FL-hDPP3 | 1-737 | 2552 | 3.053.621 | 0 | 65% |
| NO.: 36 | | | 2553 | 3.777.985 | 0 | 35% |
| | | | 2554 | 1.733.815 | 0 | 30% |
| | | | 2555 | 3.805.363 | 0 | 25% |
| SEQ ID | CETVINPETGEQIQSWYRSGE | 474- | 1963 | 141.822 | 2.163.038 | 60% |
| NO.: 37 | | 493 | 1964 | 100.802 | 2.041.928 | 60% |
| | | | 1965 | 99.493 | 1.986.794 | 70% |
| | | | 1966 | 118.097 | 1.990.702 | 65% |
| | | | 1967 | 113.736 | 1.909.954 | 70% |
| | | | 1968 | 105.696 | 2.017.731 | 65% |
| | | | 1969 | 82.558 | 2.224.025 | 70% |

### Example 7—Development of Procizumab

Antibodies raised against SEQ ID No.: 37 were characterized in more detail (epitope mapping, binding affinities, specificity, inhibitory potential). Here the results for clone 1967 of SEQ ID No.: 37 (AK1967; “Procizumab”) are shown as an example.

### Determination of AK1967 Epitope on DPP3:

For epitope mapping of AK1967 a number of N- or C-terminally biotinylated peptides were synthesized (peptides & elephants GmbH, Hennigsdorf, Germany). These peptides include the sequence of the full immunization peptide (SEQ ID No. 37) or fragments thereof, with stepwise removal of one amino acid from either C- or N-terminus (see table 8 for a complete list of peptides).

High binding 96 well plates were coated with 2 g μg Avidin per well (Greiner Bio-One international AG, Austria) in coupling buffer (500 mM Tris-HCl, pH 7.8, 100 mM NaCl). Plates were then washed and filled with specific solutions of biotinylated peptides (10 ng/well; buffer—1×PBS with 0.5% BSA). Anti-DPP3 antibody AK1967 was labelled with a chemiluminescence label according to Example 6. The plates were filled with 200 μl of labelled and diluted detection antibody (tracer) and incubated for 4 h at room temperature. Unbound tracer was removed by washing 4 times with 350 μl washing solution (20 mM PBS, pH 7.4, 0.1% Triton X-100). Well-bound chemiluminescence was measured by using the Centro LB 960 luminometer (Berthold Technologies GmbH & Co. KG). Binding of AK1967 to the respective peptides is determined by evaluation of the relative light units (RLU). Any peptide that shows a significantly higher RLU signal than the unspecific binding of AK1967 is defined as AK1967 binder. The combinatorial analysis of binding and non-binding peptides reveals the specific DPP3 epitope of AK1967.

Determination of Binding Affinities using Octet:

The experiment was performed using Octet Red96 (ForteBio). AK1967 was captured on kinetic grade anti-humanFc (AHC) biosensors. The loaded biosensors were then dipped into a dilution series of recombinant GST-tagged human DPP3 (100, 33.3, 11.1, 3.7 nM). Association was observed for 120 seconds followed by 180 seconds of dissociation. The buffers used for the experiment are depicted in table 7. Kinetic analysis was performed using a 1:1 binding model and global fitting.

TABLE 7
Buffers used for Octet measurements

| Buffer | Composition |
|---|---|
| Assay Buffer | PBS with 0.1% BSA, 0.02% Tween-21 |
| Regeneration Buffer | 10 mM Glycine buffer (pH 1.7) |
| Neutralization Buffer | PBS with 0.1% BSA, 0.02% Tween-21 |

### Western Blot Analysis of Binding Specificity of AK1967:

Blood cells from human EDTA-blood were washed (3× in PBS), diluted in PBS and lysed by repeated freeze-thaw-cycles. The blood cell lysate had a total protein concentration of 250 g/ml, and a DPP3 concentration of 10 μg/ml. Dilutions of blood cell lysate (1:40, 1:80, 1:160 and 1:320) and of purified recombinant human His-DPP3 (31.25-500 ng/ml) were subjected to SDS-PAGE and Western Blot. The blots were incubated in 1.) blocking buffer (1×PBS-T with 5% skim milk powder), 2.) primary antibody solution (AK1967 1:2.000 in blocking buffer) and 3.) HRP labelled secondary antibody (goat anti mouse IgG, 1:1.000 in blocking buffer). Bound secondary antibody was detected using the Amersham ECL Western Blotting Detection Reagent and the Amersham Imager 600 UV (both from GE Healthcare).

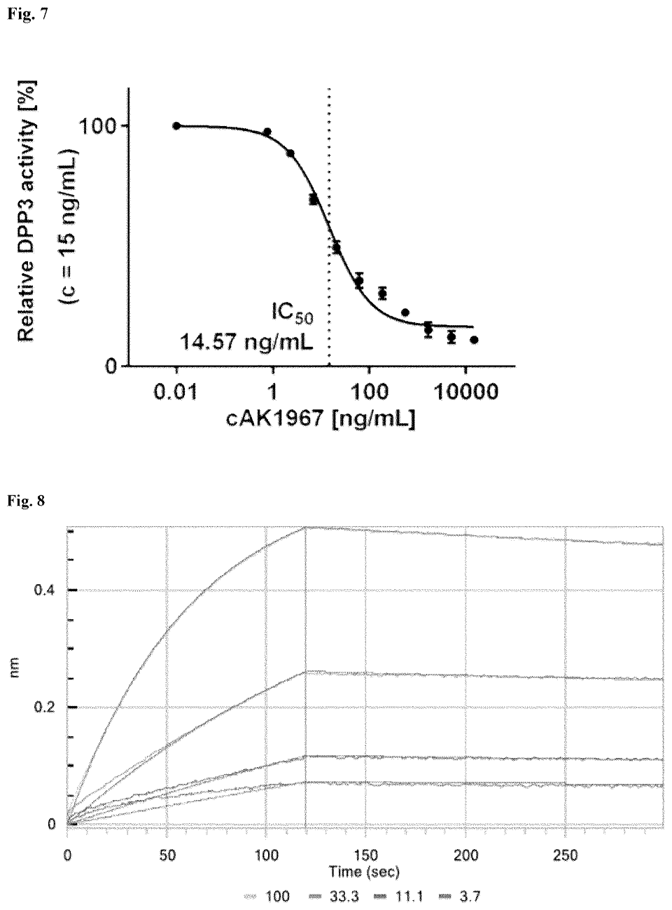

DPP3 inhibition assay: To analyze the capability of DPP3 inhibition by AK1967 a DPP3 activity assay with known procedure (Jones et al., 1982) was performed as described in example 6. The inhibitory ability AK1967 is defined as the decrease of GST-hDPP3 activity by incubation with said antibody in percent. The resulting lowered DPP3 activities are shown in an inhibition curve in FIG. 7.

Epitope mapping: The analysis of peptides that AK1967 binds to and does not bind to revealed the DPP3 sequence INPETG (SEQ ID No.: 38) as necessary epitope for AK1967 binding (see table 8).

Binding affinity: AK1967 binds with an affinity of 2.2*10-9 M to recombinant GST-hDPP3 (kinetic curves see FIG. 8).

### Specificity and Inhibitory Potential:

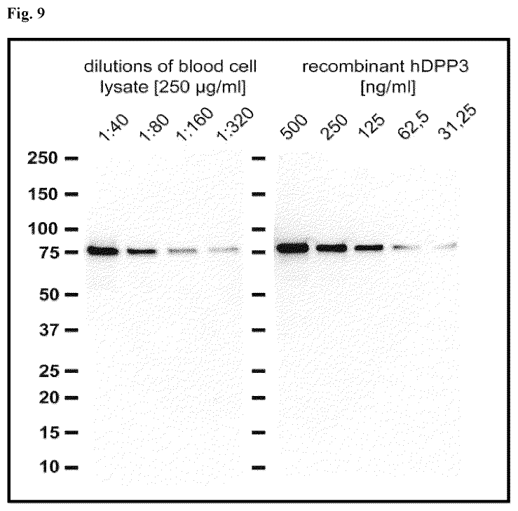

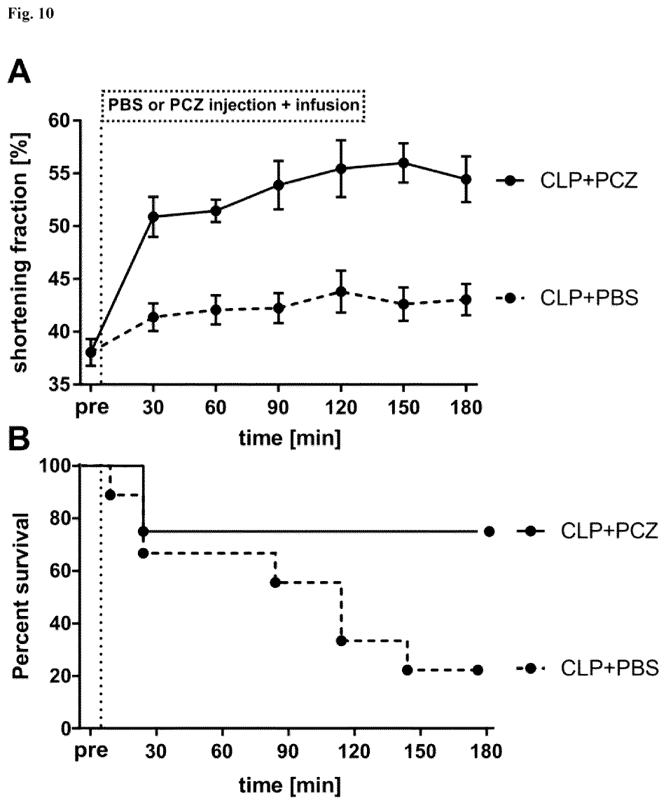

The only protein detected with AK1967 as primary antibody in lysate of blood cells was DPP3 at 80 kDa (FIG. 9). The total protein concentration of the lysate was 250 μg/ml whereas the estimated DPP3 concentration is about 10 gig/ml. Even though there is 25 times more unspecific protein in the lysate, AK1967 binds and detects specifically DPP3 and no other unspecific binding takes place.

AK1967 inhibits 15 ng/ml DPP3 in a specific DPP3 activity assay with an IC50 of about 15 ng/ml (FIG. 7).

### Chimerization/Humanization:

The monoclonal antibody AK1967 (“Procizumab”), with the ability of inhibiting DPP3 activity by 70%, was chosen as possible therapeutic antibody and was also used as template for chimerization and humanization.

### Humanization of Murine Antibodies May be Conducted According to the Following Procedure:

For humanization of an antibody of murine origin the antibody sequence is analyzed for the structural interaction of framework regions (FR) with the complementary determining regions (CDR) and the antigen. Based on structural modelling an appropriate FR of human origin is selected and the murine CDR sequences are transplanted into the human FR. Variations in the amino acid sequence of the CDRs or FRs may be introduced to regain structural interactions, which were abolished by the species switch for the FR sequences. This recovery of structural interactions may be achieved by random approach using phage display libraries or via directed approach guided by molecular modeling (Almagro and Fransson, 2008. Humanization of antibodies. Front Biosci. 13:1619-33).

With the above context, the variable region can be connected to any subclass of constant regions (IgG, IgM, IgE. IgA), or only scaffolds, Fab fragments, Fv, Fab and F(ab)2. The murine antibody variant contains an IgG2a backbone. For chimerization and humanization a human IgG1K backbone was used. For epitope binding only the Complementarity Determining Regions (CDRs) are of importance. The CDRs for the heavy chain and the light chain of the murine anti-DPP3 antibody (AK1967; “Procizumab”) are shown in SEQ ID No. 42, SEQ ID No. 43 and SEQ ID No. 44 for the heavy chain and SEQ ID No. 45, sequence KVS and SEQ ID No. 46 for the light chain, respectively.

Sequencing of the anti-DPP3 antibody (AK1967; “Procizumab”) revealed an antibody heavy chain variable region (H chain) according to SEQ ID No.: 47 and an antibody light chain variable region (L chain) according to SEQ ID No.: 48.

### Example 8—Effect of Procizumab in Sepsis-Induced Heart Failure

In this experiment, the effect of Procizumab injection in sepsis-induced heart failure rats (Rittirsch et al. 2009) was studied by monitoring the shortening fraction.

### CLP Model of Septic Shock:

Male Wistar rats (2-3 months, 300 to 400 g, group size refers to table 9) from the Centre d'élevage Janvier (France) were allocated randomly to one of three groups. All the animals were anesthetized using ketamine hydrochloride (90 mg/kg) and xylazine (9 mg/kg) intraperitoneally (i.p.). For induction of polymicrobial sepsis, cecal ligation and puncture (CLP) was performed using Rittirsch's protocol with minor modification. A ventral midline incision (1.5 cm) was made to allow exteriorization of the cecum. The cecum is then ligated just below the ileocecal valve and punctured once with an 18-gauge needle. The abdominal cavity is then closed in two layers, followed by fluid resuscitation (3 ml/100 g body of weight of saline injected subcutaneously) and returning the animal to its cage. Sham animals were subjected to surgery, without getting their cecum punctured. CLP animals were randomized between placebo and therapeutic antibody.

### Study Design:

The study flow is depicted in FIG. 8. After CLP or sham surgery the animals were allowed to rest for 20 hours with free access to water and food. Afterwards they were anesthetized, tracheotomy done and arterial and venous line laid. At 24 hours after CLP surgery either AK1967 or vehicle (saline) were administered with 5 mg/kg as a bolus injection followed by a 3 h infusion with 7.5 mg/kg. As a safety measure, hemodynamics were monitored invasively and continuously from t=0 till 3 h.

At t=0 (baseline) all CLP animals are in septic shock and developed a decrease in heart function (low blood pressure, low shortening fraction). At this time point Procizumab or vehicle (PBS) were injected (i.v.) and saline infusion was started. There were 1 control group and 2 CLP groups which are summarized in the table below (table 9). At the end of the experiment, the animals were euthanized, and organs harvested for subsequent analysis.

TABLE 9
list of experimental groups (sepsis-induced heart failure)

| Group | Number of Animals | CLP | Treatment |
|---|---|---|---|
| Sham | 7 | No | PBS |
| CLP-PBS | 6 | Yes | PBS |
| CLP-PCZ | 4 | Yes | PCZ |

### Invasive Blood Pressure:

Hemodynamic variables were obtained using the AcqKnowledge system (BIOPAC Systems, Inc., USA). It provides a fully automated blood pressure analysis system. The catheter is connected to the BIOPAC system through a pressure sensor.

For the procedure, rats were anesthetized (ketamine and xylazine). Animals were moved to the heating pad for the desired body temperature to 37-37.5° C. The temperature feedback probe was inserted into the rectum. The rats were placed on the operating table in a supine position. The trachea was opened and a catheter (16G) was inserted for an external ventilator without to damage carotid arteries and vagus nerves. The arterial catheter was inserted into the right carotid artery. The carotid artery is separate from vagus before ligation. A central venous catheter was inserted through the left jugular vein allowing administration of PCZ or PBS. Following surgery, the animals were allowed to rest for the stable condition prior to hemodynamic measurements. Then baseline blood pressure (BP) were recorded. During the data collection, saline infusion via arterial line was stopped.

### Echocardiography:

Animals were anesthetized using ketamine hydrochloride. Chests were shaved and rats were placed in decubitus position. For transthoracic echocardiographic (TTE) examination a commercial GE Healthcare Vivid 7 Ultra-sound System equipped with a high frequency (14-MHz) linear probe and 10-MHz cardiac probe was used. All examinations were recorded digitally and stored for subsequent off-line analysis.

Grey scale images were recorded at a depth of 2 cm. Two-dimensional examinations were initiated in a parasternal long axis view to measure the aortic annulus diameter and the pulmonary artery diameter. M-mode was also employed to measure left ventricular (LV) dimensions and assess fractional shortening (FS %). LVFS was calculated as LV end-diastolic diameter−LV end-systolic diameter/LV end-diastolic diameter and expressed in %. The time of end-diastole was therefore defined at the maximal diameter of the LV. Accordingly, end-systole was defined as the minimal diameter in the same heart cycle. All parameters were measured manually. Three heart cycles were averaged for each measurement.

From the same parasternal long axis view, pulmonary artery flow was recorded using pulsed wave Doppler. Velocity time integral of pulmonary artery outflow was measured. From an apical five-chamber view, mitral flow was recorded using pulsed Doppler at the level of the tip of the mitral valves.

### Results:

The sepsis-induced heart failure rats treated with PBS (CLP+PBS) show reduced shortening fraction compared to the sham animals (FIG. 10 A). The CLP+PBS group also displays high mortality rate (FIG. 10B). In contrast, application of Procizumab to sepsis-induced heart failure rats improves shortening fraction (FIG. 10A) and drastically reduces the mortality rate (FIG. 10R).

### Example 9—Effect of Procizumab on Heart and Kidney Function

The effect of Procizumab in isoproterenol-induced heart failure in mice was studied by monitoring the shortening fraction and renal resistive index.

### Isoproterenol-Induced Cardiac Stress in Mice:

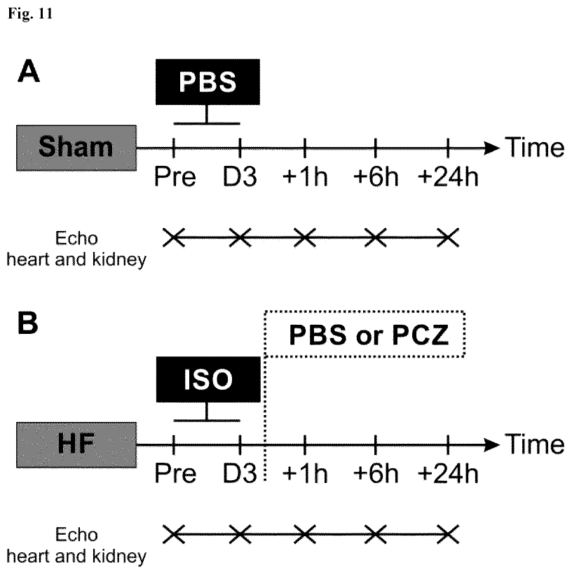

Acute heart failure was induced in male mice at 3 months of age by two daily subcutaneous injections of 300 mg/kg of Isoproterenol, a non-selective β-adrenergic agonist (DL-Isoproterenol hydrochloride, Sigma Chemical Co) (ISO) for two days (Vergaro et al, 2016). The ISO dilution was performed in NaCl 0.9%. Isoproterenol-treated mice were randomly assigned to two groups (Table 10) and PBS or Procizumab (10 mg/kg) were injected intravenously after baseline echocardiography (Gao et al., 2011) and renal resistive index measurements (Lubas et al., 2014, Dewitte et al, 2012) were performed at day 3 (FIGS. 11 A and B). Cardiac function was assessed by echocardiography (Gao et al., 2011) and by the renal resistive index (Lubas et al., 2014, Dewitte et al, 2012) at 1 hour, 6 hours and 24 hours (FIGS. 11 A and B). The group of mice that was injected with vehicle (PBS) instead of isoproterenol was subjected to no further pharmacological treatment and served as the control group (Table 10).

TABLE 10
list of experimental groups (isoproterenol-induced heart failure)

| Group | Number of Animals | Treatment |
|---|---|---|
| Sham + PBS | 27 | PBS |
| HF + PBS | 15 | PBS |
| HF + PCZ | 20 | PCZ |

### Results:

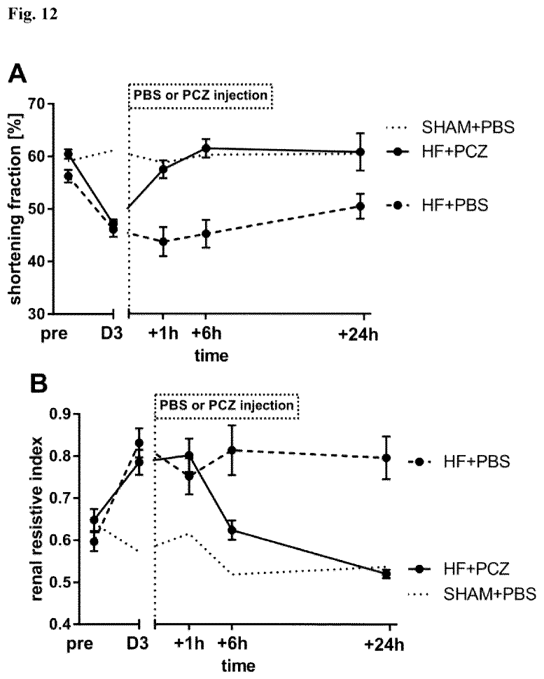

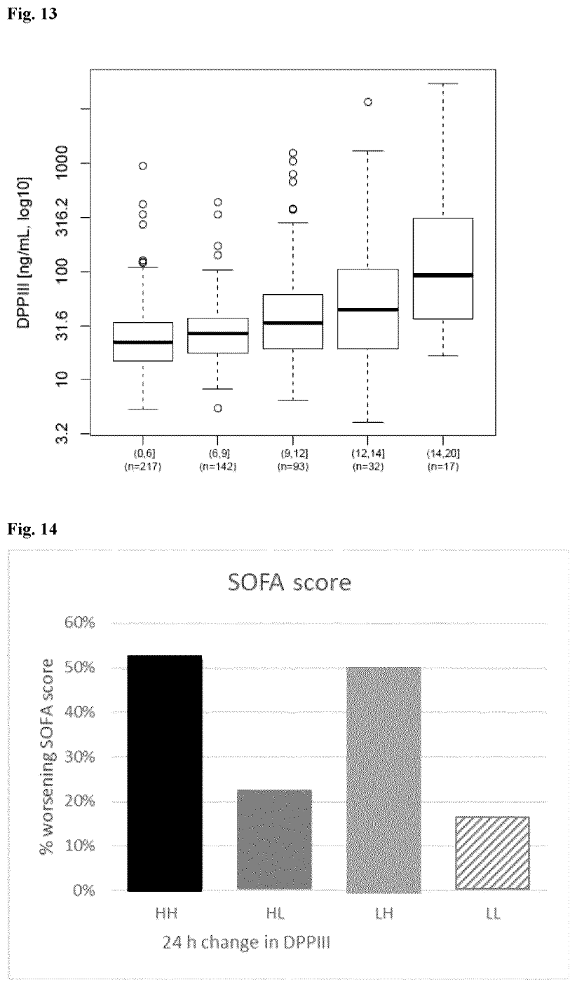

Application of Procizumab to isoproterenol-induced heart failure mice restores heart function within the first hour after administration (FIG. 12A). Kidney function of sick mice shows significant improvement at 6 hours post PCZ injection and is comparable to the kidney function of sham animals at 24 hours (FIG. 12B).

### Example 10—DPP3 and Organ Dysfunction in Sepsis

AdrenOSS-1 study is a prospective, multicentric observational study (ClinicalTrials.gov NCT02393781) in patients with severe sepsis and septic shock. Twenty-four centers in five European countries (France, Belgium, The Netherlands, Italy, and Germany) contributed to the trial achievement of 583 enrolled patients (recruited from June 2015 to May 2016). Of the 583 patients enrolled, 581 patients had DPP3 plasma levels measured. The study protocol was approved by the local ethics committees and was conducted in accordance with the Declaration of Helsinki. The study enrolled patients aged 18 years and older who were (1) admitted to the ICU for sepsis or septic shock or (2) transferred from another ICU in the state of sepsis and septic shock within less than 24 h after admission. Included patients were stratified by severe sepsis and septic shock based on definitions for sepsis and organ failure from 2001 (Levy et al. 2003. 2001 SCCM/ESICM/ACCP/ATS/SIS International Sepsis Definitions Conference. Crit Care Med. 31(4):1250-6). Patients were treated according to local practice, and treatments as well as procedures were registered. The primary endpoint was 28-day mortality. Secondary endpoints concerned organ failure (as defined by the Sequential Organ Failure Assessment [SOFA] score) and organ support, vasopressor/inotrope use, fluid balance, and use of renal replacement therapy (RRT).

Upon admission, demographics (age, sex), body mass index, presence of septic shock, type of ICU admission, organ dysfunction scores (SOFA, Acute Physiologic Assessment and Chronic Health Evaluation II [APACHE II]), origin of sepsis, pre-existing comorbidities (i.e., treated within the last year), past medical history, laboratory values, and organ support were recorded, and blood was drawn for measurement of bio-ADM and other markers. After patient enrolment, the following data were collected daily during the first week: SOFA score, antimicrobial therapies, fluid balance, ventilation status, Glasgow Coma Scale score, central venous pressure, need for RRT, invasive procedures for sepsis control, and vasopressor/inotrope treatment. Moreover, discharge status and mortality were recorded on day 28 after ICU admission. Blood for the central laboratory was sampled within 24 h after ICU admission and on day 2 (mean 47 h, SD 9 h) after the first sample. Samples were subsequently processed and stored at −80° C.

DPP3 measurement: An immunoassay (LIA) or an enzyme activity assays (ECA) detecting the amount of human DPP3 (LIA) or the activity of human DPP3 (ECA), respectively, was used for determining the DPP3 level in patient plasma. Antibody immobilization, labelling and incubation were performed as described in Rehfeld et al. (Rehfeld et al. 2019. JALM 3(6): 943-953).

The AdrenOSS-1 study was used to assess the association between circulating DPP3, organ dysfunction (e.g. cardiovascular and renal dysfunction) in patients admitted for sepsis and septic shock. Median DPP3 measured at admission in all AdrenoSS-1 patients was 45.1 ng/mL (inter quartile range 27.5-68.6). High DPP3 levels measured at admission were associated with worse metabolic parameters, renal and cardiac function and SOFA score: patients with DPP3 levels below the median had a median SOFA score (points) of 6 (IQR 4-9) compared to a median SOFA score of 8 (IQR 5-11) for patients with DPP3 levels above the median of 45.1 ng/mL (FIG. 13)

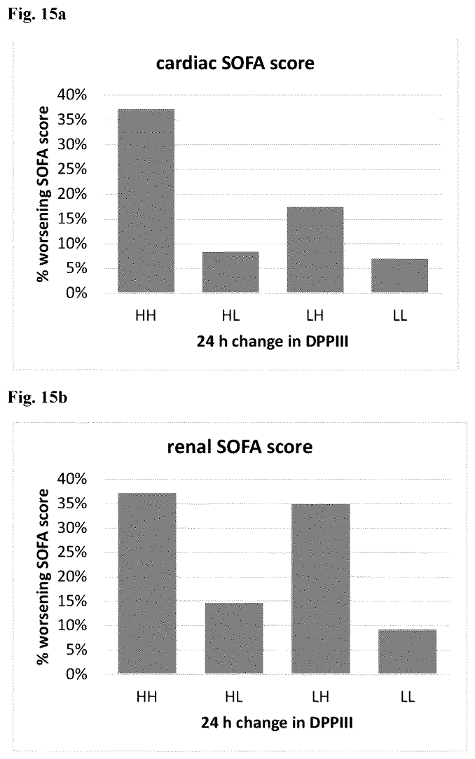

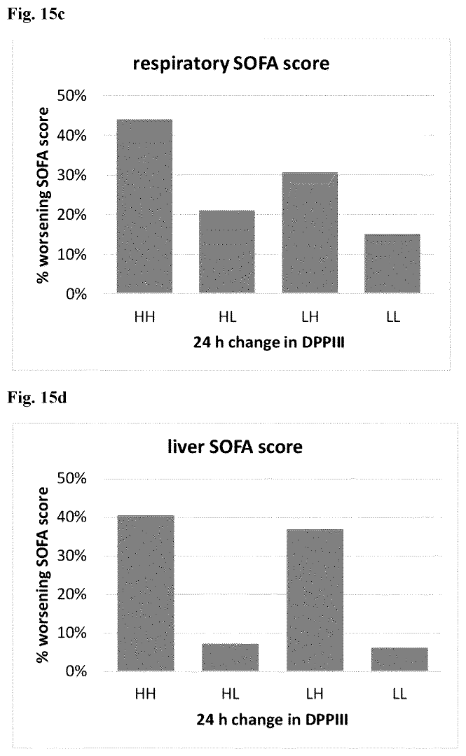

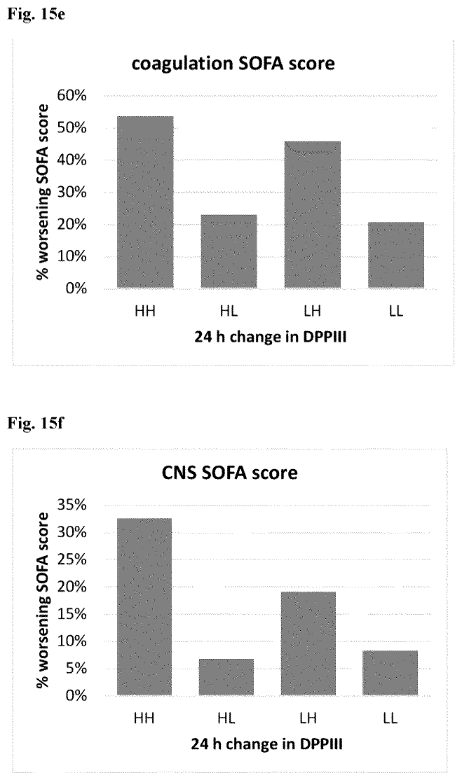

Whatever levels of DPP3 at admission, high concentrations of DPP3 levels 24 hours later were associated with worst SOFA scores whether global FIG. 14 or by organ (FIG. 15 A-F).

In summary these data showed that high levels of DPP3 were associated with survival and the extent of organ dysfunction in a large international cohort septic or septic shock patients. The study found marked association between DPP3<45.1 ng/ml at admission and short-term survival as well as the prognostic cut-off value of 45.1 μg/ml in both sepsis and septic shock. Concerning organ dysfunction, there was a positive relationship between DPP3 and SOFA score at ICU admission. More importantly, the relationship between DPP3 levels and extent of organ dysfunction, seen at ICU admission, was also true during the recovery phase. Indeed, patients with high DPP3 levels at admission who showed a decline towards normal DPP3 values at day 2 were more likely to recover all organ function including cardiovascular, kidney, lung, liver.

### Example 11—DPP3 in Septic and Cardiogenic Shock

DPP3 concentration in plasma of patients with sepsis/septic shock and cardiogenic shock was determined and related to the short term-mortality of the patients.

### a) Study Cohort—Sepsis/Septic Shock

The same study as in example 10 was analyzed (AdrenOSS-1). In this study 292 patients out of 583 patients were diagnosed with septic shock.

### b) Study Cohort—Cardiogenic Shock

Plasma samples from 108 patients that were diagnosed with cardiogenic shock were screened for DPP3. Blood was drawn within 6 h from detection of cardiogenic shock. Mortality was followed for 7 days.

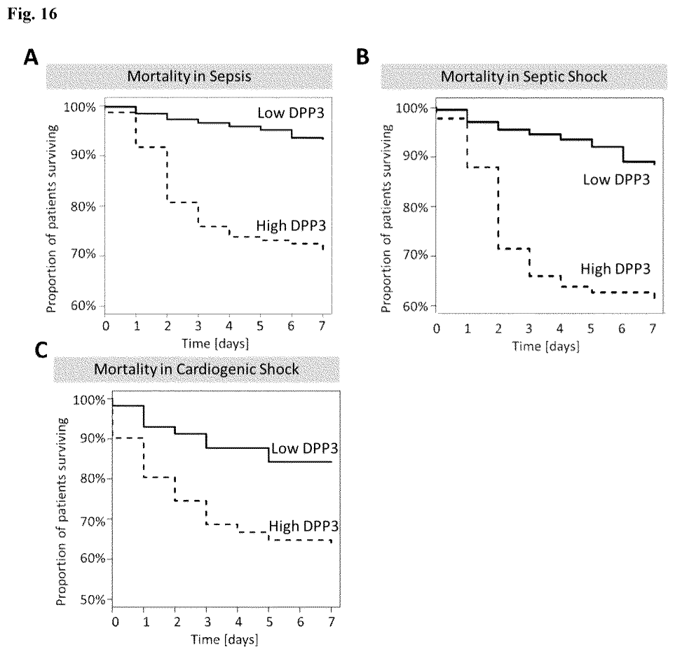

Results: Short-term patients' survival in sepsis patients was related to the DPP3 plasma concentration at admission. Patients with DPP3 plasma concentration above 40.5 ng/mL (3rd Quartile) had an increased mortality risk compared to patients with DPP3 plasma concentrations below this threshold (FIG. 16A). Applying this cut-off to the sub-cohort of septic shock patients, revealed an even more pronounced risk for short-term mortality in relation to high DPP3 plasma concentrations (FIG. 16B). When the same cut-off is applied to patients with cardiogenic shock, also an increased risk for short-term mortality within 7 days is observed in patients with high DPP3 (FIG. 16C).

### Example 12—Prediction of DPP3 Increase in Sepsis and Septic Shock (AdrenOSS-1)

The AdrenOSS-1 study as described in example 10 was used to analyze whether DPP3 levels at baseline may be able to predict an increase in DPP3 on the following days.

### Results:

The DPP3 plasma levels in septic shock patients (n=292) at baseline (day 1, DPP3.d1) were statistically analysed with the aim of determining a threshold to predict an increase in DPP3 plasma concentration above 50 ng/ml on the following days. The DPP3 concentration of 50 ng/ml reflects a threshold above which patients a) are beyond the DPP3 upper limit of normal, b) have a high organ dysfunction and mortality rate (see example 11; Blet et al. 2021. Crit Care 25(1): 61) and c) have been shown to have a lower treatment effect for the N-terminal ADM-antibody Adrecizumab (WO2021/170838).

TABLE 11
DPP3 plasma levels in septic shock patients

| DPP3 concentration thresholds | DPP3.d1 <50 | DPP3.d1 <40 | DPP3.d1 <30 | DPP3.d1 <22 |
|---|---|---|---|---|
| at baseline (d1) | ng/ml | ng/ml | ng/ml | ng/ml |
| DPP3 below 50 ng/ml during | 201 | 187 | 149 | 104 |
| ICU stay | | | | |
| rising DPP3 above 50 ng/ml | 22 | 14 | 7 | 1 |
| during ICU stay | | | | |
| Total number of patients | 223 | 201 | 156 | 105 |
| % of patients with rising DPP3 | 10% | 7% | 4% | 1% |
| above 50 ng/ml during ICU stay | | | | |
| on days 2 and 3 | | | | |

Different DPP3 threshold values at baseline (d1) were analysed for their ability to predict the percentage of patients with DPP3 plasma concentration increase above 50 ng/ml in the following days (day 2 and day 3). Table 11 shows that the lower the DPP3 plasma concentration at baseline (DPP3.d1), the lower the percentage of patients with an increase in DPP3 above 50 ng/ml in the following days. In this septic shock population, 223 and 156 patients have a DPP3 concentration below <50 or <30 ng/ml at baseline, respectively. For 156 patients below 30 ng/ml at baseline, 7 (4%) patients show a DPP3 concentration rise above the DPP3 threshold of 50 ng/ml in the following days. On the other hand, among the 67 septic shock patients with DPP3 plasma concentrations between 30 and 50 ng/ml at baseline, 15 (22.4%) patients increased their DPP3 plasma levels above 50 ng/ml in the following days. As a consequence, a low DPP3 threshold (in the range between 22 ng/ml and 40 ng/ml) is suitable predict a later increase in DPP3 which may be used for a treatment decision at baseline for, e.g., the use of an anti-ADM antibody (Adrecizumab) therapy in patients with septic shock. The low DPP3 concentration threshold at baseline (d1) ensures, that the DPP3 pathological pathway (which is associated with high short-term organ dysfunction and mortality) is not the predominant pathway in the selected septic shock population. Therefore, this septic shock population with DPP3 concentrations below the above-mentioned threshold ranges at baseline may have a higher treatment effect from, e.g., anti-ADM antibody therapy (Adrecizumab).

In a second step, the septic shock population with bio-ADM plasma concentrations above 70 μg/ml were anakyzed. Bio-ADM concentrations above 70 μg/ml have been associated with sepsis severity, development of organ dysfunction, including vasopressor/inotrope dependency (Marino et al. 2014. Critical Care 18: R34; Caironi et al. 2017. Chest 152(2):312-320; Mebazaa et al. 2018. Crit Care 22: 354). Different DPP3 threshold values at baseline (d1) were analysed for their ability to predict the percentage of patients with DPP3 plasma concentration increase above 50 ng/ml in the following days. Table 12 again shows that the lower the DPP3 plasma concentration at baseline (DPP3.d1), the lower the percentage of septic shock patients with high bio-ADM with an increase in DPP3 above 50 ng/ml in the following days. In this septic shock and high bio-ADM population, 154 and 100 patients have a DPP3 concentration below <50 or <30 ng/ml at baseline, respectively. For 100 patients below 30 ng/ml at baseline, 4 (4%) patients show a DPP3 concentration rise above the DPP3 threshold of 50 ng/ml in the following 2 days. On the other hand, among the 54 septic shock patients with DPP3 plasma concentrations between 30 and 50 ng/ml at baseline, 13 (24.1%) patients increased their DPP3 plasma levels above 50 ng/ml in the following days.

TABLE 12
DPP3 plasma levels in septic shock patients with a bio-ADM level above 70 pg/ml

| DPP3 concentration cut offs | DPP3.d1 <50 | DPP3.d1 <40 | DPP3.d1 <30 | DPP3.d1 <22 |
|---|---|---|---|---|
| at baseline (d1) | ng/ml | ng/ml | ng/ml | ng/ml |
| DPP3 below 50 ng/ml during | 137 | 125 | 96 | 61 |
| ICU stay | | | | |
| rising DPP3 above 50 ng/ml | 17 | 10 | 4 | 0 |
| during ICU stay | | | | |
| Total number of patients | 154 | 135 | 100 | 61 |
| % of patients with rising DPP3 | 11% | 7% | 4% | 0% |
| above 50 ng/ml during ICU | | | | |
| stay on days 2 and 3 | | | | |

### Example 13—NT-ADM Antibodies in Patients with Septic Shock (AdrenOSS-2)

AdrenOSS-2 is a double-blind, placebo-controlled, randomized, multicenter, proof of concept and dose-finding phase II clinical trial to investigate the safety, tolerability and efficacy of the N-terminal ADM antibody named Adrecizumab in patients with septic shock and elevated adrenomedullin (Geven et al. BMJ Open 2019; 9:e024475). In total, 301 patients with septic shock and bio-ADM concentration >70 μg/mL were randomized (2:1:1) to treatment with a single intravenous infusion over approximately 1 hour with either placebo (n=152), adrecizumab 2 ng/kg (n=72) or adrecizumab 4 ng/kg (n=77). All-cause mortality within 28 (90) days after inclusion was 25.8% (34.8%). Mean age was 68.4 years and 61% were male. For the per protocol analysis, n=294 patients remained eligible, and 14-day all-cause mortality rate was 18.5%.

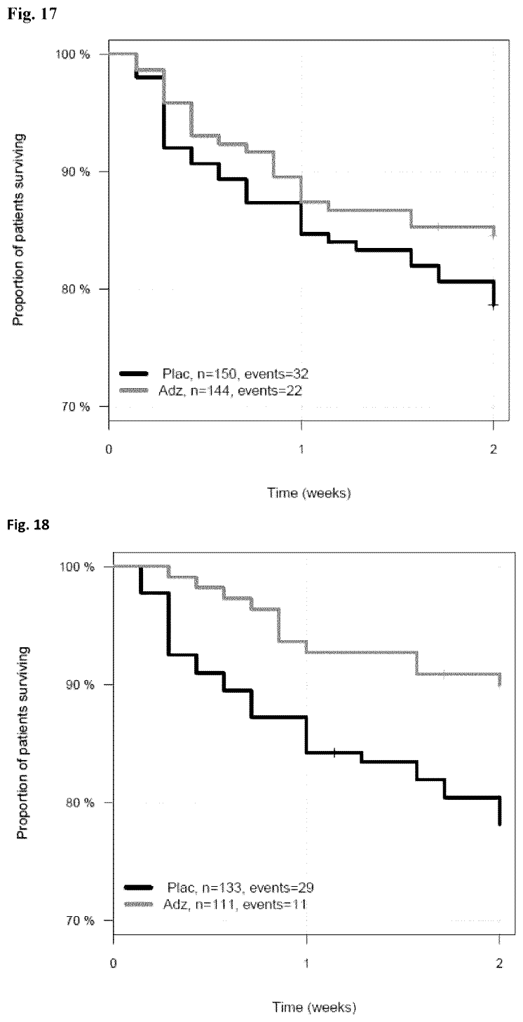

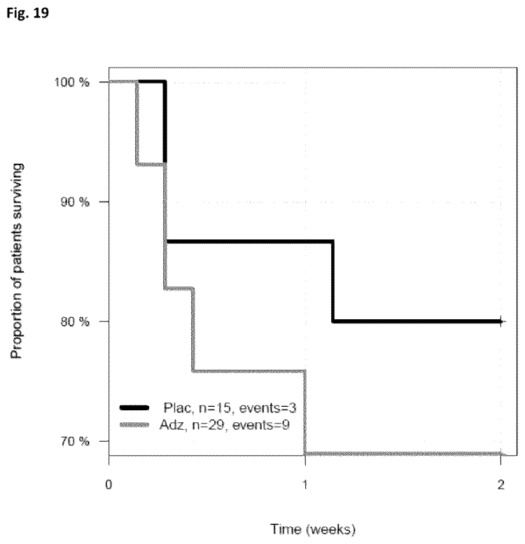

In patients treated with Adrecizumab (both doses combined, per protocol population), a trend to lower short-term mortality (14 days post admission) was observed compared to placebo (Hazard ratio (HR) 0.701 [0.408-1.21], p=0.100) (FIG. 17). Surprisingly, in patients with a DPP3 concentration on admission below 50 ng/mL, the treatment effect was more pronounced (n=244, HR 0.426, p=0.007) (FIG. 18), while in patients with an elevated DPP3 (above 50 ng/mL, n=44), outcome was comparable between Adrecizumab and placebo (HR 1.69, p=0.209) (FIG. 19).

Treatment effects for different DPP3 thresholds (14-day mortality) are summarized in table 13.

TABLE 13
Hazard risks (HR) for 14-day mortality
with different DPP3 concentrations

| | | | p-value |
|---|---|---|---|
| | n | HR | (1-sided, log rank) |
| all | 294 | 0.701 | 0.100 |
| DPP3 <70 | 261 | 0.546 | 0.027 |
| DPP3 >70 | 27 | 1.24 | 0.384 |
| DPP3 <60 | 254 | 0.449 | 0.008 |
| DPP3 >60 | 34 | 1.62 | 0.231 |
| DPP3 <50 | 244 | 0.426 | 0.007 |
| DPP3 >50 | 44 | 1.69 | 0.209 |
| DPP3 <40 | 227 | 0.385 | 0.005 |
| DPP3 >40 | 61 | 1.35 | 0.286 |

### Example 14—Lower DPP3 Thresholds in AdrenOSS-2 and Efficacy of NT-ADM Antibody Therapy

To validate the findings from example 9 and for proof of concept, the different lower thresholds for DPP3 at baseline (day 1) were assessed for efficacy of the anti-ADM antibody (Adrecizumab) therapy in the septic shock population with high bio-ADM from the AdrenOSS-2 study cohort and patients with a DPP3 plasma value above the different lower thresholds were excluded from the analyses. Efficacy of the anti-ADM antibody therapy was specifically assessed to what concerns the mortality endpoint in the placebo and treated arms.

### Results:

The DPP3 plasma levels in septic shock with high bio-ADM (above 70 μg/ml) patients (n=298) at baseline and in the following 144 h were statistically analysed with the aim of determining a DPP3 threshold at baseline to assess anti-ADM antibody therapy efficacy. Patients above the respective DPP3 plasma thresholds were excluded from the analyses. Different DPP3 plasma concentration thresholds have been applied for 28-day all-cause mortality evaluation using Kaplan-Meier plots comparing anti-ADM antibody therapy vs. placebo. The log-rank test was chosen for showing differences in mortality rates among treatment groups. Hazard ratios (HR) were calculated for each DPP3 plasma concentration threshold to estimate the reduction in mortality risk imposed by anti-ADM antibody therapy relative to placebo.

The DPP3 plasma concentration thresholds at baseline used were 50 ng/ml, 40 ng/ml, 30 ng/ml and 22 ng/ml, respectively. For each threshold, the number of patients excluded from all cause mortality analysis was determined. For the 50, 40, 30 and 22 ng/ml thresholds, 16%, 24%, 35% and 51% of patients were excluded from the analyses, respectively.

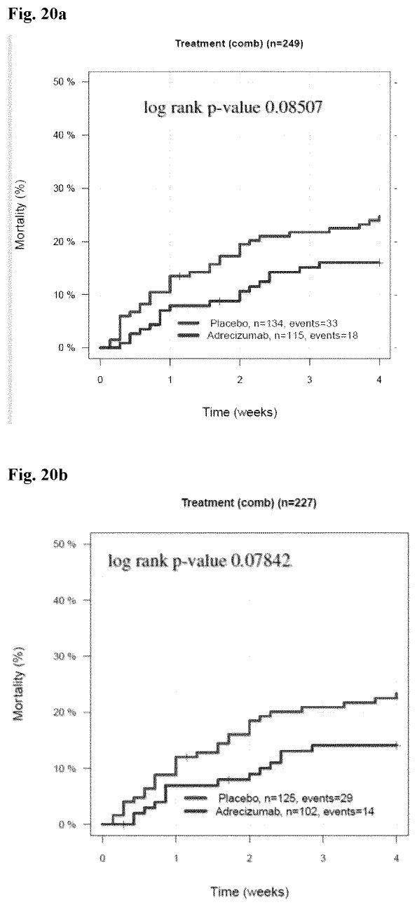

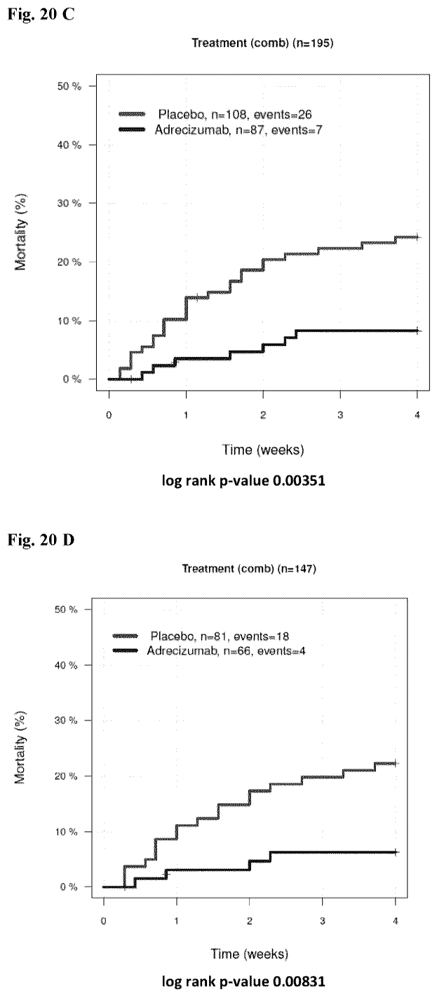

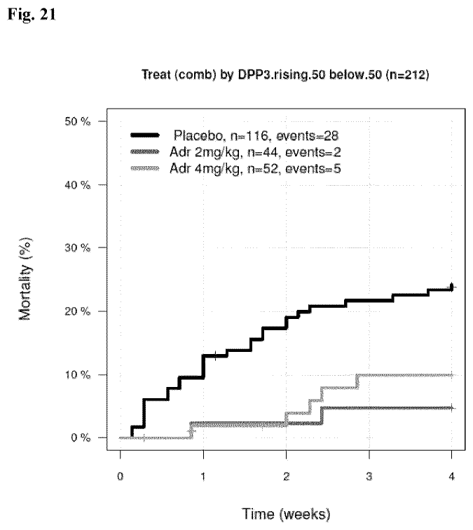

The HRs for each DPP3 plasma concentration threshold at baseline to estimate the reduction in mortality by the anti-ADM antibody therapy relative to placebo were also determined. For the 50, 40, 30 and 22 ng/ml thresholds, HRs were 0.606, 0.568, 0.309 and 0.258, respectively. This analysis shows that the lower the DPP3 plasma concentration threshold, the higher the reduction in mortality in the treated group. Similarly, all-cause mortality analysis using Kaplan-Meier indicate that the lower the DPP3 plasma concentration threshold, the more pronounced and significant is the reduction in mortality in the treated arm (FIG. 20 A-D).

The percentage of patients that show an increase in DPP3 plasma concentration above 50 ng/ml in the following 144 h was also estimated for each DPP3 plasma concentration threshold at baseline. Similar to the results from the AdrenOSS-1 study in example 12 it was shown that: the lower the DPP3 plasma concentration threshold at baseline, the lower the percentage of patients that showed an increase in their DPP3 plasma levels above the 50 ng/ml threshold in the following days. In this septic shock and high bio-ADM population, 249 and 195 patients have a DPP3 concentration below <50 or <30 ng/ml at baseline, respectively. For 195 patients below 30 ng/ml at baseline, 16 (8%) patients show a DPP3 concentration rise above the DPP3 threshold of 50 ng/ml in the following 6 days. On the other hand, among the 54 septic shock patients with DPP3 plasma concentrations between 30 and 50 ng/ml at baseline, 11 (20.4%) patients increased their DPP3 plasma levels above 50 ng/ml in the following days. These results show, that a lower DPP3 threshold (well below 50 ng/ml) is suitable to guide the use of and select the patients prone to benefit from anti-ADM antibody therapy.

The different thresholds for DPP3 levels at baseline (day 1) were further used for subgroup analyses for 28-day all-cause mortality evaluation using Kaplan-Meier plots in the treated and placebo arms comparing anti-ADM antibody therapy (Adrecizumab) vs. placebo. The DPP3 plasma concentration thresholds at baseline used were 50 ng/ml and 30 ng/ml.

When assessing all-cause mortality in the septic shock population with DPP3 levels below the threshold of 30 ng/ml (n=195), the mortality in the anti-ADM antibody (Adrecizumab) therapy arm was surprisingly significantly reduced compared to the placebo arm (FIG. 21 C). The same results are observed when including only patients with DPP3 values below 50 ng/ml, the mortality in the treated arm is lower than the placebo arm. Finally, when assessing all-cause mortality from admission to 28 days in septic shock patients with DPP3 levels below 50 ng/ml at baseline and continuously low (<50 ng/ml) DPP3 levels during the next 144 h, the mortality rate in the treated arm is significantly lower than in the placebo arm (FIG. 2l). As a consequence, a low DPP3 threshold (well below 50 ng/ml) at baseline is most suitable to select patients that will most benefit from the anti-ADM antibody therapy. In summary, patients with DPP3 levels above the threshold of 30 ng/ml at baseline have a higher chance to show an increase in DPP3 plasma concentration in the following days. The following increase in DPP3 plasma concentration above 50 ng/ml is associated with a lower efficacy of anti-ADM antibody therapy. Therefore, to stratify patients for anti-ADM antibody therapy, lower thresholds, well below 50 ng/ml, preferably in the range between 22 and 40 ng/ml, most preferred a threshold of 30 ng/ml should be used.

1. Method for the prediction of an increase of dipeptidyl peptidase 3 (DPP3) in a critical ill patient, the method comprising:
determining the level of DPP3 in a sample of bodily fluid of said patient,
comparing said determined level of DPP3 to a pre-determined threshold, wherein said threshold is in the range between 40 ng/ml and 22 ng/ml
wherein a level of DPP3 in said sample above said pre-determined threshold is indicative for an increase of DPP3 in said patient.

2. Method for the prediction of an increase of DPP3 in a critical ill patient according to claim 1, wherein said pre-determined threshold is between 30 ng/ml and 22 ng/ml.

3. Method for the prediction of an increase of DPP3 in a critical ill patient according to claim 1 or 2, wherein said pre-determined threshold is between 25 ng/ml and 22 ng/ml.

4. Method for the prediction of an increase of DPP3 in a critical ill patient according to any of claims 1-3, wherein said predicted increase is an increase to DPP3 levels equal to or above 40, preferred equal to or above 50 ng/ml.

5. Method for the prediction of an increase of DPP3 in a critically ill patient according to claims 1-4, wherein said predicted increase of the DPP3 level is equal to or above 10%, more preferred equal to or above 20%, even more preferred equal to or above 40%, even more preferred equal to or above 50%, even more preferred equal to or above 75%, even more preferred equal to or above 100%.

6. Method for the prediction of an increase of DPP3 in a critically ill patient according to claims 1-5, wherein said increase of DPP3 is within up to 12 hours, preferably up to 24, 48, 72, 96 hours, more preferred up to 5 days, even more preferred up to 6 days, most preferred up to 7 days.

7. Method for the prediction of an increase of DPP3 in a critical ill patient according to claim 1-6, wherein said patient is a patient with severe infection, sepsis, heart failure, chronic heart failure, acute heart failure (AHF), myocardial infarction (MI), stroke, a patient with organ dysfunction or organ failure (e.g., dysfunction or failure of liver, kidney, lung), a patient undergoing major surgery, a patient with trauma (e.g. burn trauma, polytrauma), a patient with shock and/or a patient running into shock, or alternatively ARDS.

8. Method for the prediction of an increase of DPP3 in a critical ill patient according to claim 7, wherein said shock is selected from the group comprising shock due to hypovolemia, cardiogenic shock, obstructive shock and distributive shock.

9. Method for the prediction of an increase of DPP3 in a critical ill patient according to claim 8, wherein
in case of cardiogenic shock said patient may have suffered an acute coronary syndrome (e.g., acute myocardial infarction) or wherein said patient has heart failure (e.g., acute decompensated heart failure), myocarditis, arrhythmia, cardiomyopathy, valvular heart disease, aortic dissection with acute aortic stenosis, traumatic chordal rupture or massive pulmonary embolism, or
in case of hypovolemic shock said patient may have suffered a hemorrhagic disease including gastrointestinal bleed, trauma, vascular etiologies (e.g. ruptured abdominal aortic aneurysm, tumor eroding into a major blood vessel) and spontaneous bleeding in the setting of anticoagulant use or a non-hemorrhagic disease including vomiting, diarrhea, renal loss, skin losses/insensible losses (e.g., burns, heat stroke) or third-space loss in the setting of pancreatitis, cirrhosis, intestinal obstruction, or
in case of obstructive shock said patient may have suffered a cardiac tamponade, tension pneumothorax, pulmonary embolism or aortic stenosis, or
in case of distributive shock said patient may have septic shock, neurogenic shock, anaphylactic shock or shock due to adrenal crisis.

10. Method for the prediction of an increase of DPP3 in a critical ill patient according to any of claims 7-9, wherein said shock is selected from the group comprising cardiogenic shock or septic shock.

11. Method for the prediction of an increase of DPP3 in a critical ill patient according to any of claims 1-10, wherein a patient is selected for therapy/treatment if the level of a DPP3 in said sample is below said pre-determined threshold, wherein said therapy is selected from the group of alkaline phosphatase, immune suppressors, corticosteroids, vasopressors, fluids, anti-Adrenomedullin antibodies or antibody fragments or scaffolds.

12. Method for the prediction of an increase of DPP3 in a critical ill patient according to claim 11, wherein said anti-adrenomedullin antibodies or anti-adrenomedullin antibody fragments or anti-adrenomedullin scaffolds are directed to the N-terminal part (amino acids 1-21) of adrenomedullin (ADM): YRQSMNNFQGLRSFGCRFGTC (SEQ ID No. 14)

13. Method for the prediction of an increase of DPP3 in a critical ill patient according to any of claims 1-12, wherein a patient is selected for therapy/treatment with DPP3 inhibitors if the level of a DPP3 in said sample is above said pre-determined threshold, wherein said DPP3 inhibitor is selected from the group of anti-DPP3-antibodies or anti-DPP3-antibody fragments or anti-DPP3 scaffolds.

14. Method for the prediction of an increase of DPP3 in a critical ill patient according to any of claims 1-13, wherein said level of DPP3 is either the amount of DPP3 protein and/or the level of active DPP3.

15. Method for the prediction of an increase of DPP3 in a critical ill patient according to any of claims 1-14, wherein said level of DPP3 is determined by different methods, comprising an immunoassay, an activity assay or mass spectrometric methods.

16. Method for the prediction of an increase of DPP3 in a critical ill patient according to any of claim 15, wherein said immunoassay is a sandwich immunoassay.

17. Method for the prediction of an increase of DPP3 in a critical ill patient according to any of claims 1-16, wherein said bodily fluid is selected from whole blood, serum or plasma.

18. A method for the prevention of a DPP3 increase in a critical ill patient the method comprising:
determining the level of DPP3 in a sample of bodily fluid of said patient,
comparing said determined level of DPP3 to a pre-determined threshold, wherein said pre-determined threshold is between 40 ng/ml and 22 ng/ml and wherein a level of a DPP3 in said sample above said pre-determined is indicative for an increase of DPP3 in said patient, and
administering a DPP3 inhibitor if said determined level of DPP3 is above said pre-determined threshold,
wherein said DPP3 inhibitor is an anti-DPP3-antibody and/or and anti-DPP3-antibody fragment and/or anti-DPP3 scaffold.

19. A method for the prevention of a DPP3 increase in a critical ill patient according to claim 18, wherein said pre-determined threshold is between 30 ng/ml and 22 ng/ml.

20. A method for the prevention of a DPP3 increase in a critical ill patient according to claim 18 or 19, wherein said pre-determined threshold is between 25 ng/ml and 22 ng/ml.

21. A method for the prevention of a DPP3 increase in a critical ill patient according to any of claims 18-20, wherein said increase is an increase to DPP3 levels equal to or above 40, preferred equal to or above 50 ng/ml.

22. A method for the prevention of a DPP3 increase in a critical ill patient according to claim 18-21, wherein said patient is a patient with severe infection, sepsis, heart failure, chronic heart failure, acute heart failure (AHF), myocardial infarction (MI), stroke, a patient with organ dysfunction or organ failure (e.g., dysfunction or failure of liver, kidney, lung), a patient undergoing major surgery, a patient with trauma (e.g. burn trauma, polytrauma), a patient with shock and/or a patient running into shock, or alternatively ARDS.

23. A method for the prevention of a DPP3 increase in a critical ill patient according to claim 22, wherein said shock is selected from the group comprising shock due to hypovolemia, cardiogenic shock, obstructive shock and distributive shock.

24. A method for the prevention of a DPP3 increase in a critical ill patient according to claim 23, wherein
in case of cardiogenic shock said patient may have suffered an acute coronary syndrome (e.g., acute myocardial infarction) or wherein said patient has heart failure (e.g., acute decompensated heart failure), myocarditis, arrhythmia, cardiomyopathy, valvular heart disease, aortic dissection with acute aortic stenosis, traumatic chordal rupture or massive pulmonary embolism, or
in case of hypovolemic shock said patient may have suffered a hemorrhagic disease including gastrointestinal bleed, trauma, vascular etiologies (e.g. ruptured abdominal aortic aneurysm, tumor eroding into a major blood vessel) and spontaneous bleeding in the setting of anticoagulant use or a non-hemorrhagic disease including vomiting, diarrhea, renal loss, skin losses/insensible losses (e.g., burns, heat stroke) or third-space loss in the setting of pancreatitis, cirrhosis, intestinal obstruction, or
in case of obstructive shock said patient may have suffered a cardiac tamponade, tension pneumothorax, pulmonary embolism or aortic stenosis, or
in case of distributive shock said patient may have septic shock, neurogenic shock, anaphylactic shock or shock due to adrenal crisis.

25. A method for the prevention of a DPP3 increase in a critical ill patient according to any of claims 22-24, wherein said shock is selected from the group comprising cardiogenic shock or septic shock.

26. A DPP3 inhibitor for use in the prevention of a DPP3 increase in a critical ill patient, wherein said patient has a level of DPP3 above a threshold, wherein said threshold is between 40 ng/ml and 22 ng/ml and wherein said DPP3 inhibitor is an anti-DPP3-antibody and/or and anti-DPP3-antibody fragment and/or anti-DPP3 scaffold.
